(19) United States
(12) Patent Application Publication
(10) Pub. No.: US 2020/0208223 A1
(43) Pub. Date: Jul. 2, 2020

(54) IDENTIFICATION OF EPIGENETIC SIGNATURES INDICATING BREAST CANCER

(71) Applicants: Christiana Care Health Services, Inc., Newark, DE (US); Genome Profiling, LLC, Newark, DE (US)

(72) Inventors: Jennifer Sims-Mourtada, Wilmington, DE (US); Adam Marsh, Lewes, DE (US)

(73) Assignees: Christiana Care Health Services, Inc., Newark, DE (US); Genome Profiling, LLC, Newark, DE (US)

(21) Appl. No.: 16/643,043

(22) PCT Filed: Sep. 12, 2018
(86) PCT No.: PCT/US2018/050651

§ 371 Date: Feb. 28, 2020

Related U.S. Application Data
(60) Provisional application No. 62/558,124, filed on Sep. 13, 2017.

(51) Int. Cl. C12Q 1/6886

(52) U.S. Cl. CPC C12Q 1/6886; C12Q 2600/112; C12Q 2600/154

(57) ABSTRACT

In various aspects and embodiments, the invention provides a method of determining breast cancer status of a subject, the method comprising determining a methylation state for each of a plurality of cytosine-guanine dinucleotide (CpG) sites in a sample obtained from the subject, calculating a cancer presence differential methylation level and an invasiveness differential methylation level based on the methylation states of the plurality of CpG sites, and comparing the cancer presence differential methylation level and the invasiveness differential methylation level to a predetermined cancer status reference level and a predetermined invasiveness reference level, wherein when the cancer presence differential methylation level deviates from the predetermined cancer status reference level, the presence of breast cancer is indicated in the subject, and when the invasiveness differential methylation level deviates from the predetermined invasiveness reference level, the presence of invasive breast cancer is indicated in the subject.

### CROSS REFERENCE TO RELATED APPLICATIONS

The present application claims priority under 35 U.S.C. § 119(e) to U.S. Provisional Patent Application No. 62/558,124, filed Sep. 13, 2017, which is incorporated herein by reference in its entirety.

### BACKGROUND OF THE INVENTION

Breast cancer is the most commonly diagnosed cancer in women worldwide. Although the intent of increased mammographic screening is early detection of invasive cancer, an unexpected consequence has been a substantial increase in the number of women diagnosed with ductal carcinoma in situ (DCIS). DCIS is considered a benign lesion that consists of non-invasive neoplastic cells. Although DCIS has been considered a precursor lesion to invasive breast cancers, only a small percentage of DCIS cases progress to invasive cancers. The most common treatment for DCIS is breast-conserving surgery followed by radiation. Recently, there has been considerable debate over the treatment of DCIS, with many claiming that current treatment regimens result in overtreatment of a mostly indolent disease.

While some groups recommend frequent follow up and monitoring of women with low-grade DCIS instead of invasive treatment, this approach would necessitate either frequent biopsy, an unattractive option for most women, or additional mammography. Additionally, for some women, mammography may be hard to interpret or may provide a false negative. In particular, dense stromal and epithelial tissue within the breast may cause high background and women with high breast density are more at risk for a false-negative mammogram. Likewise, certain forms of breast cancer such as triple-negative breast cancer (TNBC) are less likely to be detected by mammographic screening. Although TNBC may have larger size at diagnosis compared with other breast cancer subtypes, up to 18% of TNBCs remain unidentified on initial screening. This is due to the fact TNBC lacks the typical suspicious mammographic features of breast cancer, such as irregular mass shape, spiculated margins, associated ductal carcinoma in situ and suspicious calcifications. Thus mammography alone may be a suboptimal test for diagnosis of TNBC, especially for those at high risk.

### SUMMARY OF THE INVENTION

In one aspect, the invention provides a method of determining breast cancer status of a subject, the method comprising:

determining a methylation state for each of a plurality of cytosine-guanine dinucleotide (CpG) sites in a sample obtained from the subject,

calculating a cancer presence differential methylation level and an invasiveness differential methylation level based on the methylation states of the plurality of CpG sites, and

comparing the cancer presence differential methylation level and the invasiveness differential methylation level to a predetermined cancer status reference level and a predetermined invasiveness reference level,

wherein when the cancer presence differential methylation level deviates from the predetermined cancer status reference level, the presence of breast cancer is indicated in the subject, and

when the invasiveness differential methylation level deviates from the predetermined invasiveness reference level, the presence of invasive breast cancer is indicated in the subject.

In another aspect, the invention provides a method of detecting breast cancer in a subject, the method comprising:

determining a methylation state for each of a plurality of cytosine-guanine dinucleotide (CpG) sites in a sample obtained from the subject,

calculating a cancer status differential methylation level based on the methylation states of the plurality of CpG sites, and

comparing the cancer status reference differential methylation level to a predetermined reference level,

wherein when the cancer status differential methylation level deviates from the predetermined reference level, the presence of breast cancer is indicated in the subject.

In another aspect, the invention provides a method of determining if breast cancer in a subject is invasive, or non-invasive, the method comprising:

determining a methylation state for each of a plurality of cytosine-guanine dinucleotide (CpG) sites in a sample obtained from the subject,

calculating an invasiveness differential methylation level based on the methylation states of the plurality of CpG sites, and

comparing the invasiveness differential methylation level to a predetermined reference level,

wherein when the differential methylation level deviates from the predetermined reference level, the breast cancer in the subject is invasive.

In various embodiments, the plurality of CpG sites comprises at least one selected from the CpG sites listed in Table 3 or Table 15.

In various embodiments, the plurality of CpG sites comprises at least five selected from the CpG sites listed in Table 21.

In various embodiments, the plurality of CpG sites comprises at least ten selected from the CpG sites listed in Table 3 or Table 15.

In various embodiments, the plurality of CpG sites comprises at least ten selected from the CpG sites listed in Table 21.

In various embodiments, the plurality of CpG sites comprises at least m % selected from the top n most predictive CpG sites listed in Table 3 and/or Table 15, wherein:

m is selected from the group consisting of: 50, 60, 70, 80, 90, 95, and 99; and

n is selected from the group consisting of 25, 50, 100, 500 and 1,000.

In various embodiments, the plurality of CpG sites comprises at least m % selected from the top n most predictive CpG sites listed in Table 21, wherein:

m is selected from the group consisting of: 50, 60, 70, 80, 90, 95, and 99; and

n is selected from the group consisting of 25, 50, 100, 500 and 1,000.

In various embodiments, the method further comprises providing treatment for breast cancer to the subject when breast cancer is indicated.

In various embodiments, the treatment for breast cancer comprises the administration of medication, radiation or surgery.

In various embodiments, calculating a differential methylation level comprises adding in a linear weighted summation values based on the methylation states of the plurality of CpG sites.

In various embodiments, the sample is a blood sample.

In various embodiments, the sample is tumor tissue.

In various embodiments, the subject has or is suspected to have ductal cell in situ carcinoma.

In various embodiments, the subject has or is suspected to have triple-negative breast cancer.

In various embodiments, the subject has or is suspected to have hormone receptor positive (ER+PR+) breast cancer.

In various embodiments, the subject has or is suspected to have HER2+ breast cancer.

In various embodiments, the subject is being monitored for the local or systemic recurrence of breast cancer.

In various embodiments, the plurality of CpG sites includes at least one selected from table 27.

### BRIEF DESCRIPTION OF THE DRAWINGS

For a fuller understanding of the nature and desired objects of the present invention, reference is made to the following detailed description taken in conjunction with the accompanying figures.

FIG. 1A depicts % methylation frequency profiles in individual samples from the control, invasive and DCIS subject groups.

FIG. 1B depicts a comparison of differential methylation load by gene functional class and domain structure for control and DCIS subject groups.

FIG. 1C depicts a non-metric multidimensional scaling analysis to identify discriminating 5mC patterns among all three subject groups.

FIG. 1D depicts the distribution of the strength vectors of CpG sites contributing to the group separation in the NMDS plot depicted in FIG. 1C.

FIGS. 2A-C depicts CpG methylation as a heatmap for the top 1,000 statistically significant sites based on a Likelihood-Ratio-Test (LRT) of a one-way ANOVA contrast for the three-way analysis. Table 8 contains the counts of the top 40 statistically significant sites.

FIG. 2D depicts a hierarchical clustering of methylation patterns among top CpG sites to identify those sites or domains with correlated shifts in methylation.

FIGS. 3A-C depict CpG methylation distribution from LRT in FIG. 2A-C, respectively.

FIGS. 3D-F depict CpG fold-change vs p-val in volcano plots from LRT in FIG. 2A-C, respectively.

FIG. 4 depicts methylation load across the genome.

FIG. 5A depicts % methylation frequency profiles in individual samples from the control and DCIS subject groups.

FIG. 5B depicts a comparison of differential methylation load by gene functional class and domain structure for control and DCIS subject groups.

FIG. 5C depicts a non-metric multidimensional scaling analysis to identify discriminating 5mC patterns among control and DCIS subject groups.

FIG. 5D depicts the distribution of the strength vectors of CpG sites contributing to the group separation in the NMDS plot depicted in FIG. 5C.

FIG. 6A depicts CpG methylation as a heatmap for the top 1,000 statistically significant sites based on a Likelihood-Ratio-Test of a one-way ANOVA contrast for the control and DCIS subject groups. Table 14 contains the counts of the top 40 statistically significant sites.

FIG. 6B depicts a hierarchical clustering of methylation patterns among top CpG sites to identify those sites or domains with correlated shifts in methylation.

FIG. 7A depicts CpG response distribution in smear plots from LRT in FIG. 6A.

FIG. 7B depicts CpG response distribution in volcano plots from LRT in FIG. 6A. depicts a heatmap clustering of the top 1,000 CpG sites ranked by p-value.

FIG. 7C depicts methylation load across the genome.

FIG. 8A depicts % methylation frequency profiles in individual samples from the control and invasive breast cancer subject groups.

FIG. 8B depicts a comparison of differential methylation load by gene functional class and domain structure for control and invasive breast cancer subject groups.

FIG. 8C depicts non-metric multidimensional scaling analysis to identify discriminating 5mC patterns among control and invasive breast cancer subject groups.

FIG. 8D depicts the distribution of the strength vectors of CpG sites contributing to the group separation in the NMDS plot depicted in FIG. 8C.

FIG. 9A depicts CpG methylation as a heatmap for the top 1,000 statistically significant sites based on a Likelihood-Ratio-Test of a one way ANOVA contrast for the control and invasive breast cancer subject groups. Table 20 contains the counts of the top 40 statistically significant sites.

FIG. 9B depicts a hierarchical clustering of methylation patterns among top CpG sites to identify those sites or domains with correlated shifts in methylation.

FIG. 10A depicts CpG response distribution in smear plots from LRT in FIG. 9A.

FIG. 10B depicts CpG response distribution in volcano plots from LRT in FIG. 9A.

FIG. 10C depicts methylation load across the genome.

FIG. 11A depicts % methylation frequency profiles in individual samples for the DCIS and invasive breast cancer subject groups.

FIG. 11B depicts a comparison of differential methylation load by gene functional class and domain structure for DCIS and invasive breast cancer subject groups.

FIG. 11C depicts a non-metric multidimensional scaling analysis to identify discriminating 5mC patterns between DCIS and invasive breast cancer subject groups.

FIG. 11D depicts distribution of the strength vectors of CpG sites contributing to the group separation in the NMDS plot in FIG. 11C.

FIG. 12A depicts CpG methylation as a heatmap for the top 1,000 statistically significant sites based on a Likelihood-Ratio-Test of a one way ANOVA contrast for the DCIS and invasive breast cancer subject groups. Table 26 contains the counts of the top 40 statistically significant CpG sites.

FIG. 12B depicts a hierarchical clustering of methylation patterns among top CpG sites to identify those sites or domains with correlated shifts in methylation.

FIG. 13A depicts CpG response distribution in smear plots from LRT in FIG. 12A.

FIG. 13B depicts CpG response distribution in volcano plots from LRT in FIG. 12A.

FIG. 13C depicts methylation load across the genome.

FIG. 14 is an NMDS plot for the replicate cohort used to show the separation on blood DNA methylation profiles between healthy women, women with advanced breast cancer (“invasive”), and women with DCIS lesions.

FIG. 15 is a heat map of top 1,000 statistically significant CpG sites when comparing CON and INV patient groups. These CpG sites and methylation load scores for CON and INV patients were used in the classification platform.

FIG. 16 depicts pie charts presenting the classification calls for the 10 blind samples. The platform uses a voting/polling approach and so the pie charts present the proportion of votes that each sample received for each phenotype class (CON or INV). Decisions were made based on simple majority counts.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “breast cancer” refers to pre-malignant or malignant tumors that start in the epithelial cells of the breast. Breast cancer can be further defined into pathological subtypes based on expression of the estrogen, progesterone, and HER2 receptors. Additionally, breast cancer can be defined based on molecular subtype as defined, for example, by Peru et al, Nature. 2000 Aug. 17; 406(6797):747-5. Molecular subtypes include luminal A, B, and C, HER 2, basal-like, and normal type.

As used in the specification and claims, the terms “comprises,” “comprising,” “containing,” “having,” and the like can have the meaning ascribed to them in U.S. patent law and can mean “includes,” “including,” and the like.

As used herein, “cytosine-guanine dinucleotide site” or “CpG site” means a cytosine nucleotide followed by a guanine nucleotide in the genome of an organism. CpG sites can be designated with the number of the chromosome of the organism on which they are located and a number designating the position. The flanking sequences can be used to generate the position number. For example, “12.108079458” or “chr12 108079458” refer to a CpG site on chromosome 12 at position 108079458. The position number refers to the nucleotide index starting from 1 on the coding or plus (+) strand of the DNA molecule and specifically references the position of the 5′ cytosine in a CpG dinucleotide pair. In addition, this CpG location has a complementary sequence pair on the non-coding minus (−) strand and the position number also refers to that complementary strand cytosine which is located plus one nucleotide from the indicated coding strand position. Thus, for CpG 12.108079458, the methylation score values indexed to this specific site cover the cytosine on the coding strand of chromosome 12 at position number 108079458 and the cytosine on the non-coding strand of chromosome 12 at position number 108079459.

As used herein, the term “carcinoma in situ” refers to a neoplastic lesion characterized by increased epithelial proliferation, subtle to marked cellular atypia and an inherent but not necessarily obligate tendency for progression to invasive breast cancer as defined by Lakhani S, Ellis I, Schnitt S, et al. 4th. Lyon: IARC Press; 2012. WHO Classification of Tumours of the Breast. Lobular carcinoma in situ (LCIS) refers to a lesion originating from the terminal ductal lobular units, whereas, ductal carcinoma in situ (DCIS) refers to an intraductoral lesion. DCIS is also known as intraductal carcinoma, ductal intraepithelial neoplasia and includes the following subtypes cribiform, solid, comedo, papillary, and micropapilary. DCIS can also be characterized as low, moderate and high grade.

As used herein, the term “invasive breast cancer” means a malignant epithelial tumor of the breast, characterized by invasion of adjacent tissues and a marked tendency to metastasize to distant sites as defined by Lakhani S, Ellis I, Schnitt S, et al. 4th. Lyon: IARC Press; 2012. WHO Classification of Tumours of the Breast. As used herein, the term encompasses both cancer that presently exhibits these qualities and cancer that will develop them over the course of disease progression. Invasive breast cancer includes the following subtypes: invasive carcinoma of no special subtype, invasive carcinoma of mixed type, invasive ductal carcinoma, invasive lobular carcinoma, infiltrating carcinoma of the breast, tubular carcinoma, invasive cribriform carcinoma, carcinoma with medullary features, mucinous carcinoma, invasive papillary carcinoma, inflammatory breast cancer, Paget disease of the breast, metaplastic breast cancer, carcinomas with aprocrine differentiation, adenoid cystic carcinoma, microinvasive carcinoma, and carcinoma with neuroendrocrine features.

As used herein, “hormone receptor positive breast cancer” and “HER2+” mean cancers which have positive expression of estrogen or progesterone receptors in greater than 1-9% percent of cells. Hormone receptor positive tumors may or may not have overexpression of the HER2 receptor noted either by immunohistochemical or genetic evaluation.

As used herein, “methylation” or “methylated” as applied to CpG sites refers to the addition of a methyl group to cytosine, forming either 5′-methyl-cytosine or 5′-hydroxymethyl-cystosine.

As used herein, the term “percent methylation” or “% MET” refers to the frequency with which a particular set of CpG sites are methylated. Here, CpG methylation is expressed as a percentage of methylated copies found in the DNA sample for each individual CpG site relative to the total number of copies found for each site.

A “reference level” with respect to some measurement used in diagnosis is indicative of the presence or absence of a particular phenotype or characteristic. When the level of the measurement in a subject deviates from the reference level it is indicative of the presence of, or relatively heightened level of, a particular phenotype or characteristic.

As used herein, the term “triple negative breast cancer” means breast cancer in which less than 1-9% of cells express the estrogen or progesterone receptors and there is no alteration of the HER2 receptor noted either by immunohistochemical or genetic evaluation. The majority of triple negative breast cancer have gene expression profiles that are representative of the basal subtype of breast cancer.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

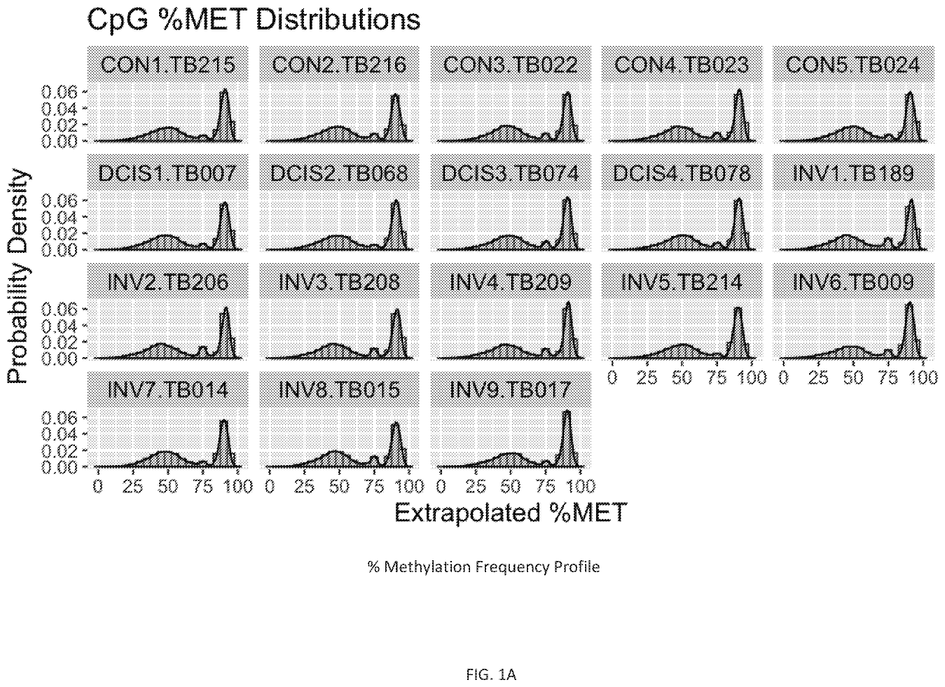

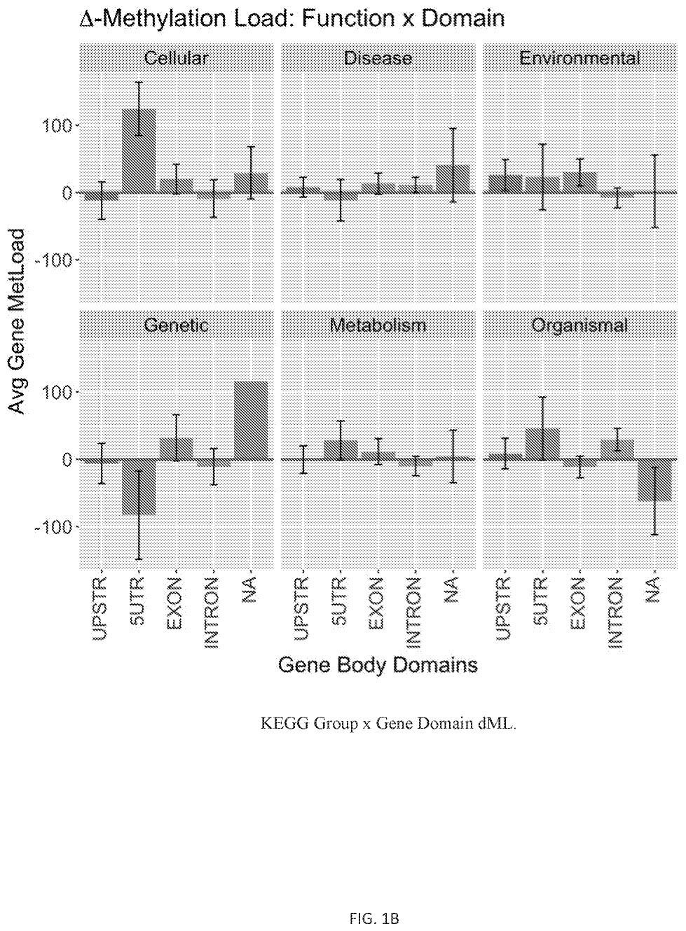

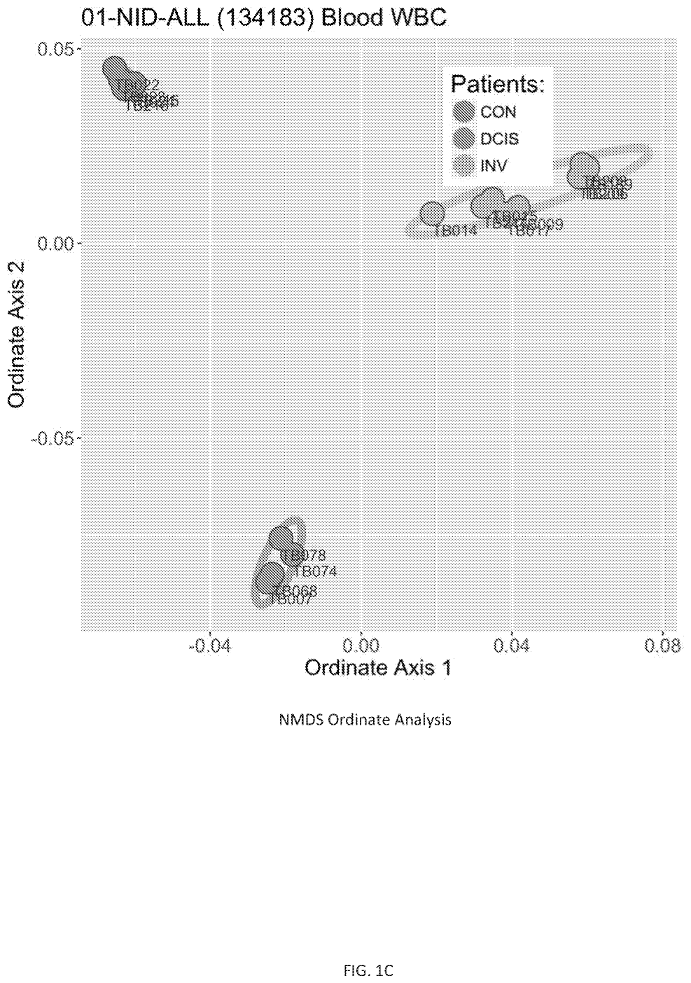

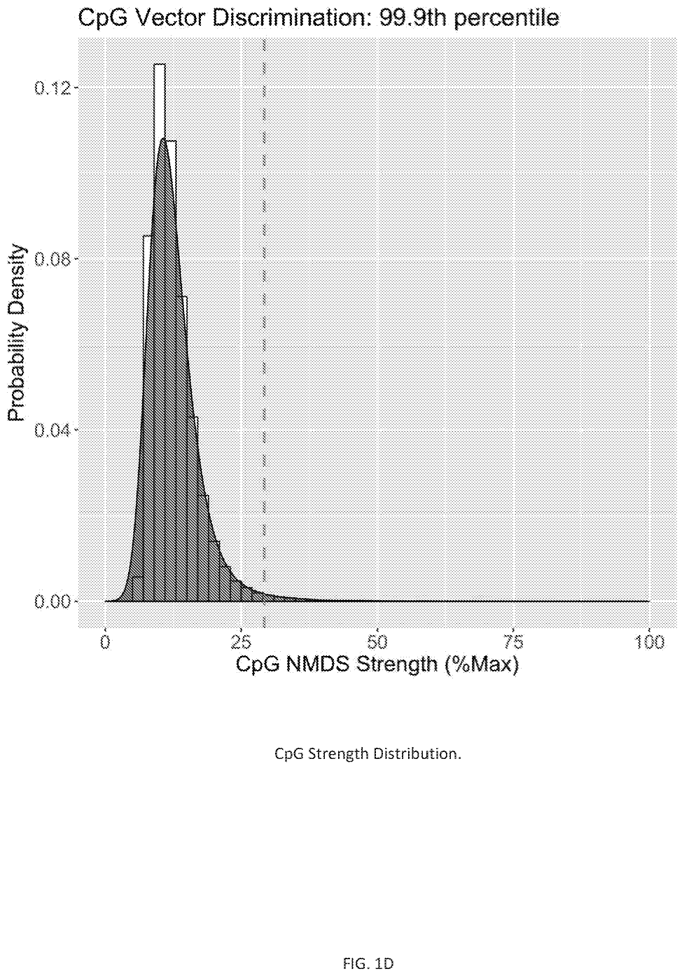

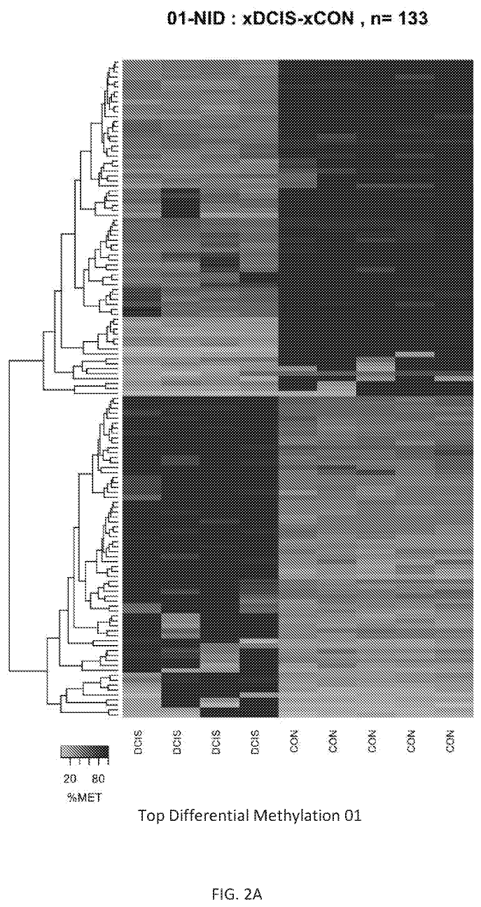

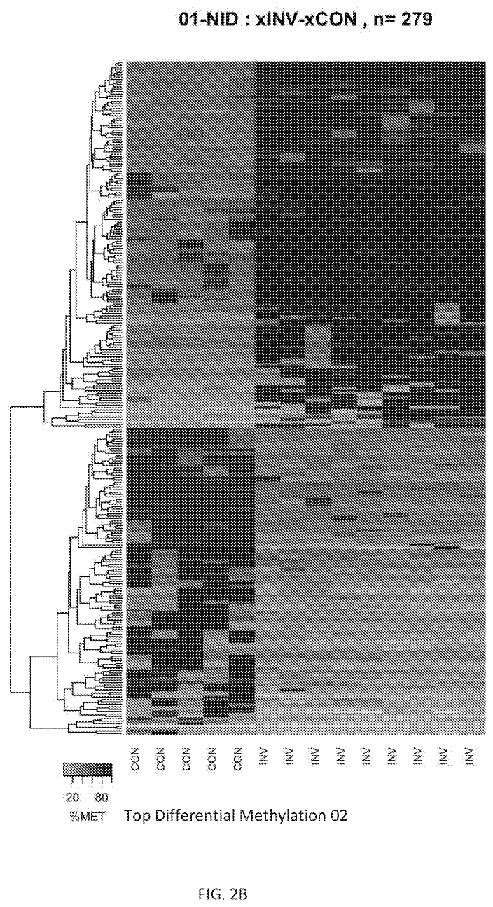

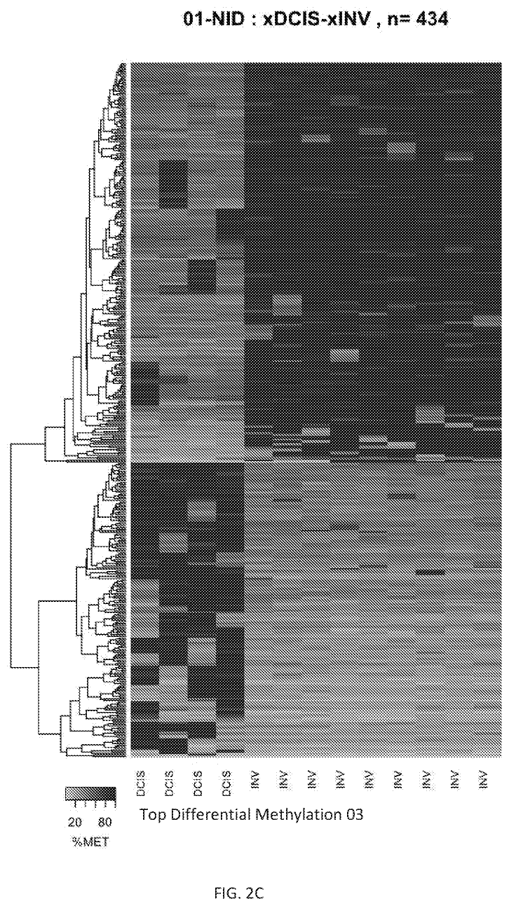

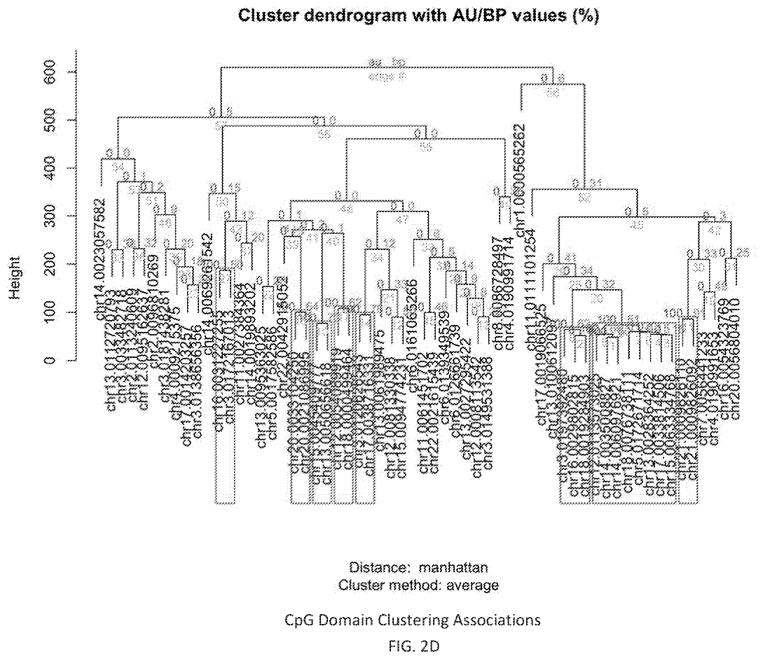

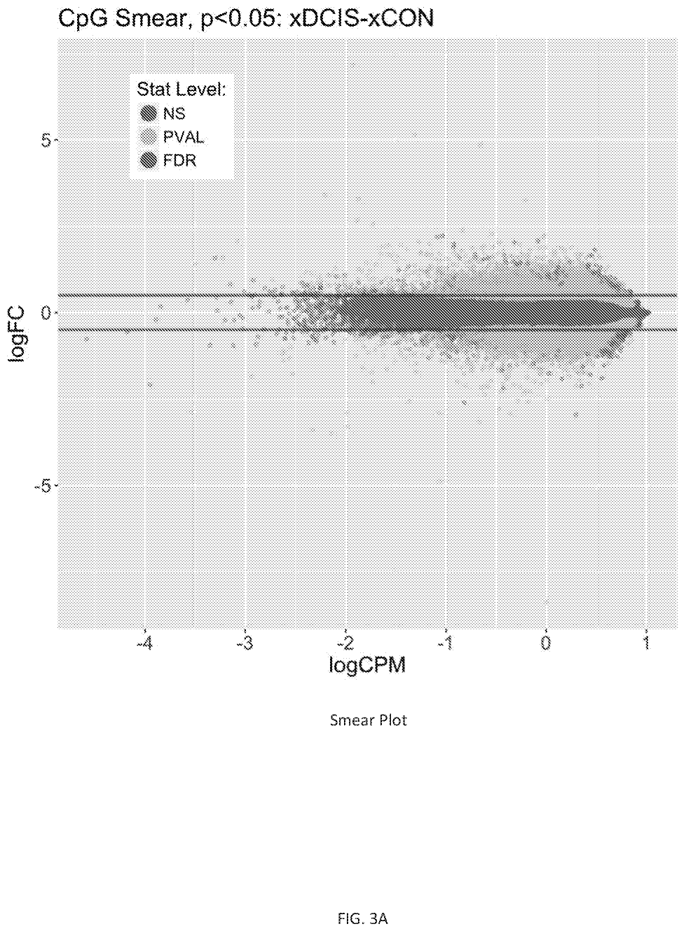

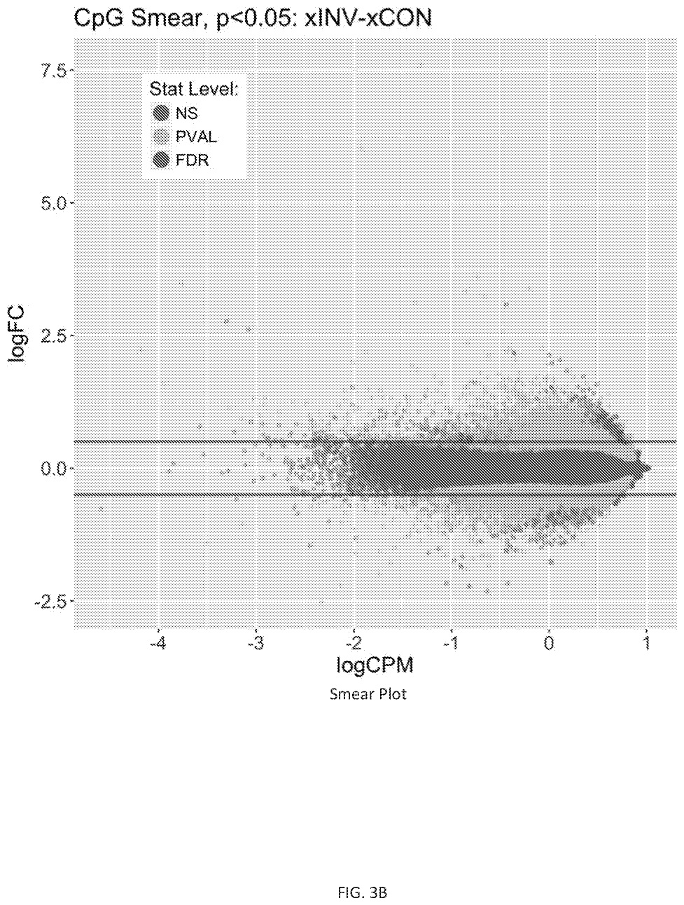

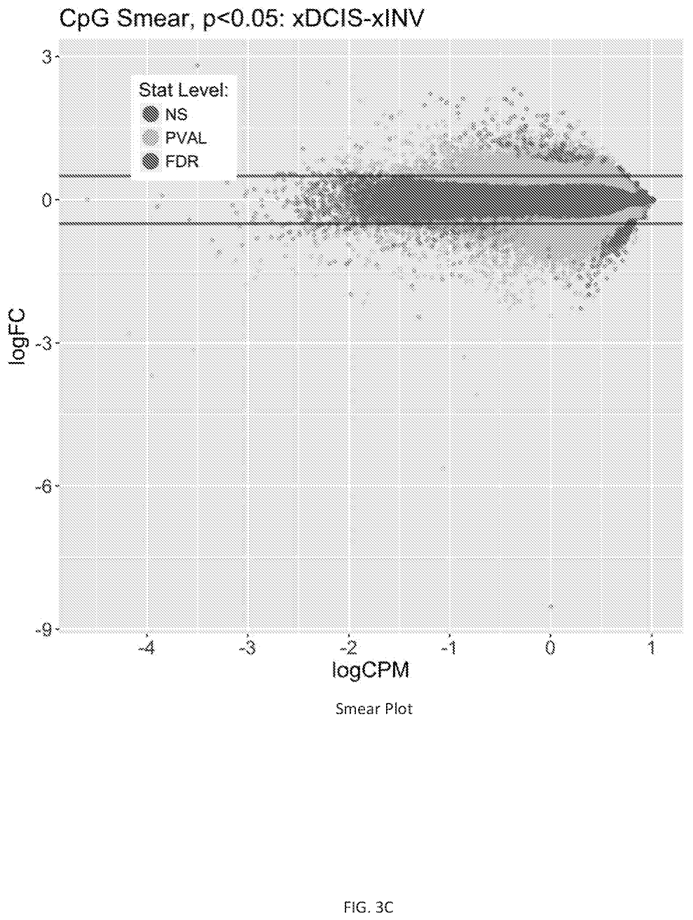

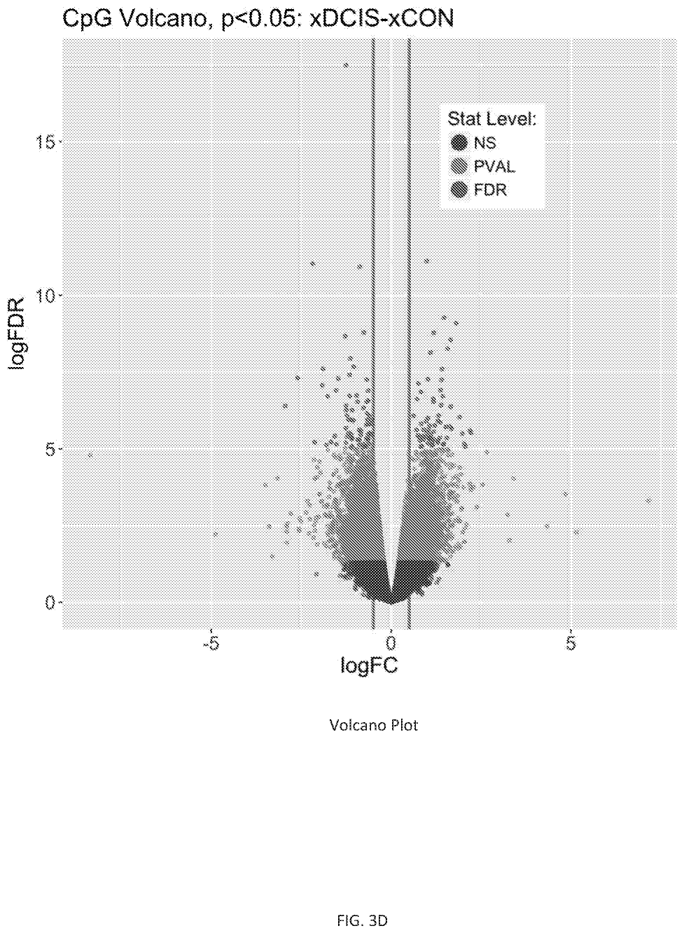

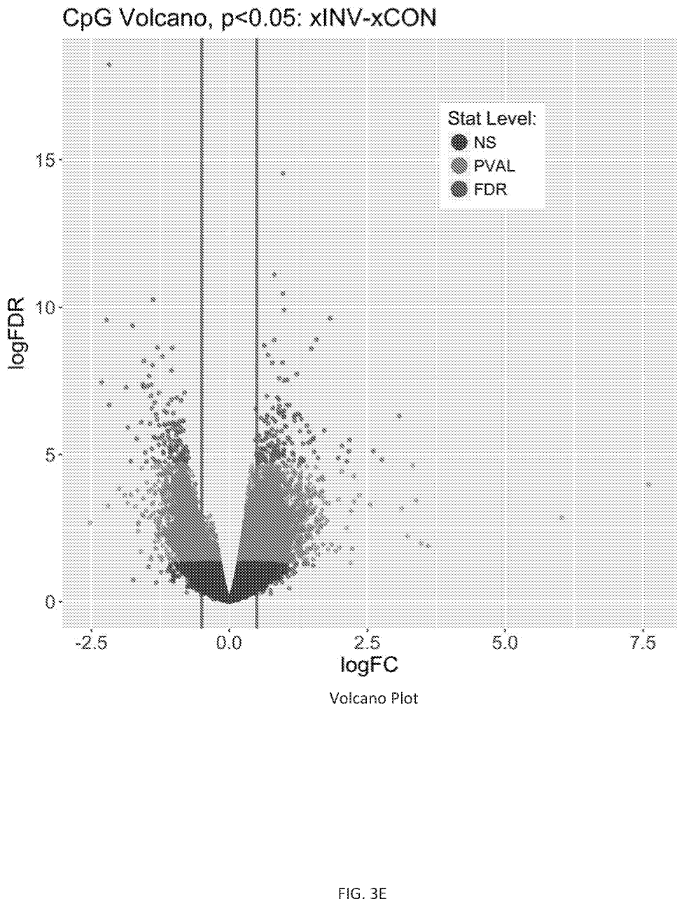

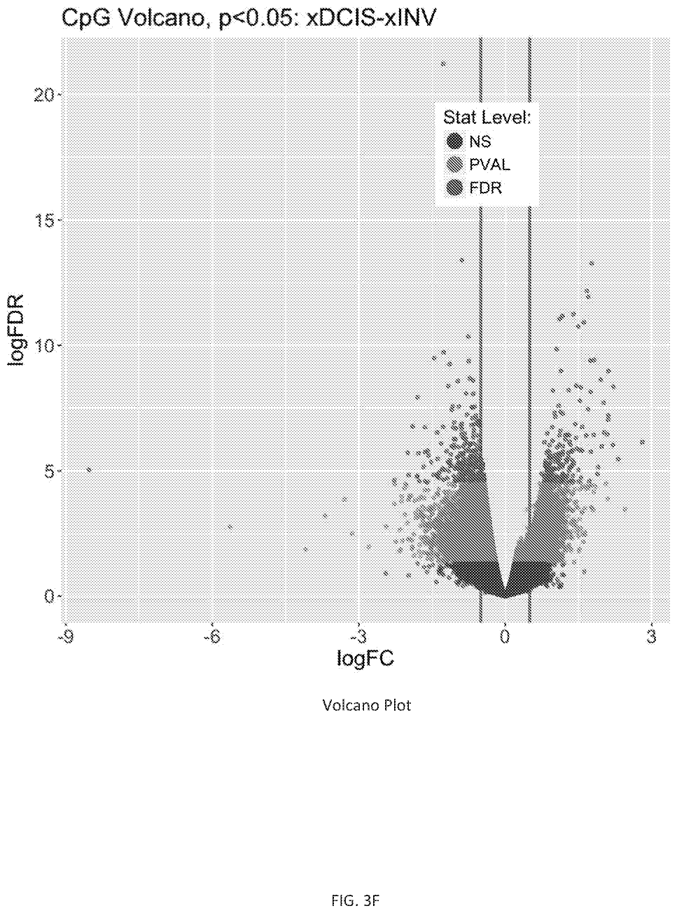

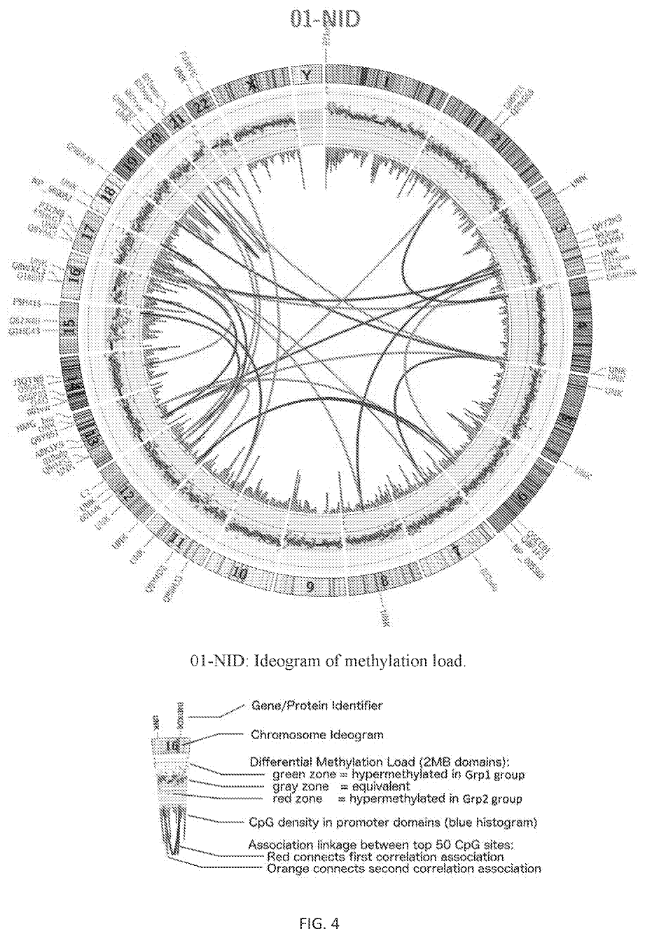

In one aspect, the invention provides a method of determining the breast cancer status of a subject by determining a methylation state for each of a plurality of cytosine-guanine dinucleotide (CpG) sites in a sample obtained from the subject, calculating a cancer status differential methylation level and an invasiveness differential methylation level based on the methylation states of the plurality of CpG sites, and comparing the cancer presence differential methylation level and the invasiveness differential methylation level to a predetermined cancer status reference level and a predetermined invasiveness reference level, wherein when the cancer status differential methylation level deviates from the predetermined cancer status reference level, the presence of breast cancer is indicated in the subject, and when the invasiveness differential methylation level deviates from the predetermined invasiveness reference level, the presence of invasive breast cancer is indicated in the subject. This aspect of the invention is based in part on the three-way analysis illustrated in FIGS. 1-4 and discussed further in the example below.

Aspects of the invention may be applied to determine if a patient is free from breast cancer, has DCIS or has invasive breast cancer. This may be achieved by determining if the patient's epigenetic profile, based on a plurality of CpG sites and expressed as a cancer presence differential methylation level and an invasiveness differential methylation level, deviates from the epigenetic profile of patients without breast cancer or with DCIS, expressed as a cancer presence reference level and invasiveness reference level. When the patient's cancer presence differential methylation level deviates from the cancer presence reference level, the presence of cancer (invasive or not) is indicated. When the patient's invasiveness differential methylation level, deviates from the invasiveness reference level, the presence of invasive breast cancer is indicated. In various embodiments, the subjects may have invasive breast cancer associated with carcinoma in situ. A person of skill in the art will appreciate that the methods of the invention may be used to detect this condition by calculating a carcinoma in situ differential methylation level based on a plurality of the CpG sites disclosed herein using the below described methods.

In another aspect, the invention provides a method of detecting breast cancer in a subject by determining a methylation state for each of a plurality of cytosine-guanine dinucleotide (CpG) sites in a sample obtained from the subject, calculating a cancer status differential methylation level based on the methylation states of the plurality of CpG sites, and comparing the cancer status differential methylation level to a predetermined cancer status reference level, wherein when the differential methylation level deviates from the predetermined cancer status reference level, the presence of breast cancer is indicated in the subject. The methods of the invention need not be applied to determine if the cancer is invasive. The method may also be applied to detect cancer by determining if the patient's epigenetic profile, based on a plurality of CpG sites and expressed as a cancer status differential methylation level, deviates from the epigenetic profile of patients without breast cancer, expressed as a cancer status reference level. When the patient's cancer status differential methylation level deviates from the cancer status reference level, the presence of breast cancer in the patient is indicated.

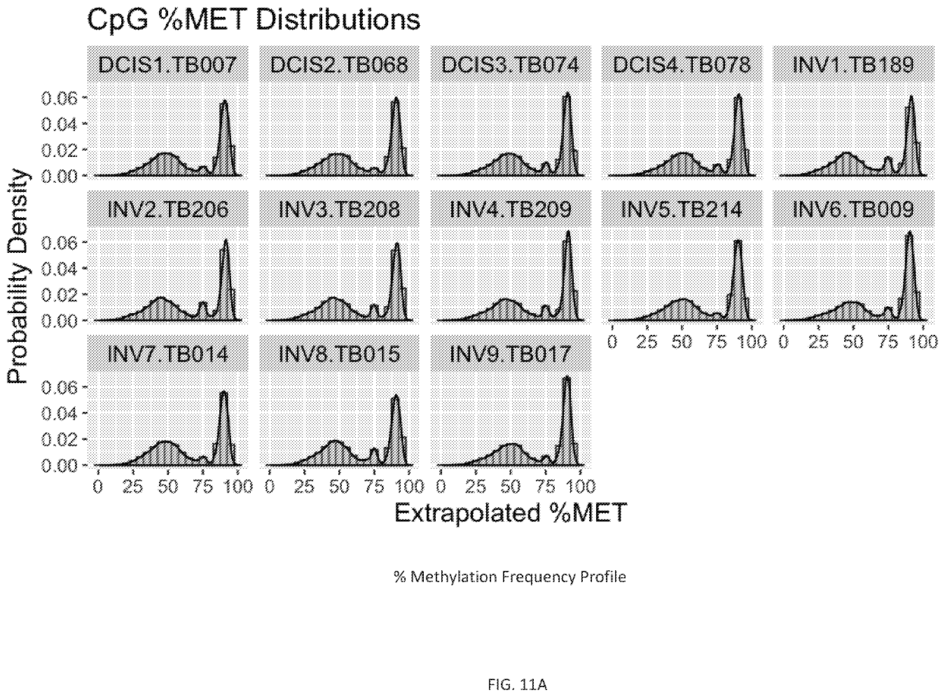

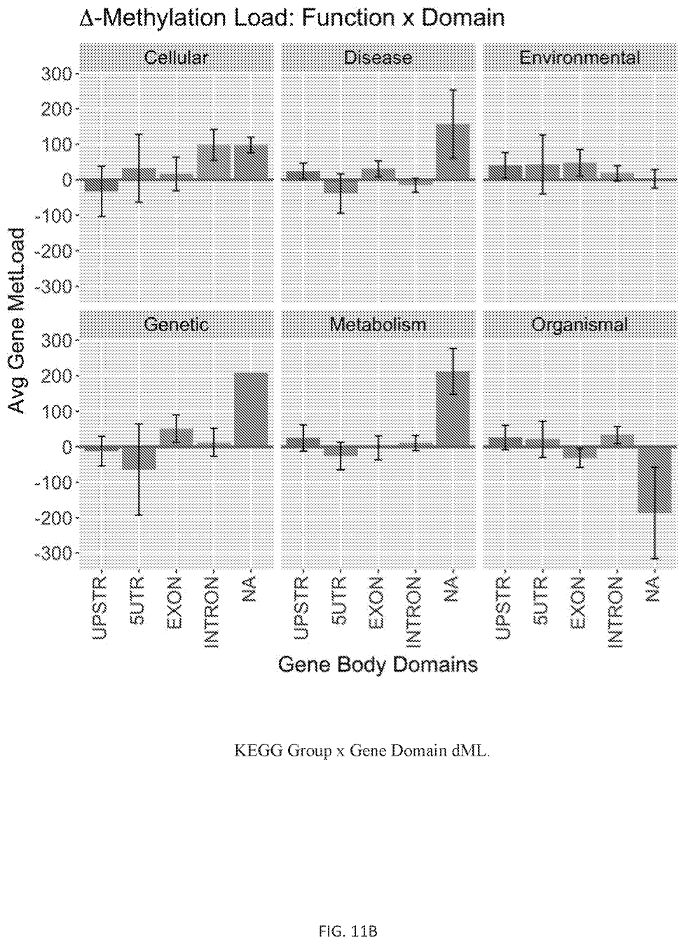

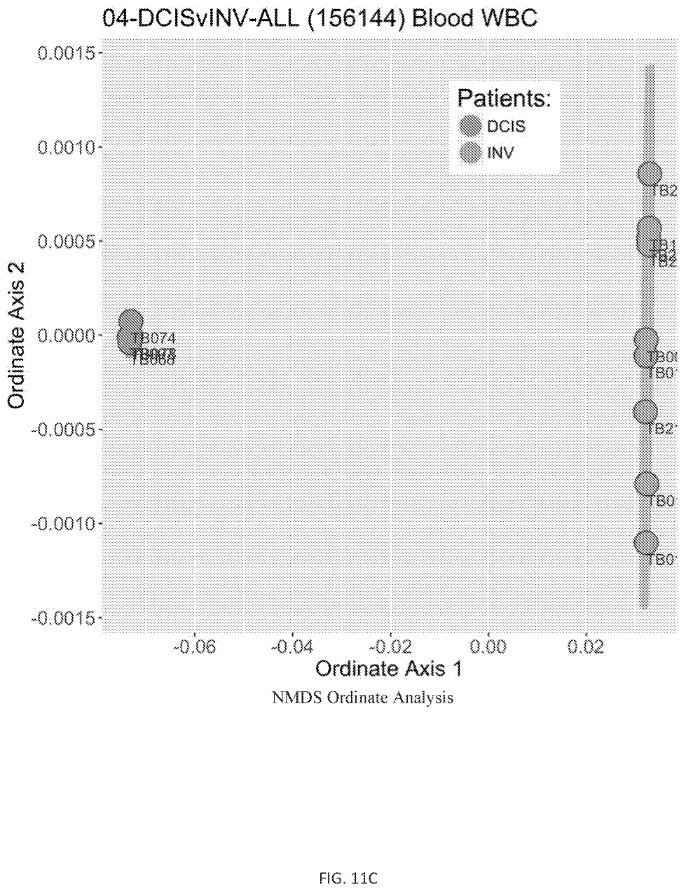

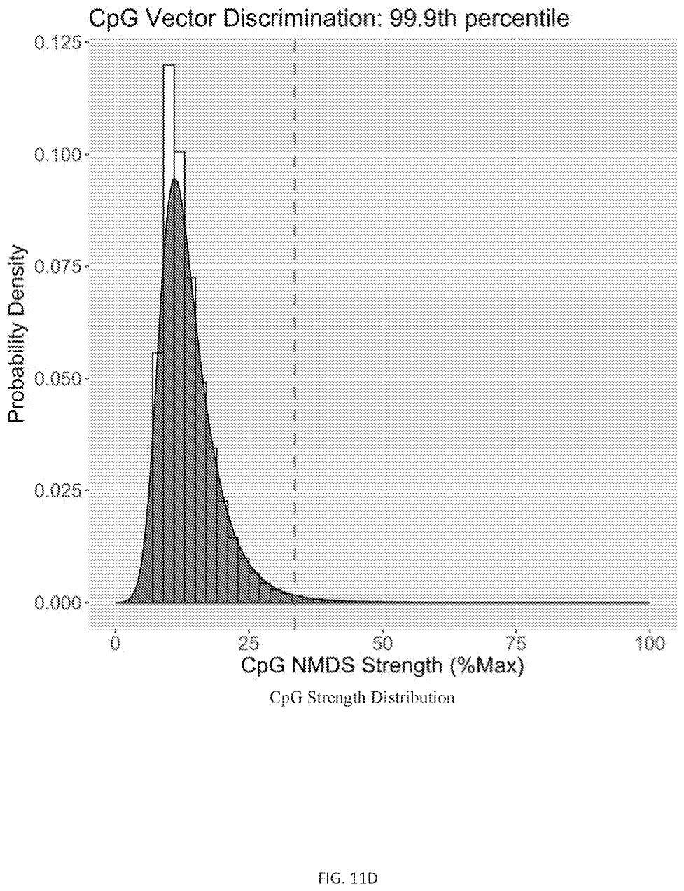

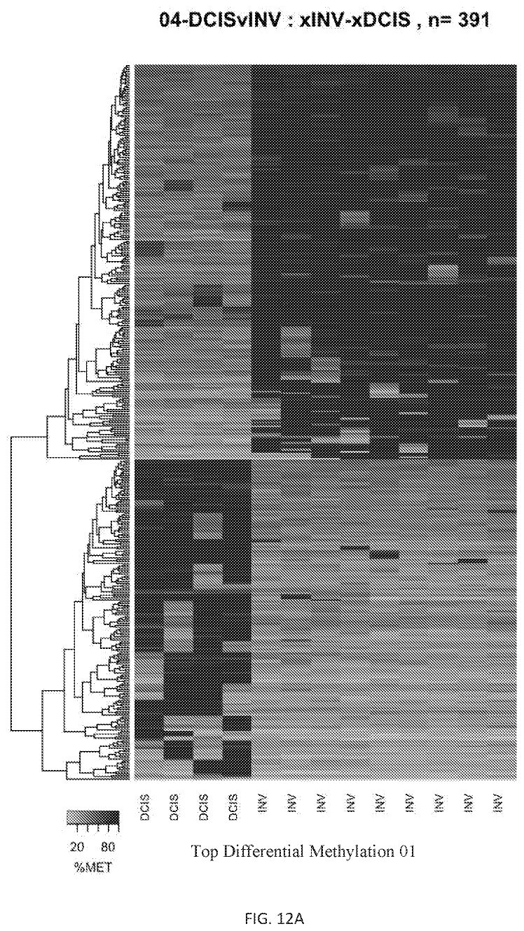

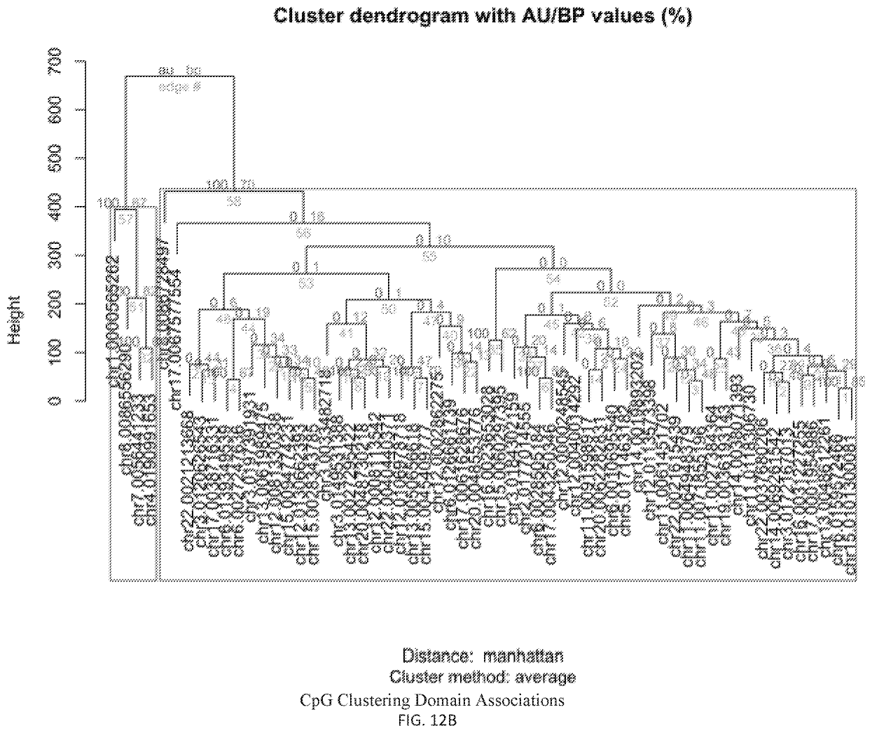

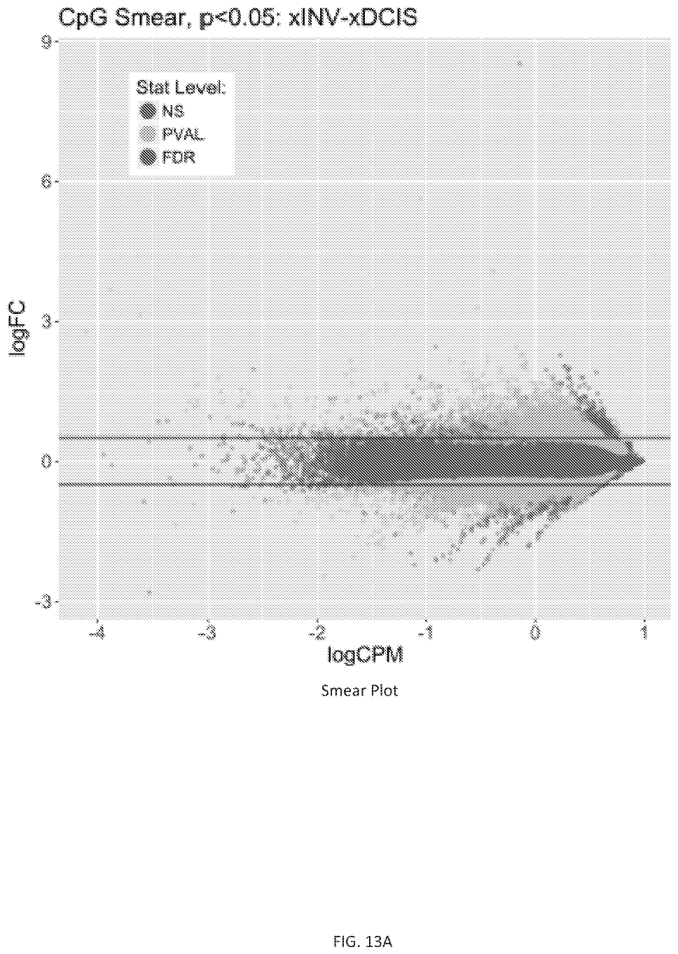

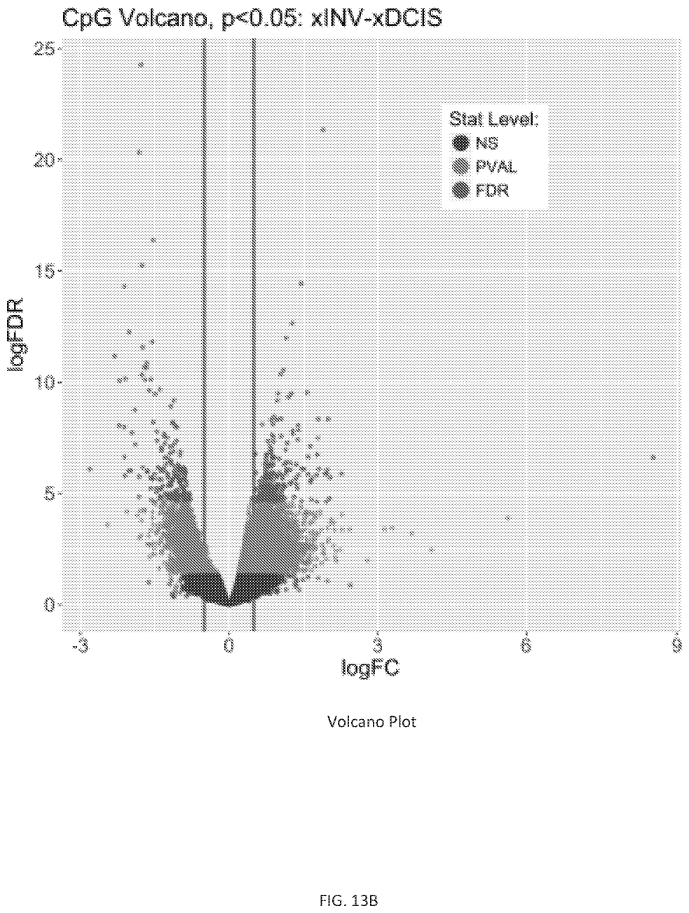

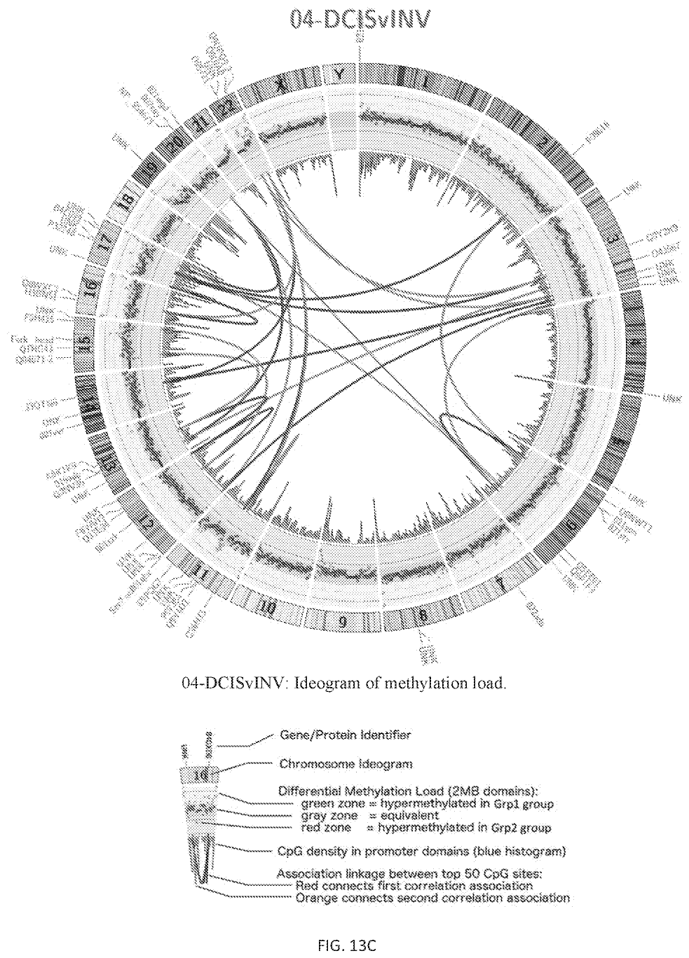

In another aspect, the invention provides a method of determining if breast cancer in a subject is invasive or non-invasive by determining a methylation state for each of a plurality of CpG sites in a sample obtained from the subject, calculating an invasiveness differential methylation level based on the methylation states of the plurality of CpG sites, and comparing the invasiveness differential methylation level to a predetermined reference level, wherein when the differential methylation level deviates from the predetermined reference level, the presence of breast cancer is indicated in the subject. This aspect of the invention is related to the DCIS vs. invasive analysis illustrated in FIGS. 11-13 and discussed further in the example below.

The methods of the invention may be applied to patients who have previously been diagnosed or are otherwise believed to have breast cancer, in order to determine whether or not the cancer is invasive. When the patient's invasiveness differential methylation level deviates from the invasiveness reference level, the presence of invasive breast cancer is indicated.

In another aspect, the invention provides a method of detecting local or systemic recurrence of breast cancer by determining a methylation state for each of a plurality of cytosine-guanine dinucleotide (CpG) sites in a sample obtained from the subject, calculating a cancer status differential methylation level and an invasiveness differential methylation level based on the methylation states of the plurality of CpG sites, and comparing the cancer presence differential methylation level and the invasiveness differential methylation level to a predetermined cancer status reference level and a predetermined invasiveness reference level, wherein when the cancer status differential methylation level deviates from the predetermined cancer status reference level, the recurrence of breast cancer is indicated in the subject, and when the invasiveness differential methylation level deviates from the predetermined invasiveness reference level, the recurrence of invasive breast cancer is indicated in the subject.

In various embodiments, the patient has or is suspected to have triple negative breast cancer. In various embodiments, the patient has or is suspected to have hormone receptor positive (ER+PR+, or ER+PR− or ER− PR+) breast cancer. In various embodiments, the patient has or is suspected to have HER2+ breast cancer.

The methylation of state of the plurality of CpG sites may be determined by any means known in the art. In various embodiments, the methylation state of the plurality of CpG sites may be determined by methyl-sensitive restriction enzyme digestion followed by Next-Gen Sequencing (NGS) on an appropriate instrument, or they may be determined by targeted qPCR assays to quantify cut and uncut CpG sites following methyl-sensitive restriction enzyme digestion, or they may be determined by bisulfite oxidation treatment with DNA sequencing (either direct or via NGS), or they may be determined by hybridization of labelled oligonucleotide probes (called “hybridization arrays”) to measure methylation following methyl-sensitive restriction enzyme digestion, or they may be determined by hybridization of anti-5′-methyl-cytosine antibodies to measure methylation after hybridization capture on a targeted gene panel.

Without wishing to be limited by theory, in various embodiments, the method relies on the concept of differential methylation level (ΔML)—site-specific differences in CpG methylation summed across a gene or genome domain, structure or element—in order to characterize functional shifts in methylation patterns. This method is illustrated in the example below and in Equation (1). In various embodiments, calculating a differential methylation level comprises adding in a linear weighted summation values based on the methylation states of the plurality of CpG sites.

As shown in FIGS. 1D, 5D, 8D, and 11D, the methylation state of the majority of CpG sites in the genome are not significant predictors of breast cancer in a patient. However, determining the methylation state of a certain subset of CpG sites and applying the algorithm in the example below or a derivative classification algorithm, one may be used to determine the breast cancer status of a patient. The plurality of CpG sites in the present aspect of the invention refers to sites selected from this predictive subset of sites. A list of predictive sites and their predictive power as measured by p-value and ordinate discrimination is presented in the Appendix.

In various embodiments, the plurality of CpG sites includes a plurality of “up sites” and a plurality of “down sites”. Up sites are CpG sites where methylation at the site indicates an increased methylation load in patients in the first group in the comparison relative to patients in the second group in the comparison. Down sites are CpG sites where methylation at the site indicates a decreased methylation load in the first group in the comparison relative to patients in the second group in the comparison.

Various embodiments of the invention are directed to methods of examining sets of up sites and down sites and determining a differential methylation level based on their methylation state. Due to the predictive value of the plurality of sites, the scores will differ when examining patients with and without breast cancer and where the breast cancer is invasive or not.

The various reference levels in the above-described aspects may be calculated by determining the methylation state of a set of sites in patients who are known not to have breast cancer or are known to have DCIS, as appropriate for the respective embodiments. Accordingly, when a differential methylation level is determined using the same plurality of sites, deviation from the reference level indicates an additional evidentiary datum point supporting increasing the probability that the patient likely has breast cancer or invasive breast cancer.

A skilled person will understand that the specifics of the calculation used for generating a differential methylation level are not critical and various processes may be employed to generate these levels. All of them are within the scope of various embodiments of the invention.

Various embodiments of the invention rely on pluralities of CpG sites of various sizes. In various embodiments, the plurality of CpG sites may contain about 5, 10, 100, 1000, 5000 or more CpG sites.

Even among the CpG sites that have shown predictive power, different sites contribute different weightings to the overall predictive probability of the cancer status of a patient. Various embodiments of the invention calculate differential methylation levels based on various combinations of predictive CpG sites.

The predictive power of the sites may be quantified in various ways. As shown in FIGS. 1C, 5C, 8C and 11C, the deviation between the reference level and the differential methylation level for patients with DCIS or invasive breast cancer can be represented by non-metric multidimensional scaling (NMDS). Each site in the set will make a contribution to the ordinate discrimination in the NMDS. In some embodiments, the plurality of CpG sites includes one or more sites selected from Tables 3, 9, 15 or 21, in which the sites are ranked by the site's contribution to ordinate discrimination, as appropriate for the various embodiments.

The predictive power of CpG sites may also be quantified based on P-value, adjusted for False Discovery Rate (FDR). Tables 8, 14, 20 and 26 list the top 40 CpG sites by P-value. In various embodiments, the plurality of CpG sites in any of the above aspects or embodiments may include one or more sites selected from the Tables below, in particular, those that rank CpG sites by their contribution to NMDS or by p-value. In some embodiments, the plurality of CpG sites may include the top n sites or m of the top n sites from these Tables or other ordinal lists (wherein m and n are positive integers). All of the CpG sites recited herein may in various embodiments be included in the calculation.

In various embodiments, the plurality of CpG sites may include sites within genes that are hypermethylated among patients with DCIS relative to patients without evidence of breast cancer. Table 11 lists CpG sites according to this metric. In various embodiments, the plurality of CpG sites may include sites within genes that are hypermethylated among patients with invasive breast cancer relative to patients without evidence of breast cancer. Tables 17 lists CpG sites according to this metric. In various embodiments, the plurality of CpG sites may include sites within genes that are hypermethylated among patients with invasive breast cancer relative to patients with DCIS. Table 23 lists CpG sites according to this metric.

In various embodiments, the tumor sample may be any sample obtained from the patient that contains sufficient DNA such that the methylation states of the various CpG sites may be determined. In various embodiments, the sample may be a blood, saliva or tissue sample. In various embodiments, the sample may contain tumor cells. In various embodiments the sample may be a tumor tissue sample. In various embodiments, the sample may comprise peripheral blood mononuclear cells (PBMCs). In various embodiments, the sample may be enriched to contain predominantly or substantially exclusively one type of cell or tissue, by way of non-limiting example the sample may be processed to contain predominantly or exclusively PBMCs.

In various embodiments, the method further includes providing treatment for breast cancer to patients in whom cancer is indicated. This treatment will vary for patients with DCIS or invasive breast cancer. The treatment may include any form of standard of care treatment for the form of cancer indicated accepted by the extended medical community. This includes but is not limited to hormonal therapy, targeted therapy, immunotherapy, chemotherapy, radiation or surgery. In various embodiments, the treatment may be Evista (Raloxifene Hydrochloride) Keoxifene (Raloxifene Hydrochloride), Raloxifene Hydrochloride, Abitrexate (Methotrexate), Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Atezolizumab (Tecentriq) Capecitabine, Clafen (Cyclophosphamide), Cyclophosphamide, Cytoxan (Cyclophosphamide), Cisplatin, Carboplatin, Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ipilimumab (Yervoy), Ixabepilone, Ixempra (Ixabepilone), Kadcyla (Ado-Trastuzumab Emtansine), Kisqali (Ribociclib), Lapatinib Ditosylate, Letrozole, Megestrol Acetate, Methotrexate, Methotrexate LPF (Methotrexate), Mexate (Methotrexate), Mexate-AQ (Methotrexate), Neosar (Cyclophosphamide), Niraparib (Zejula), Nivolumab (Opdivo), Nolvadex (Tamoxifen Citrate), Olaparib (Lynparza), Paclitaxel, Paclitaxel Albumin-stabilized Nanoparticle Formulation, Palbociclib, Pamidronate Disodium, Perjeta (Pertuzumab), Pembrolizumab (Keytrundra), Pertuzumab, Ribociclib, Rucaparib (Rubraca), Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Vinblastine Sulfate, Xeloda (Capecitabine), Zoladex (Goserelin Acetate) and the like. In various embodiments, where carcinoma in situ is indicated, the treatment may include monitoring or in some embodiments be limited to monitoring the benign lesion to ensure that it has not progressed to an invasive form.

### Experimental Example

The invention is further described in detail by reference to the following experimental example. This example is provided for purposes of illustration only, and is not intended to be limiting unless otherwise specified. Thus, the invention should in no way be construed as being limited to the following example, but rather, should be construed to encompass any and all variations which become evident as a result of the teaching provided herein.

### Example 1

In order to test the hypothesis that methylation changes in circulating lymphocytes could discriminate between women with different stages of breast cancer and women without cancer, a sensitive platform to identify changes in specific CpG profiles in blood cells that correspond to disease states in breast cancer was used. Discrimination of methylation profiles of peripheral blood mononuclear cells (PBMCs) from women with breast cancer and healthy women is evident.

Overall, at a functional level, patterns of differential methylation can be traced back to pathways and genes that support hypotheses about the potential role of DNA methylation in the altered immunological response to breast cancer. These methylation signals are evident in blood even though they convey a distinct tumor activity signature. Even with a small pilot cohort (8 normal, 6 DCIS and 8 invasive breast cancer samples) epigenetic profiles that discriminate between normal and tumor blood profiles were recovered. As expected, the distribution of signals in the tumor samples represent the complexity of the disease; however, the patterns in blood cells from healthy women are relatively overlapping

ΔML was calculated as the summation of the difference in % MET scores for each CpG site within the defined region or structure being scored, averaged by the number of CpG sites present:

$$Δ ML = {\sum }_{i = first}^{last} [ {CpG}_{[ i ] [ Grp 1 ]} - {CpG}_{[ i ] [ Grp 2 ]} ] \qquad ( 1 )$$

where first and last CpG indexes are defined by the gene unit across which the summation score is being calculated. Thus, positive ΔML values indicate more methylation present in Grp1 and negative values indicate more methylation in Grp2.

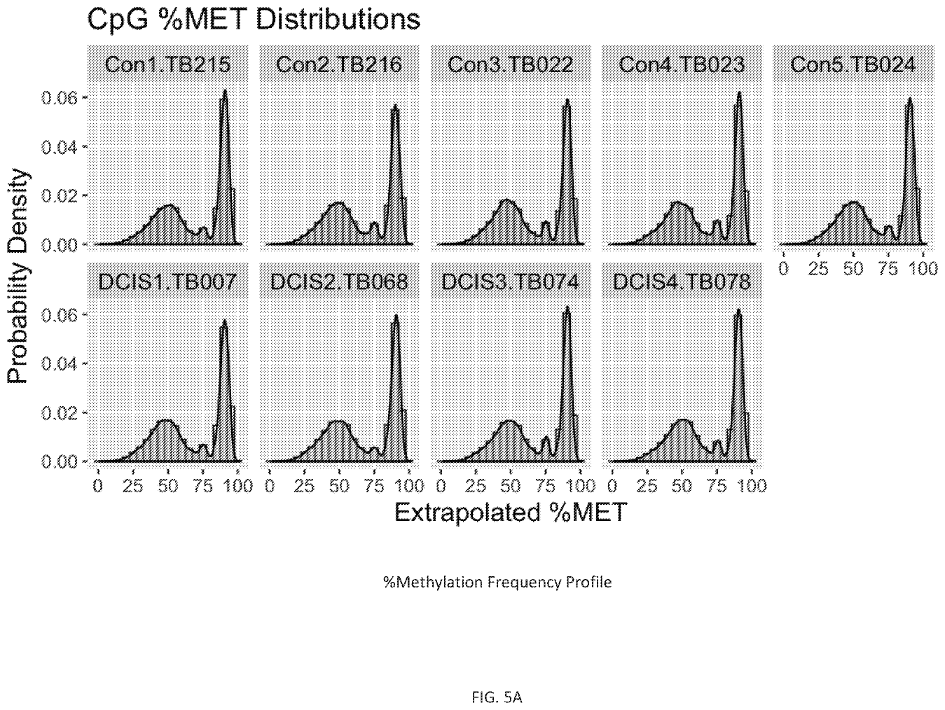

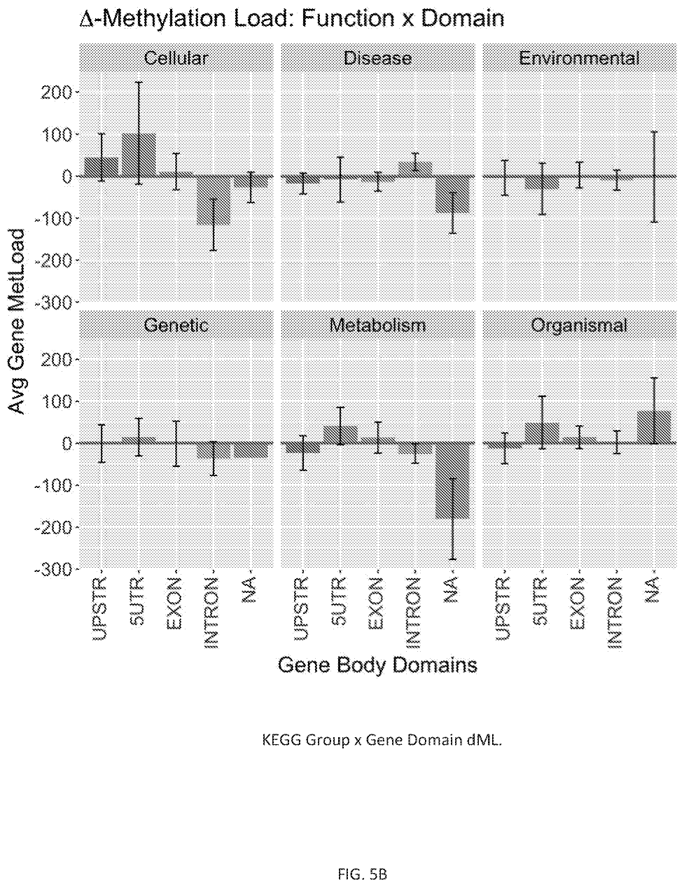

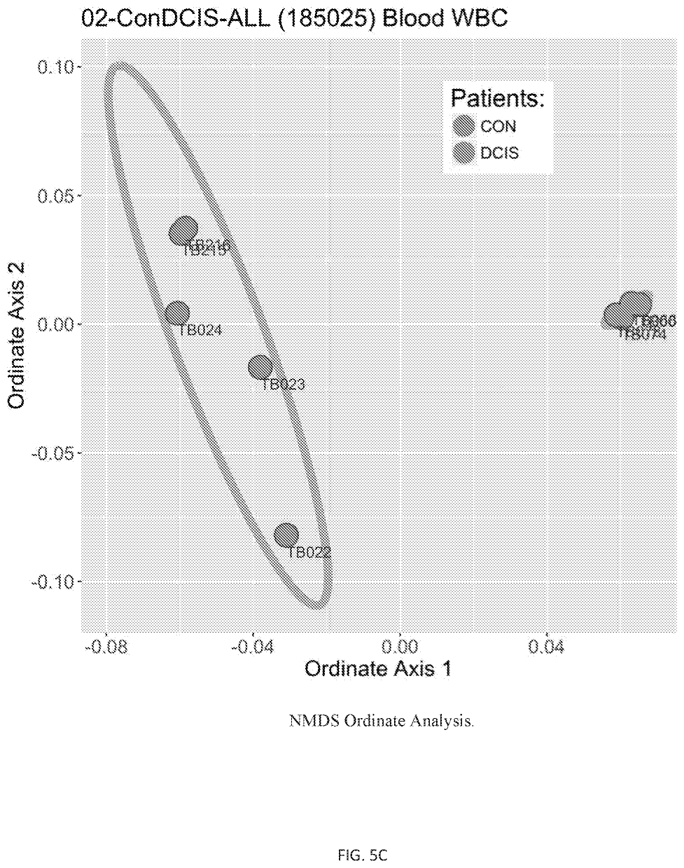

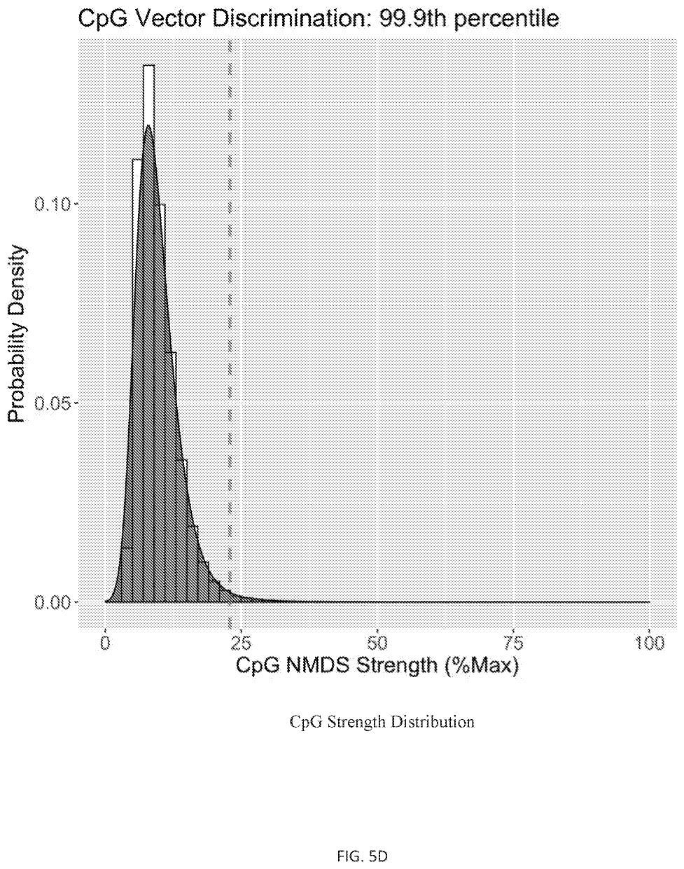

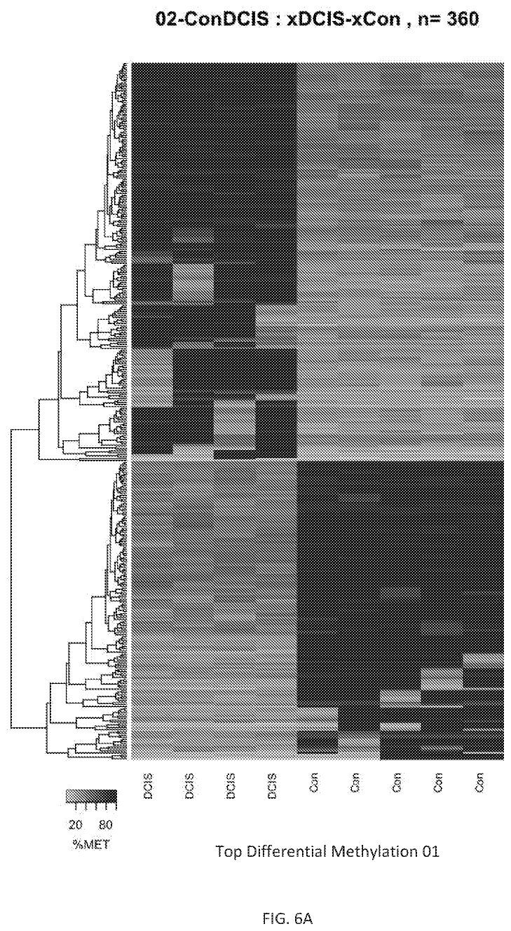

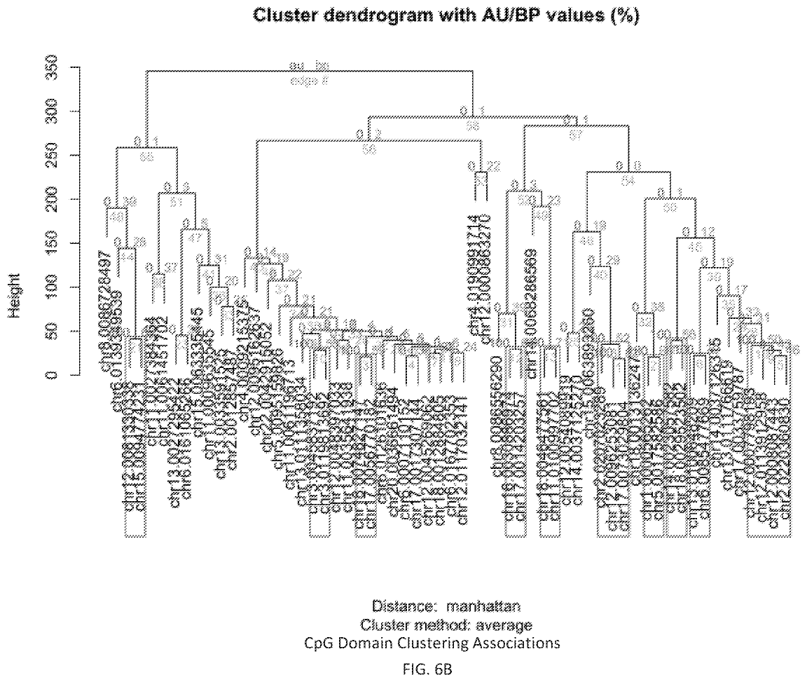

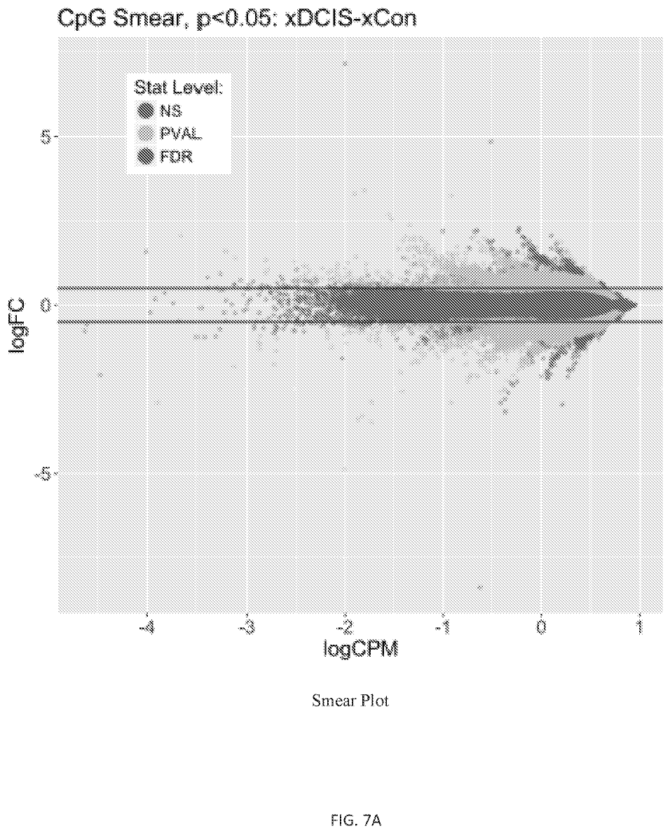

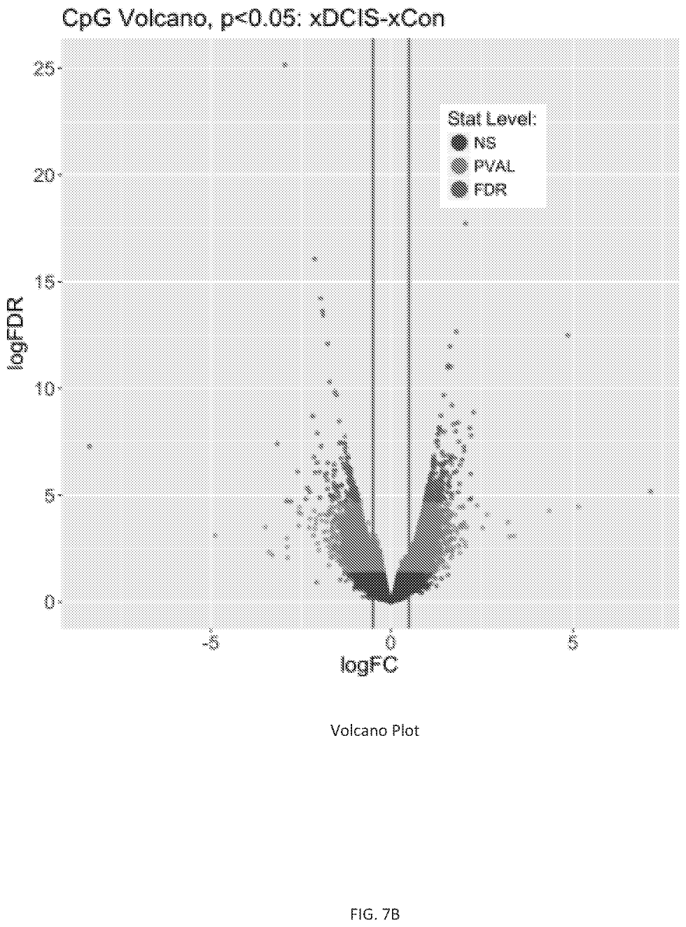

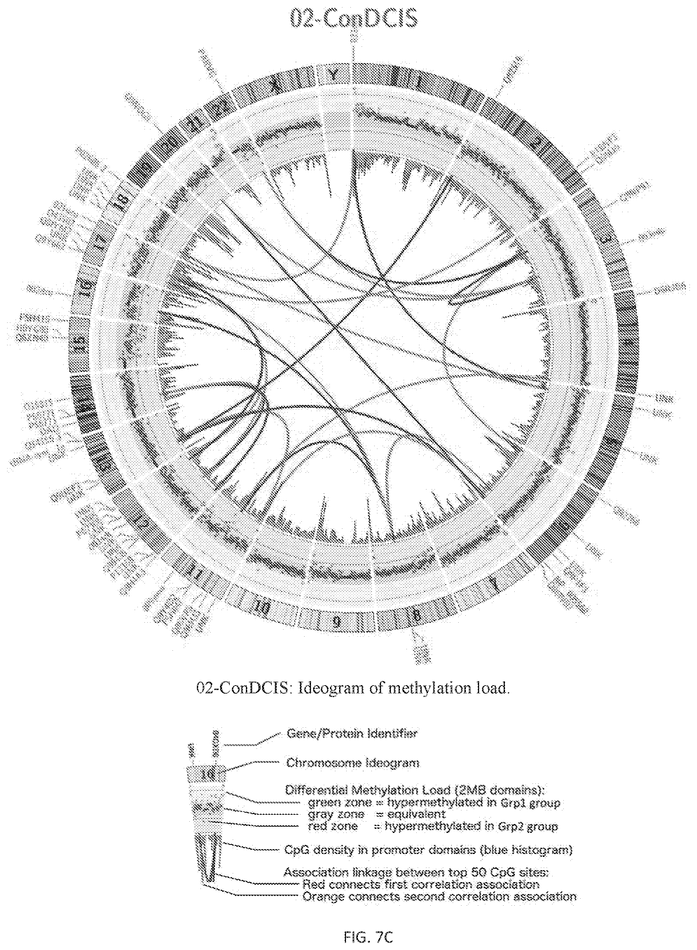

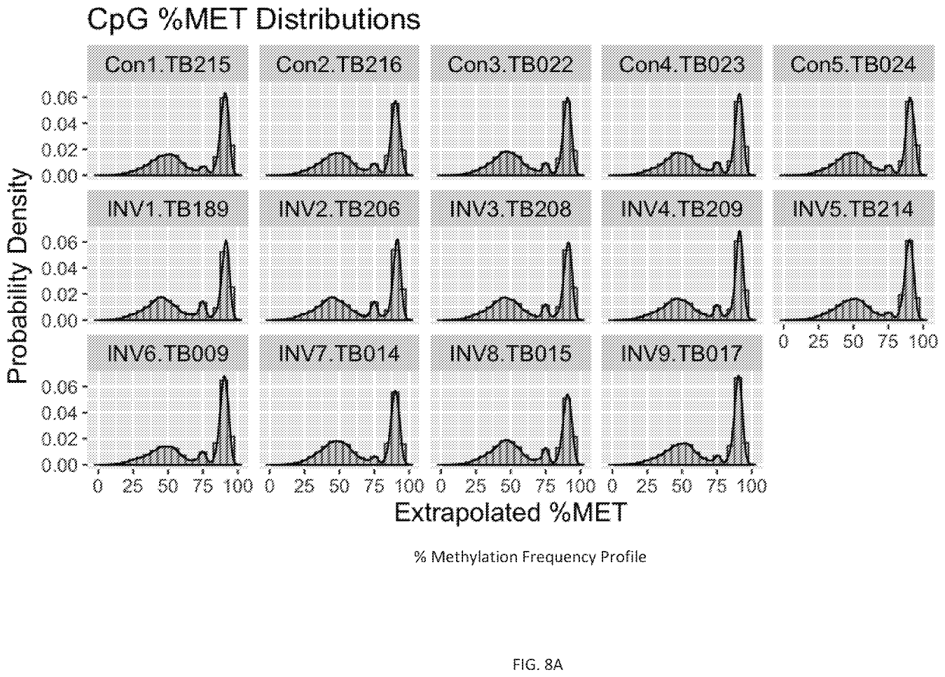

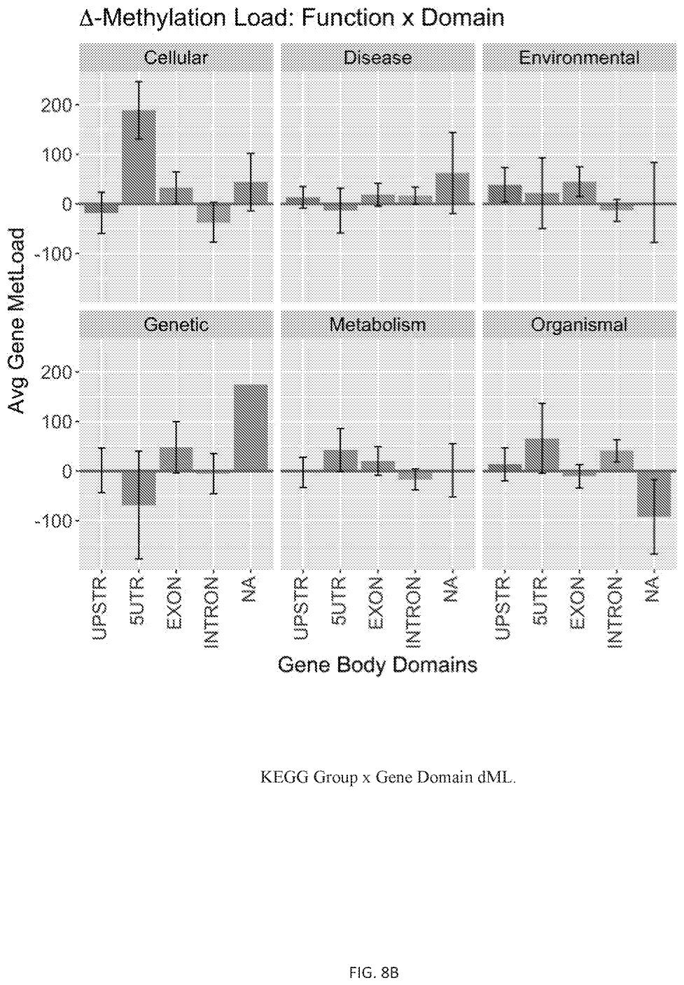

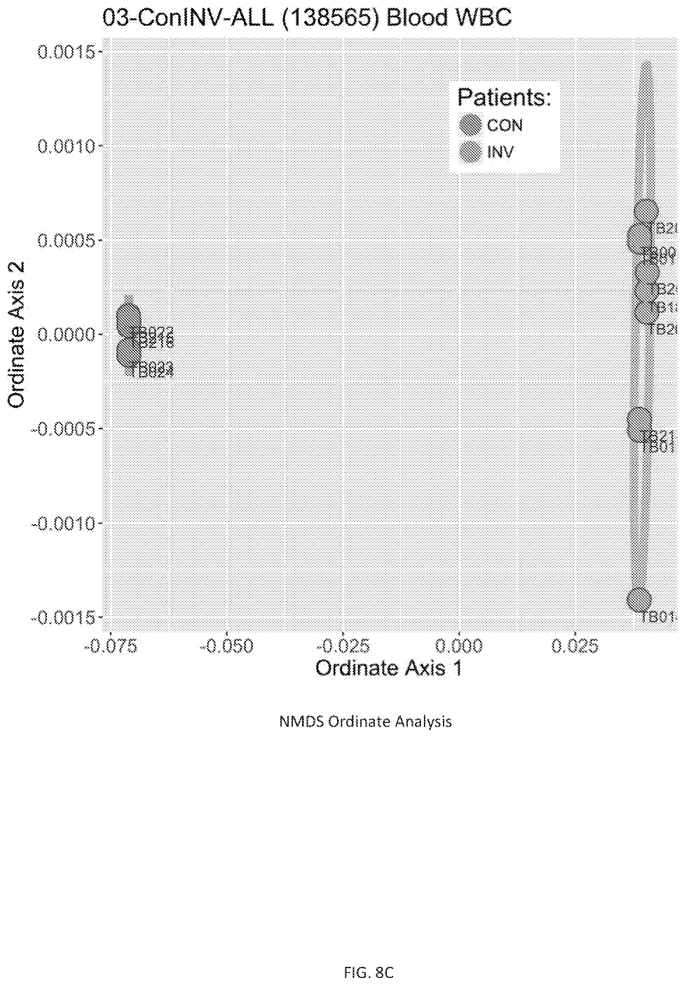

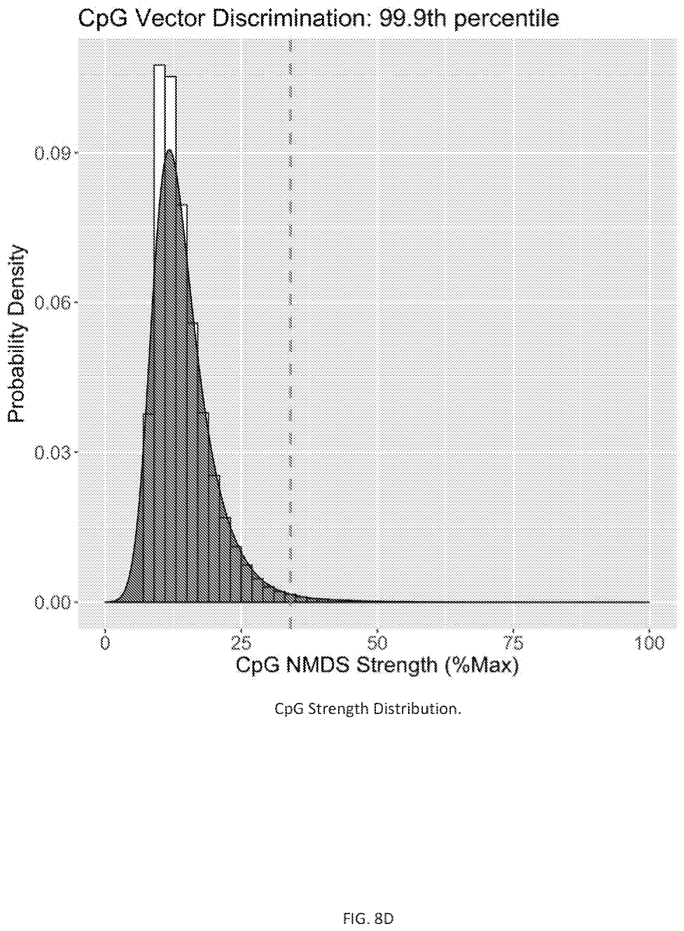

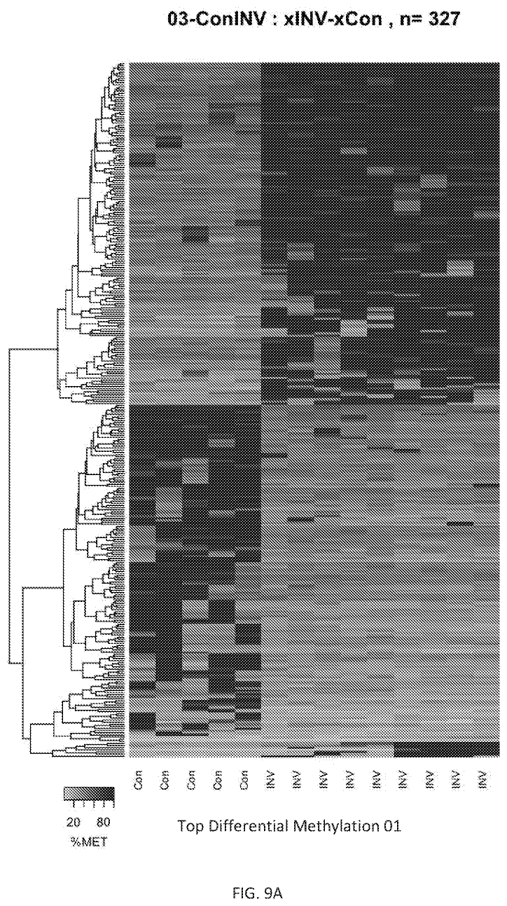

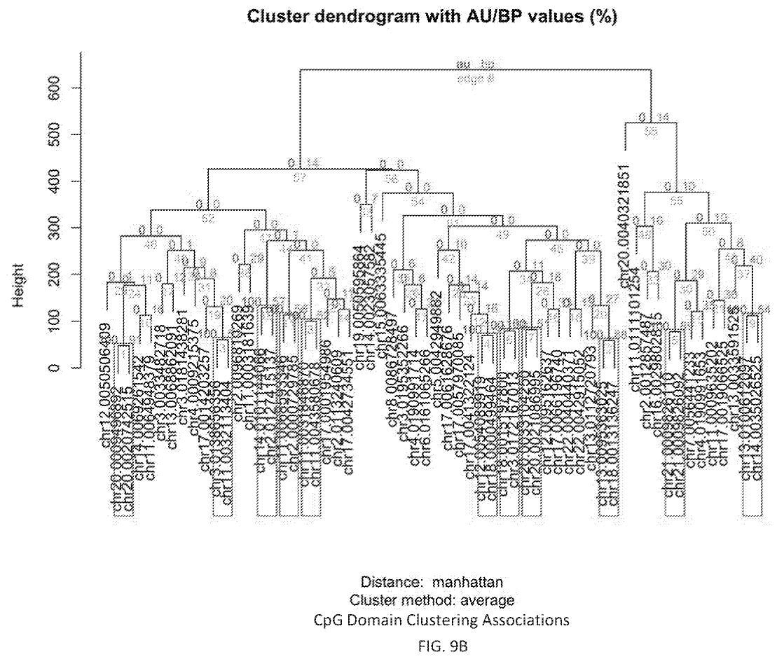

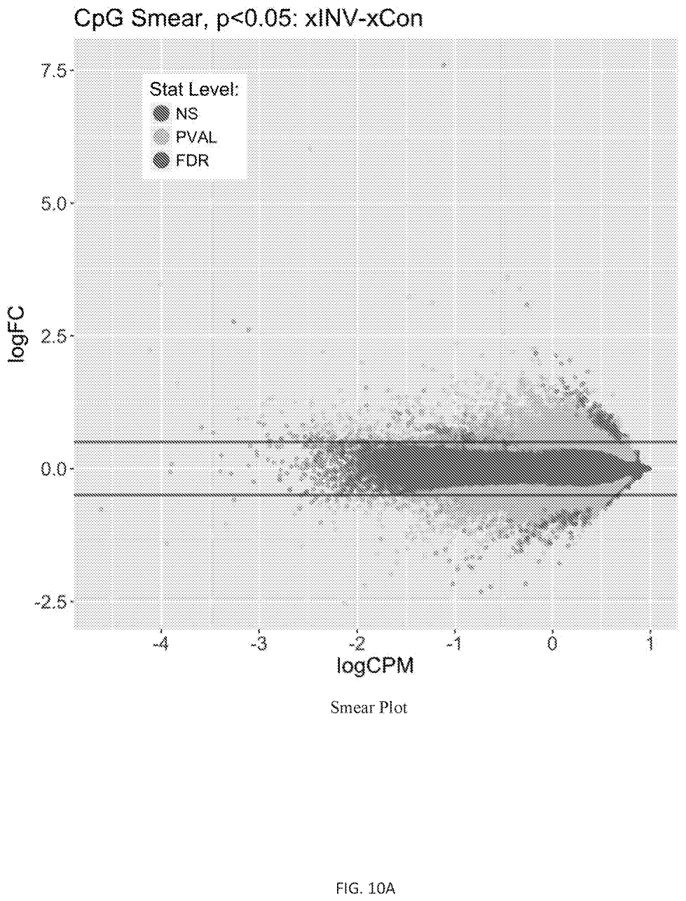

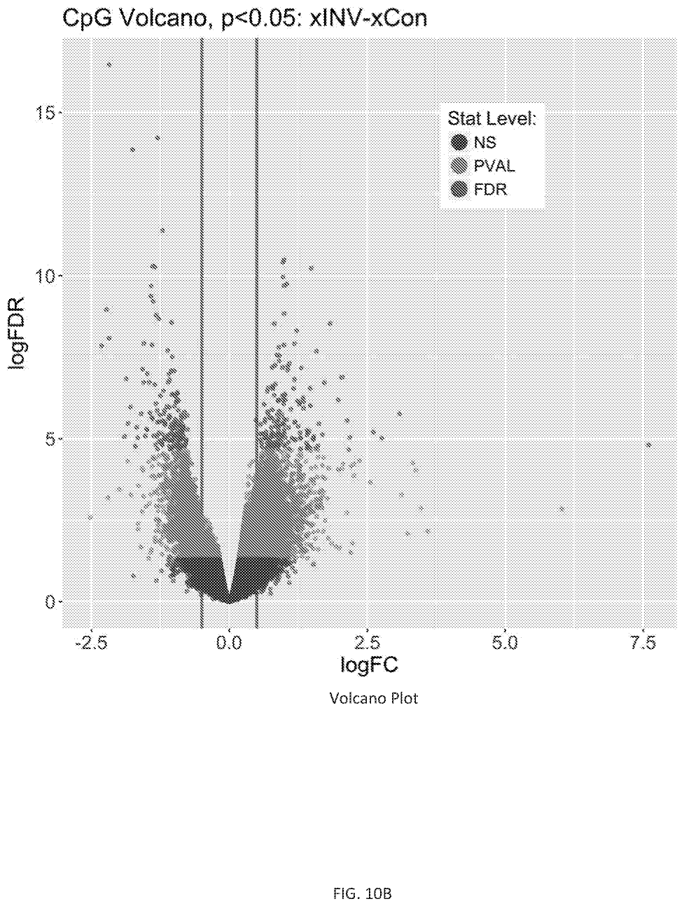

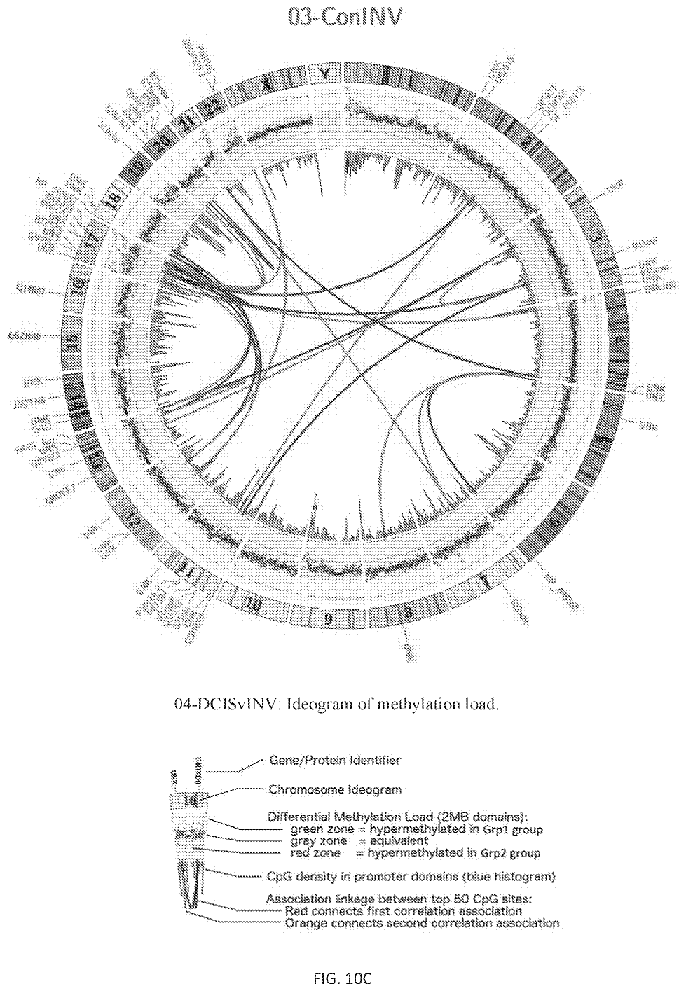

The pairwise analysis in Table 1 provides a direct contrast and allows for several graphical visualization plots to be generated these are presented in FIGS. 1-13. FIGS. 1-4 and Tables 3-8 illustrate the differences in epigenetic profile between patients with DCIS, patients with invasive breast cancer and patients without breast cancer (control) in a three-way analysis. FIGS. 5-7 and Tables 9-14 illustrate the differences in epigenetic profile between patients without breast cancer (control) and patients with DCIS. FIGS. 8-10 and Tables 15-20 illustrate the differences in epigenetic profile between patients with invasive breast cancer and control patients. FIGS. 11-13 and Tables 21-26 illustrate the differences in epigenetic profile between patients with DCIS and patients with invasive breast cancer.

As described below in Example 2, a blind study was conducted. The CpG sites used in that analysis are listed in Table 27. In various embodiments, the plurality of CpG sites may include one or more of the sites listed in Table 27.

Table 1 summarizes the group comparisons and the samples associated with each group.

TABLE 1
Defined Group Comparisons in this Analysis

| Code | | | |
|---|---|---|---|
| Project 1 | Grp1 | Grp2 | Samples |
| 04-ConDCIS | Healthy | DCIS | TB215, TB216, TB022, TB023, |
| | | | TB024, TB007, TB068, TB074, |
| | | | TB078 |
| 02-ConINV | Healthy | INVASIVE | TB215, TB216, TB022, TB023, |
| | | | TB024, TB189, TB206, TB208, |
| | | | TB209, TB214, TB009, TB014, |
| | | | TB015, TB017 |
| 03-DCIS-INV | DCIS | INVASIVE | TB007, TB068, TB074, TB078 |
| | | | TB189, TB206, TB208, TB209, |
| | | | TB214, TB009, TB014, TB015, |
| | | | TB017 |

* = Healthy Samples TB173 and TB190 are not included in these analyses because of their extreme departure in profile from the other Healthy samples. They are not similar in methylation pattern, just highly divergent at different CpG sites from the other Healthy samples.

### Example 2

To test the prognostic ability of epigenetic analysis of circulating lymphocytes, a blinded study was executed with an additional 30 samples. It is a separate cohort than the existing cohort, but it is a replication of the cohort with an experimental design that allowed for blind testing. The goal of the classification test was to examine blood samples from patients recently diagnosed with DCIS and assess whether there was a DNA methylation signature that could be indicative of the risk that the observed breast tissue lesion would be likely to become invasive or remain a benign cell type. For comparison, risk was determined based on independent pathologic criteria determined on lesions after surgical resection. Samples were scored as low risk (Cribroform subtype, no necrosis, low mitotic index) or high risk (Comedo or solid subtype, high mitotic index, necrosis).

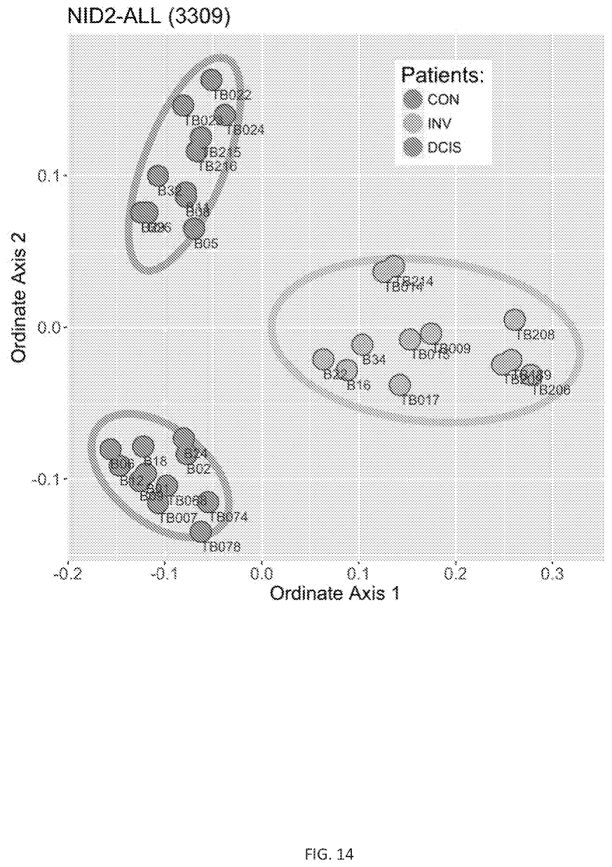

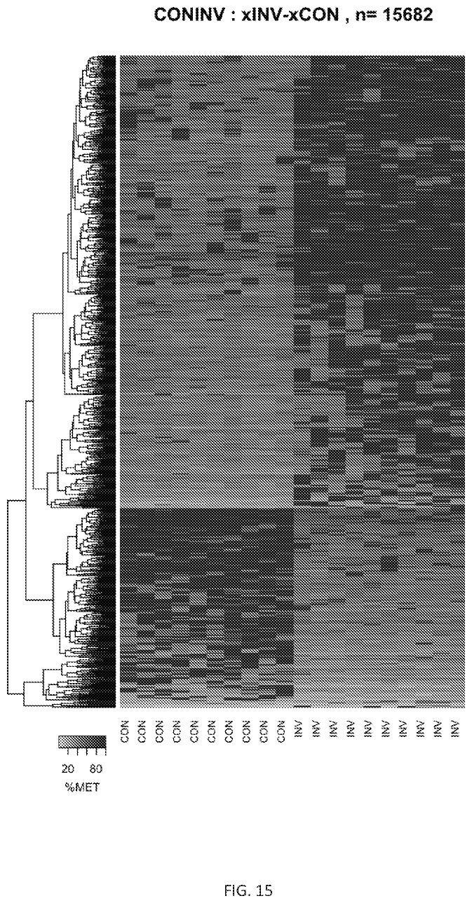

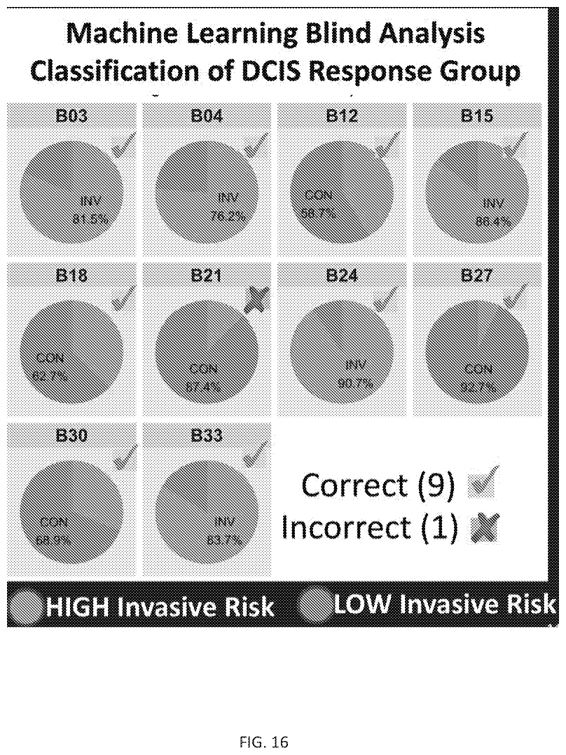

Nine out of the 10 blind samples were classified correctly in comparison to known pathologic criteria. To accomplish this, the control patients' and invasive patients' samples (blood DNA methylation profiles) were used to develop a discriminating classification routine. The herein disclosed methods were used to generate the statistical and algorithmic assets required to execute the classification calls on new, blind/unknown samples. The results are presented in FIGS. 14-16 and Table 2, below.

TABLE 2

| | | | | | RISK of invasiveness as | | |
|---|---|---|---|---|---|---|---|
| | | | | | determined by: | | |
| | | | | | | DNA Methylation | |
| Sample | | | | size | Pathologic | Profiling (THIS | |
| number | Pathological Subtype | Grade | necrosis | (mm) | Criteria | METHOD) | CORRECT |
| B03 | cribriform and comedo | 3 | yes | 25 | HIGH | HIGH | correct |
| B04 | solid and comedo | 3 | yes | 22 | HIGH | HIGH | correct |
| B12 | cribriformimg | 2 | no | 3 | LOW | LOW | correct |
| B15 | solid | 3 | yes | 10 | HIGH | HIGH | correct |
| B18 | cribriforming and | 1 | no | 15 | LOW | LOW | correct |
| | comedo | | | | | | |
| B21 | solid and cribriform | 3 | yes | 30 | HIGH | LOW | wrong |
| B24 | cribroform and solid | 3 | yes | 20 | HIGH | HIGH | correct |
| B27 | cribroform, | 1 | no | 2.5 | LOW | LOW | correct |
| | micropapillary | | | | | | |
| B30 | cribriform | 2 | no | 9 | LOW | LOW | correct |
| B33 | solid and cribriform | 2 | yes | 18 | HIGH | HIGH | correct |

### Rankings of Sites for the Three-Way Analysis

TABLE 3
Top 1000 CPG Sites. Rank Ordered listing of
CpG sites contributing the most to the ordinate
discrimination in the NMDS analysis.
CpG

chr21.0009826110
chr7.0056441233
chr4.0190991653
chr13.0027295422
chr20.0033104250
chr21.0009826092
chr12.0054089919
chr17.0014203257
chr8.0086728497
chr2.0113240609
chr12.0081330338
chr17.0019066525
chr6.0139349539
chr11.0027384364
chr6.0126661739
chr22.0042915052
chr11.0061451702
chr13.0100612097
chr4.0190991714
chr13.0112720793
chr11.0111101254
chr14.0023057582
chr13.0095363025
chr16.0029802815
chr18.0019284903
chr3.0181438281
chr22.0021615439
chr15.0045409777
chr13.0061989475
chr17.0033814506
chr13.0028364252
chr3.0120626543
chr16.0031227255
chr13.0050656618
chr18.0076738111
chr3.0033482718
chr12.0095162637
chr15.0094774231
chr16.0054323769
chr6.0161065266
chr5.0172671714
chr2.0096810269
chr3.0197392480
chr12.0008113542
chr4.0009215375
chr3.0149531388
chr14.0035008827
chr20.0021086995
chr14.0069261542
chr12.0113573025
chr5.0017582586
chr3.0138656356
chr14.0060976921
chr17.0038716331
chr15.0063334768
chr14.0019893202
chr1.0000565262
chr18.0000499464
chr3.0172167013
chr20.0056804010
chr12.0002862275
chr11.0016626136
chr9.0115823339
chr10.0135479593
chr2.0177014555
chr11.0070508612
chr11.0064684954
chr15.0041794125
chr6.0027247636
chr2.0018060121
chr9.0042368701
chr17.0061628676
chr18.0046477561
chr14.0101144066
chr12.0130662393
chr11.0009635202
chr17.0043129577
chr8.0086556290
chr14.0037126315
chr11.0057407074
chr17.0034497913
chr21.0010164209
chr3.0184302159
chr20.0055926272
chr18.0013136247
chr13.0033591525
chr14.0037133183
chr13.0068862091
chr6.0159572466
chr22.0040440371
chr20.0048553776
chr11.0068607299
chr4.0190990174
chr14.0094255732
chr19.0050595864
chr17.0057970085
chr16.0047007494
chr6.0010695540
chr11.0089713826
chr12.0106978718
chr6.0027248460
chr16.0023653028
chr20.0048730218
chr8.0086570466
chr2.0183731397
chr2.0000729785
chr12.0050506409
chr6.0002970717
chr22.0032341165
chr22.0023524377
chr22.0037680206
chr17.0012693967
chr14.0035026525
chr18.0030349745
chr11.0009635545
chr11.0119211657
chr18.0015197800
chr20.0062282831
chr3.0129118333
chr15.0032680456
chr20.0020742515
chr11.0003181639
chr6.0159525700
chr17.0041322124
chr5.0140767531
chr14.0058712032
chr22.0021213668
chr11.0064889237
chr18.0005238890
chr2.0042329510
chr11.0032113404
chr9.0099540553
chr12.0114839068
chr11.0043580678
chr11.0091598917
chr20.0009496892
chr13.0112723555
chr16.0055358884
chr8.0061430226
chr6.0035182463
chr11.0031846870
chr16.0058498594
chr17.0001954986
chr2.0091777959
chr13.0096205001
chr22.0031740075
chr5.0042991671
chr20.0060942639
chr20.0058515736
chr3.0149689478
chr6.0157744689
chr14.0038071393
chr11.0124734304
chr12.0117674458
chr6.0161034075
chr3.0038066016
chr21.0038068930
chr17.0066196740
chr17.0059534001
chr6.0026157100
chr18.0029523502
chr15.0100107315
chr11.0064948379
chr18.0077960570
chr11.0045433229
chr19.0036193143
chr22.0046299680
chr10.0003514879
chr12.0113573398
chr6.0034192386
chr6.0028505184
chr6.0026020848
chr17.0009548324
chr18.0044774814
chr9.0084561076
chr11.0069323971
chr9.0035603644
chr21.0009826075
chr9.0066457951
chr15.0101300081
chr3.0069134445
chr17.0036286187
chr12.0005539802
chr15.0075401874
chr12.0124339667
chr5.0042949882
chr17.0043250346
chr6.0026158708
chr20.0023017832
chr14.0068286569
chr12.0006641855
chr11.0046370207
chr2.0071127961
chr13.0112760981
chr6.0028864916
chr11.0118088293
chr15.0060297395
chr12.0040014292
chr6.0005999721
chr12.0127210936
chr12.0114886102
chr12.0055247863
chr22.0019746577
chr11.0124710147
chr5.0077254287
chr11.0066114505
chr12.0057856280
chr20.0056785111
chr22.0049764110
chr9.0132647804
chr12.0007798193
chr12.0088535308
chr13.0037574863
chr12.0000248545
chr20.0060310202
chr5.0092931810
chr16.0001877823
chr14.0064969377
chr12.0072665650
chr20.0062708881
chr3.0045837192
chr11.0041259310
chr3.0066023815
chr6.0085477669
chr13.0100648209
chr15.0063893260
chr3.0047323602
chr3.0129721027
chr11.0010316376
chr12.0045269062
chr21.0045721062
chr2.0230421733
chr4.0191007813
chr3.0197391931
chr14.0029235148
chr9.0133537741
chr21.0009826226
chr11.0118306730
chr3.0059466375
chr11.0071855196
chr3.0157824506
chr20.0040321851
chr22.0032807421
chr12.0095267668
chr13.0048612261
chr13.0095366027
chr2.0012857487
chr8.0022551030
chr11.0068593346
chr3.0190323321
chr17.0046723834
chr17.0043176656
chr3.0180319542
chr18.0020839973
chr16.0021512858
chr17.0056410100
chr14.0037130747
chr14.0072980894
chr14.0101925882
chr21.0009826146
chr6.0161037536
chr15.0028343785
chr6.0168110077
chr11.0047416016
chr12.0053719703
chr2.0055747731
chr20.0060693185
chr17.0058678996
chr15.0066547368
chr12.0132671272
chr18.0076481748
chr3.0128209966
chr17.0036719559
chr5.0063257095
chr17.0042634436
chr15.0070767299
chr3.0155945555
chr12.0108079458
chr12.0064215916
chr11.0031824327
chr20.0031034124
chr20.0002645380
chr6.0134210997
chr4.0190988866
chr11.0019546541
chr20.0043513977
chr15.0074365266
chr17.0042734551
chr22.0050752817
chr11.0044588016
chr19.0050038397
chr17.0045728221
chr8.0086570688
chr18.0027861099
chr6.0010381594
chr11.0133401937
chr3.0042845188
chr2.0080549750
chrX.0115003962
chr17.0075406104
chr14.0021093009
chr5.0171463182
chr3.0126006910
chr14.0097378133
chr22.0050742674
chr15.0089922792
chr12.0050616434
chr20.0043595994
chr14.0089290312
chr2.0131356252
chr11.0118794762
chr19.0042069923
chr2.0038978853
chr22.0039436523
chr20.0019984304
chr11.0001332393
chr3.0160939899
chr13.0022246503
chr12.0041720681
chr13.0112708614
chr2.0048648149
chr2.0087304475
chr12.0094361472
chr16.0030886974
chr22.0048541823
chr16.0001822579
chr13.0111766619
chr3.0126259929
chr11.0001474987
chr9.0110400086
chr7.0121048611
chr5.0151151794
chr2.0074056574
chr5.0140782367
chr17.0042072437
chr11.0000415553
chr14.0050101778
chr9.0134954595
chr17.0067577554
chr18.0057357804
chr16.0030671910
chr6.0027777950
chr5.0054527329
chr18.0055096263
chr12.0048358151
chr14.0069658819
chr20.0039995809
chr11.0047160760
chr11.0007695679
chr19.0016438474
chr11.0043604860
chr17.0056596237
chr20.0058090801
chr11.0134341441
chr18.0007371020
chr13.0021715169
chr13.0112330267
chr3.0049044952
chr12.0050453817
chr16.0089181384
chr20.0002853406
chr11.0031838687
chr18.0076737080
chr12.0014927550
chr6.0150184172
chr15.0101061098
chr6.0136571978
chr6.0011537594
chr11.0075111078
chr2.0127415137
chr20.0031330621
chr14.0065689243
chr13.0036052554
chr12.0096428593
chr19.0013267022
chr7.0024613735
chr3.0127634119
chr5.0003103410
chr17.0017654366
chr12.0046783625
chr9.0023824650
chr11.0096072761
chr12.0113313505
chr3.0122747539
chr12.0122675633
chr5.0115297957
chr18.0047721730
chr5.0069207752
chr15.0048625023
chr17.0059487528
chr12.0010874823
chr15.0023439042
chr12.0057630834
chr3.0137484002
chr15.0029864144
chr19.0059083815
chr3.0137480414
chr17.0079455749
chr3.0152552924
chr2.0020189294
chr15.0028344150
chr11.0067120835
chr3.0119422009
chr12.0048397033
chr6.0168079818
chr10.0135480431
chr20.0021083517
chr12.0050297405
chr22.0037663127
chr20.0021684369
chr13.0112760512
chr11.0104963181
chr2.0095691796
chr17.0080807535
chr16.0012667646
chr19.0042498208
chr15.0090743890
chr12.0000863710
chr3.0058554460
chr5.0013988181
chr5.0036745487
chr5.0174027285
chr20.0031352450
chr2.0217363364
chr11.0060929203
chr22.0017518146
chr18.0049868315
chr12.0103696281
chr6.0040995408
chr16.0000766221
chr12.0032908207
chr12.0054357015
chr11.0031821274
chr17.0018992459
chr19.0037761411
chr18.0046475810
chr12.0070637151
chr11.0019546133
chr6.0126101636
chr2.0204975304
chr5.0175960713
chr18.0013562830
chr17.0021023063
chr6.0131457089
chr3.0024537205
chr20.0031261476
chr17.0080292993
chr3.0008811437
chr22.0037663526
chr16.0030366829
chr14.0024564132
chr2.0016081660
chr2.0176946724
chr8.0065281465
chr11.0095657585
chr17.0008925911
chr15.0067814061
chr3.0071113928
chr12.0056223792
chr17.0046675549
chr11.0082611377
chr21.0009826287
chr15.0063335445
chr3.0188040907
chr12.0121018541
chr21.0011143747
chr5.0174155579
chr15.0068126000
chr3.0185977002
chr22.0020068641
chr12.0121564042
chr2.0045029060
chr5.0007851285
chr1.0000566317
chr13.0099064192
chr2.0039665281
chr6.0107386268
chr3.0119813692
chr21.0046130036
chr20.0061450707
chr17.0017876238
chr11.0124629895
chr15.0045927689
chr11.0046938767
chr17.0036287487
chr17.0001163539
chr15.0041794568
chr11.0066187812
chr3.0051704271
chr20.0032320496
chr20.0021690018
chr6.0010382800
chr20.0002821334
chr3.0107151212
chr11.0020619810
chr18.0074963218
chr17.0007465780
chr16.0057337873
chr15.0099559060
chr7.0096653354
chr20.0033542941
chr22.0042467277
chr3.0179182123
chr9.0000978453
chr3.0067705228
chr3.0195919117
chr15.0053076316
chr15.0089157895
chr2.0068695071
chr15.0066547713
chr12.0072332759
chr11.0017553236
chr19.0019976991
chr12.0097700243
chr11.0010229941
chr3.0009792106
chr20.0009049993
chr6.0001391144
chr18.0024131895
chr17.0048105091
chr12.0075724194
chr15.0072941453
chr2.0133426687
chr20.0062486416
chr11.0045101893
chr14.0024615469
chr17.0005342572
chr11.0116064679
chr13.0112187940
chr2.0039665069
chr7.0149917678
chr20.0003657392
chr11.0118087906
chr12.0004383507
chr7.0139184793
chr6.0144606507
chr22.0042678985
chr6.0031409291
chr12.0006450539
chr17.0046827033
chr15.0062359382
chr18.0060264186
chr15.0040799671
chr6.0027870991
chr18.0077304941
chr5.0037837160
chr15.0040886106
chr2.0070315802
chr11.0044327524
chr14.0069950620
chr3.0150805072
chr7.0087230305
chr13.0113295221
chr11.0067173421
chr7.0035301934
chr7.0121951055
chr21.0038740566
chr6.0027806422
chr5.0006745890
chr14.0037074363
chr15.0020774586
chr3.0147128690
chr15.0041221090
chr15.0089953564
chr17.0028257894
chr11.0002554562
chr17.0017696370
chr17.0073029804
chr5.0075698439
chr6.0151816054
chr2.0114262064
chr13.0098312933
chr6.0010694842
chr3.0111632475
chr6.0030313321
chr6.0100896338
chr11.0031827853
chr5.0141488834
chr20.0031126668
chr18.0011163944
chr15.0033487369
chr11.0070601917
chr20.0057227608
chr2.0043450965
chr3.0066025583
chr19.0045931076
chr12.0114877437
chr5.0058571658
chr17.0073268308
chr3.0147122989
chr17.0034748982
chr20.0034286460
chr12.0007055586
chr15.0075119454
chr5.0124536152
chr20.0039312811
chr9.0123555820
chr6.0053214211
chr8.0086727439
chr15.0045695003
chr2.0073152645
chr11.0006337183
chr6.0029691943
chr8.0008726877
chr12.0065066843
chr15.0068599199
chr18.0040051779
chr12.0133135361
chr2.0055459714
chr20.0002854843
chr15.0081475686
chr11.0125366224
chr12.0058088019
chr11.0065264490
chr9.0000972268
chr15.0040212392
chr15.0026965921
chr6.0071122748
chr11.0001913079
chr11.0117685841
chr17.0050236261
chr2.0171569107
chr17.0046655096
chr5.0077269177
chr17.0073106082
chr16.0066987650
chr14.0029255068
chr11.0100997702
chr6.0161034892
chr22.0019843427
chr3.0075471437
chr18.0011639297
chr2.0174831063
chr3.0192959361
chr14.0075077922
chr19.0039694617
chr22.0024952038
chr11.0124669378
chr19.0055553285
chr11.0001680823
chr20.0033064257
chr20.0029551109
chr15.0043415444
chr11.0031979880
chr20.0046601108
chr17.0042393959
chr17.0046696054
chr12.0121079347
chr22.0031740052
chr12.0124418736
chr11.0123008747
chr7.0039015708
chr3.0185912172
chr18.0045972953
chr16.0018043462
chr11.0064085532
chr20.0043514818
chr12.0122377340
chr20.0035274789
chr12.0002161319
chr17.0007590759
chr7.0005602782
chr20.0061554910
chr13.0112187284
chr19.0012163533
chr17.0073031426
chr12.0057506153
chr11.0108368623
chr15.0045410154
chr20.0021284593
chr14.0078227825
chr11.0000627511
chr3.0179753765
chr2.0239028866
chr13.0041364285
chr2.0148283585
chr12.0008087443
chr3.0014731336
chr18.0022126423
chr6.0085482109
chr17.0073629082
chr10.0104962224
chr11.0063687223
chr3.0061236525
chr14.0097050980
chr2.0177022661
chr12.0004227877
chr2.0232791921
chr12.0067663301
chr11.0093277585
chr2.0220361194
chr5.0137668011
chr22.0042487342
chr15.0099193744
chr12.0057118543
chr19.0054345439
chr5.0161495517
chr17.0007590939
chr12.0064233302
chr2.0120980577
chr6.0163837639
chr17.0033759787
chr17.0004047168
chr2.0045241408
chr5.0076010444
chr17.0019883369
chr17.0001000224
chr14.0091225099
chr13.0053030043
chr6.0036842708
chr13.0036045456
chr12.0006976124
chr12.0052626846
chr3.0136470077
chr12.0056323383
chr19.0046312992
chr6.0168842694
chr17.0019088754
chr13.0079018857
chr15.0058158361
chr20.0031128317
chr11.0065189464
chr20.0039319920
chr7.0027223194
chr8.0080740825
chr20.0042136963
chr5.0042949497
chr17.0016284098
chr6.0150284688
chr17.0018996904
chr20.0036661494
chr16.0030066605
chr21.0034603400
chr12.0110338921
chr11.0000308473
chr5.0088180118
chr11.0130319714
chr16.0005006250
chr18.0012884805
chr20.0022549081
chr12.0052414199
chr13.0041496017
chr13.0110761557
chr12.0054371989
chr15.0075495213
chr11.0070442560
chr14.0038678690
chr19.0000405313
chr18.0055104229
chr14.0090798926
chr17.0007190047
chr20.0003389329
chr3.0126195349
chr17.0057915773
chr7.0063361617
chr12.0067782441
chr12.0051476565
chr11.0065308645
chr14.0024611126
chr13.0112548733
chr4.0190943644
chr3.0107308392
chr11.0093707469
chr15.0047477411
chr16.0004364184
chr6.0026033650
chr20.0000591221
chr3.0193857514
chr7.0023507247
chr2.0010975677
chr12.0053732109
chr6.0157009112
chr19.0044576521
chr3.0024630223
chr20.0062609849
chr20.0061584448
chr16.0000216561
chr11.0100558372
chr22.0033040841
chr3.0157217384
chr9.0090256959
chr10.0047083620
chr5.0052286121
chr22.0037696640
chr9.0005339513
chr12.0056652330
chr3.0120278301
chr13.0077014601
chr11.0000382209
chr2.0202126440
chr15.0062599263
chr2.0192710945
chr2.0142523231
chr17.0008339890
chr19.0018060689
chr11.0002322072
chr16.0032211464
chr10.0103542667
chr5.0159849010
chr3.0006904601
chr12.0113489957
chr2.0046727530
chr11.0001676083
chr6.0010412348
chr15.0101421132
chr3.0148710056
chr12.0113542149
chr6.0100911125
chr17.0063553198
chr15.0040212792
chr12.0096253081
chr12.0132963622
chr11.0002190999
chr11.0064014492
chr3.0065583873
chr3.0147129333
chr22.0018846962
chr12.0007342661
chr8.0021645587
chr3.0120004419
chr12.0058158604
chr15.0045428732
chr3.0089164229
chr2.0091762598
chr11.0134525508
chr3.0072150129
chr7.0096642320
chr10.0102589581
chr2.0066809303
chr12.0006446797
chr6.0166078201
chr3.0050605386
chr6.0157390053
chr13.0026624812
chr11.0064757787
chr2.0241151076
chr12.0051442996
chr14.0101963435
chr2.0172952158
chr11.0012696865
chr9.0137354379
chr22.0049828000
chr14.0060975811
chr12.0129282241
chr2.0080300303
chr20.0060100951
chr11.0073490719
chr2.0157199292
chr12.0054338819
chr15.0093634307
chr12.0115122028
chr15.0032829086
chr17.0021183474
chr11.0047600191
chr18.0035147994
chr3.0019188810
chr19.0058520890
chr12.0000679392
chr22.0021922517
chr7.0131229865
chr22.0050455644
chr6.0079793340
chr17.0019028901
chr20.0034682243
chr14.0052746616
chr12.0054806459
chr5.0157965751
chr22.0047784689
chr12.0057633739
chr9.0000973262
chr15.0020733814
chr2.0157293096
chr17.0077775218
chr12.0054360615
chr6.0146350400
chr9.0035972387
chr11.0126033819
chr9.0093864177
chr14.0021091166
chr12.0093967107
chr5.0174402771
chr17.0045809660
chr21.0036258497
chr18.0074827147
chr11.0118566515
chr11.0117865334
chr11.0031830385
chr3.0048884826
chr17.0078944136
chr8.0086736710
chr2.0175616150
chr18.0044556314
chr14.0105433783
chr13.0024828484
chr20.0050283865
chr12.0066276259
chr18.0031803791
chr21.0045147607
chr11.0003185764
chr11.0125011371
chr11.0107328843
chr3.0128565855
chr12.0056512395
chr16.0057918264
chr12.0119616913
chr5.0172669936
chr7.0079764260
chr14.0069262002
chr15.0059225979
chr5.0087988624
chr20.0055956988
chr17.0059475373
chr15.0033023727
chr6.0003742885
chr16.0003152427
chr18.0055106910
chr13.0031248541
chr15.0063570562
chr3.0038081164
chr19.0034397576
chr21.0038083060
chr6.0050679720
chr11.0058975476
chr6.0036410268
chr3.0185653023
chr11.0036237395
chr17.0001993730
chr12.0000584675
chr12.0054391688
chr16.0032986491
chr18.0060028152
chr9.0033082990
chr11.0089518999
chr2.0073518897
chr3.0119014121
chr6.0073330966
chr10.0023479600
chr7.0137693710
chr16.0049891504
chr16.0067876966
chr5.0153126277
chr3.0101232351
chr15.0096596722
chr13.0027580970
chr15.0099104915
chr3.0018464995
chr11.0116114906
chr6.0143832776
chr20.0060607587
chr11.0064948716
chr6.0075916565
chr2.0214149450
chr17.0004047257
chr16.0087889984
chr8.0067025063
chr8.0007114712
chr15.0024123323
chr3.0120461953
chr9.0019231893
chr16.0029674073
chr14.0033401547
chr16.0014728089
chr16.0086884396
chr17.0077216743
chr17.0046693239
chr20.0055532410
chr12.0006277039
chr12.0014956526
chr14.0093132774
chr5.0134368249
chr11.0061463399
chr3.0075690414
chr2.0090458454
chr22.0045018332
chr9.0120178198
chr2.0149285701
chr16.0086538066
chr12.0058814470
chr6.0050804133
chr7.0100465214
chr3.0068057166
chr12.0007071780
chr15.0078252068
chr12.0069036581
chr17.0034754080
chr17.0035298681
chr13.0088327000
chr14.0037428846
chr12.0130529772
chr11.0102702295
chr6.0036089235
chr13.0113343518
chr17.0039464813
chr6.0118229764
chr18.0067957351
chr12.0125005873
chr6.0038683255
chr17.0018759674
chr7.0104584531
chr15.0090686530
The CpG list has been filtered for functional pathway assignments. CpGs in hypothetical genes or unknown gene domains with unannotated functions were not included in this hard-copy listing, but all CpGs and their scores are retained in the database and are available for further analyses.

TABLE 4
Top 40 Hypermethylated Genes in Grpl.
Genes are presented with PFAM and RefSeq name designations as well as KEGG pathway associations.

| MethyLoad | UniProt | HUGO | Reactome |
|---|---|---|---|
| 6843.2 | C9JJ H3 | unk | Ub-specific.processing.proteases |
| 6050.5 | Q8NGC1 | OR11G2 | Olfactory.Signaling.Pathway |
| 5274.3 | Q8NGA5 | OR10H4 | Olfactory.Signaling.Pathway |
| 5238.1 | Q8NH85 | OR5R1 | Olfactory.Signaling.Pathway |
| 5203.8 | P10412 | HIST1H1E | Formation.of.Senescence-Associated.Heterochromatin.Foci. |
| 4872.9 | O95222 | OR6A2 | Olfactory.Signaling.Pathway |
| 4263.0 | Q96RD0 | OR8B2 | Olfactory.Signaling.Pathway |
| 4263.0 | Q8NHB1 | OR2V1 | Olfactory.Signaling.Pathway |
| 4125.6 | Q8NGC3 | OR10G2 | Olfactory.Signaling.Pathway |
| 3767.1 | Q8NGK5 | OR52M1 | Olfactory.Signaling.Pathway |
| 3059.6 | Q8NGM9 | OR8D4 | Olfactory.Signaling.Pathway |
| 2705.2 | Q8NGP3 | OR5M9 | Olfactory.Signaling.Pathway |
| 2613.2 | P01563 | IFNA2 | TRAF6.mediated. IR F7.activation |
| 2611.5 | Q969M2 | GJA10 | Gap.junction.assembly |
| 2552.5 | P11021 | HSPA5 | Antigen.Presentation:.Folding,.assembly.and.peptide.load |
| 2481.8 | Q7L3B6 | CDC37L1 | Platelet.degranulation. |
| 2356.3 | Q9P032 | NDUFAF4 | Complex.I.biogenesis |
| 2339.5 | Q8NGR5 | OR1L4 | Olfactory.Signaling.Pathway |
| 2261.1 | Q15615 | OR4D1 | Olfactory.Signaling.Pathway |
| 2251.5 | Q8NGS6 | OR13C3 | Olfactory.Signaling.Pathway |
| 2203.3 | Q8NH73 | OR4S2 | Olfactory.Signaling.Pathway |
| 2195.0 | O95006 | OR2F2 | Olfactory.Signaling.Pathway |
| 2123.1 | Q8NGY2 | OR6K2 | Olfactory.Signaling.Pathway |
| 2109.2 | Q8NH18 | OR5J2 | Olfactory.Signaling.Pathway |
| 2107.0 | Q53H54 | TRMT12 | Synthesis.of.wybutosine.at.G37.of.tRNA(Phe) |
| 2048.2 | P14652 | HOXB2 | Activation.of.anterior.HOX.genes.in.hindbrain.developmen |
| 2032.4 | Q15388 | TOMM20 | Ub-specific.processing.proteases |
| 2009.0 | Q3LFD5 | USP41 | ISG15.antivira.mechanism |
| 1980.8 | Q8NGD1 | OR4N2 | Olfactory.Signaling.Pathway |
| 1952.2 | Q8NG41 | NPB | Peptide.ligand-binding.receptors |
| 1936.0 | Q8NGT7 | OR2A12 | Olfactory.Signaling.Pathway |
| 1918.2 | Q5JQS5 | OR2B11 | Olfactory.Signaling.Pathway |
| 1856.2 | P59535 | TAS2R40 | G.alpha.(i).signaling.events |
| 1794.2 | Q8NGA6 | OR10H5 | Olfactory.Signaling.Pathway |
| 1780.2 | P02533 | KRT14 | Type.I.hemidesmosome.assembly |
| 1777.8 | Q8NGV0 | OR2Y1 | Olfactory.Signaling.Pathway |
| 1740.8 | Q13145 | BAMBI | Downregulation.of.TFG-beta.receptor.signaling |
| 1720.9 | P01567 | IFNA7 | TRAF6.mediated.IRF7.activation |
| 1692.5 | Q8NGX0 | OR11L1 | Olfactory.Signaling.Pathway |
| 1675.3 | Q5H8A3 | NMS | G.alpha.(q).signaling.events |

The gene list has been filtered for functional pathway assignments.
Hypothetical genes or genes with unannotated functions were not included in this hard-copy listing, but all genes and their scores are retained in the database and are available for further analyses.

TABLE 5
Top 40 Hypermethylated Genes in Grp2.
Genes are presented with PFAM and RefSeq name designations as well as KEGG pathway associations.

| MethyLoad | UniProt | HUGO | Reactome |
|---|---|---|---|
| −7184.6 | Q96R19 | TAAR9 | G.alpha.(s).signalling.events |
| −5915.6 | Q8NGS3 | OR1J1 | Olfactory.Signaling.Pathway |
| −5203.1 | O60404 | OR10H3 | Olfactory.Signaling.Pathway |
| −4655.8 | Q8NGE9 | OR9Q2 | Olfactory.Signaling.Pathway |
| −4459.2 | Q9H1M4 | DEFB127 | Defensins |
| −4048.5 | Q9NV35 | NU DT15 | Phosphate.bond.hydrolysis.by.NUDT.proteins |
| −3739.1 | Q9NZP0 | OR6C3 | Olfactory.Signaling.Pathway |
| −3728.2 | Q6IF42 | OR2A2 | Olfactory.Signaling.Pathway |
| −3590.9 | Q96L21 | RPL1OL | Nonsense.Mediated.Decay.(NMD).independent.of.the.Exon.Ju |
| −3497.5 | Q58HT5 | AWAT1 | Wax.biosynthesis |
| −3469.1 | Q9H2C8 | ORS1V1 | Olfactory.Signaling.Pathway |
| −3188.0 | A6NL26 | OR5B21 | Olfactory.Signaling.Pathway |
| −3047.1 | Q8IXM3 | MRPL41 | Mitochondrial.translation.initiation |
| −2983.7 | P09681 | GIP | G.alpha.(s).signalling.events |
| −2873.1 | Q6PGQ7 | BORA | Regulation.of.PLK1.Activity.at.G2/M.Transition |
| −2870.2 | Q8NH60 | OR52J3 | Olfactory.Signaling.Pathway |
| −2831.0 | Q96PE6 | ZIM3 | Generic.Transcription.Pathway |
| −2756.9 | Q9HDD0 | HRASLS | Acyl.chain.remodelling.of.PE |
| −2740.3 | P12104 | FABP2 | Hormone-sensitive.lipase.(HSL)-mediated.triacylglycerol. |
| −2710.7 | P00326 | ADH1C | Ethanol.oxidation |
| −2541.0 | Q7Z7M8 | B3GNT8 | O-linked.glycosylation.of.mucins |
| −2535.7 | A6NP11 | ZNF716 | Generic.Transcription.Pathway |
| −2444.0 | Q8NGH7 | OR52L1 | Olfactory.Signaling.Pathway |
| −2435.9 | Q6VVB1 | NHLRC1 | Myoclonic.epilepsy.of.Lafora |
| −2346.7 | P82930 | MRPS34 | Mitochondrial.translation.initiation |
| −2324.1 | O76100 | OR7A10 | Olfactory.Signaling.Pathway |
| −2194.5 | Q8NG97 | OR2Z1 | Olfactory.Signaling.Pathway |
| −2181.1 | Q8NGE5 | OR10A7 | Olfactory.Signaling.Pathway |
| −2157.6 | Q6ZYL4 | GTF2H5 | Dual.incision.in.TC-NER |
| −2146.1 | P62310 | LSM3 | mRNA.Splicing.-.Major.Pathway |
| −2017.1 | Q8NG99 | OR7G2 | Olfactory.Signaling.Pathway |
| −2065.8 | Q9NUP1 | BLOC1S4 | Golgi.Associated.Vesicle.Biogenesis |
| −2007.4 | Q9BXT5 | TEX15 | Meiotic.recombination |
| −1971.8 | Q6UXV4 | APOOL | Platelet.degranulation. |
| −1963.6 | P22680 | CYP7A1 | Synthesis.of.bile.acids.and.bile.salts.via.27-hydroxycho |
| −1933.4 | P30939 | HTR1F | G.alpha.(i).signaling.events |
| −1909.8 | P81277 | PRLH | Peptide.ligand-binding.receptors |
| −1901.6 | Q8NGS1 | OR1J4 | Olfactory.Signaling.Pathway |
| −1901.1 | Q30KP8 | DEFB136 | Defensins |
| −1798.3 | Q9NYY3 | PLK2 | TP53.regulates.transcription.of.additional.cell.cycle.ge |

The gene list has been filtered for functional pathway assignments.
Hypothetical genes or genes with unannotated functions were not included in this hard-copy listing, but all genes and their scores are retained in the database and are available for further analyses.

TABLE 6
Top 30 Hypermethylated Pathways in Grp1.
Pathways are rank ordered by methylation load score (ΔML) and
include a list of the top 10 scoring genes contributing to the
pathways load value. The gene list is organized
in decreasing numerical order of the individual gene ΔML scores

| MethyLoad | Pathway | Genes |
|---|---|---|
| 1729.2 | Trypotophan.metabolism | unk |
| 1518.0 | Rheumatoid.arthritis | Q16552 |
| 1325.7 | B.cell.receptor.signaling.path | Q9UN19 |
| 1101.5 | Streptomycin.biosynthesis | 095455 |
| 1037.6 | Arrhythmogenic.right.ventricul | P17302 |
| 869.7 | Basal.cell.carcinoma | P12643 |
| 794.6 | Pantothenate.CoA.biosynthesis | 095497 |
| 775.9 | Metabolism.xenobiotics.by.cyto | 095154 |
| 745.9 | Autoimmune.thyroid.disease | A0A0R4J2F0 |
| 720.6 | Chloroalkane.chloroalkene.degr | P30837 |
| 679.7 | Neurotrophin.signaling.pathway | unk |
| 574.6 | Aldosteroneregulated.sodium.re | unk |
| 543.8 | Bisphenol.degradation | unk |
| 525.6 | Intestinal.immune.network.for | P01833 |
| 509.1 | Amyotrophic.lateral.sclerosis. | E7ESP9, P04040, |
| | | P12036 |
| 498.8 | Phagosome | Q5KU26 |
| 462.7 | Tight.junction | Q15334, P51153, |
| | | P20337 |
| 442.6 | Fatty.acid.elongation | Q9GZR5, Q9HB03 |
| 433.4 | Reninangiotensin.system | unk |
| 418.1 | Circadian.entrainment | P48039, B7ZA25 |
| 413.5 | Melanogenesis | P14679, Q01726 |
| 406.7 | Proximal.tubule.bicarbonate.re | P49448 |
| 398.7 | Glutathione.metabolism | Q96SL4 |
| 391.3 | Vasopressinregulated.water.rea | unk |
| 370.0 | Leishmaniasis | P01583, P12314, |
| | | P49006, P23458 |
| 356.6 | Collecting.duct.acid.secretion | P51164, P02730 |
| 349.6 | Pancreatic.secretion | P32238 |
| 348.5 | Endocrine.other.factorregulate | Unk |
| 339.3 | SNARE.interactions.in.vesicula | O95183, O75558 |
| 339.1 | Phenylpropanoid.biosynthesis | P30041 |

Gene order within each pathway is determined by decreasing methylation load scores ΔML).
The first gene in the list contributes the most to the pathway methylation score.

TABLE 7
Top 30 Hypermethylated Pathways in Grp2.
Pathways are rank ordered by methylation load score (ΔML) and
include a list of the top 10 scoring genes contributing to the
pathways load value. The gene list is organized
in decreasing numerical order of the individual gene ΔML scores.

| MethyLoad | Pathway | Genes |
|---|---|---|
| −1238.0 | Malaria | P07996, Q12918 |
| −1148.7 | Cell.cycle.yeast | unk |
| −1096.8 | Twocomponent.system | unk |
| −1091.9 | Meiosis.yeast | Q14566 |
| −1036.6 | Inositol.phosphate.metabolism | Q8NFU5 |
| −943.8 | Taurine.hypotaurine.metabolism | Q16878, Q96SZ5 |
| −917.7 | Synaptic.vesicle.cycle | Q7Z7G2 |
| −898.0 | Antigen.processing.presentatio | unk |
| 864.1 | JakSTAT.signaling.pathway | unk |
| −792.4 | Phototransduction.fly | unk |
| −785.6 | Valine.leucine.isoleucine.degr | unk |
| −785.0 | Atrazone.degradation | unk |
| −740.7 | Thyroid.cancer | Unk |
| −676.8 | Toluene.degradation | Q96DG6 |
| −669.8 | Natural.killer.cell.mediated.c | Q9BZM5, Q9BZM6 |
| −666.2 | Apoptosis | 014249 |
| −653.2 | Lipoic.acid.metabolism | unk |
| −624.4 | Base.excision.repair | unk |
| −574.8 | Nonhomologous.endjoining | unk |
| −549.0 | Glycosphingolipid.biosynthesis | 043505, P19526, |
| | | Q9Y231, Q8NDV1 |
| −539.4 | Glycosylphosphatidylinositolan | unk |
| −492.8 | Sulfur.relay.system | Q7Z7A3, O95396 |
| −481.5 | Viral.carcinogenesis | A0A0A0MSJ9, |
| | | 014839, P51679 |
| −461.6 | Fructose.mannose.metabolism | Q9NQ88 |
| −423.4 | Mismatch.repair | unk |
| −412.9 | Insulin.secretion | P09681, P43220, |
| | | Q8TDV5, 014842 |
| −396.6 | Basal.transcription.factors | P13984, Q15545, |
| | | Q81ZX4 |
| −392.3 | Peroxisome | Q567V2, Q9BY49, |
| | | P47989, P56589, |
| | | P21549, 095822, |
| | | Q9UBJ2, A8MXV4, |
| | | Q15126, Q9UJM8 |
| −382.9 | Terpenoid.backbone.biosynthesi | 075844, Q9BXS1 |
| −363.1 | Methane.metabolism | P05062 |

Gene order within each pathway is determined by decreasing methylation load scores (ΔML).
The first gene in the list contributes the most to the pathway methylation score.

TABLE 8
Top 40 CpG Sites by P-value.
Rank ordered listing of CpG sites in known UniProt
genes are sorted by P-values adjusted for false discovery rate (FDR) threshold.
Site-specific dispersion was estimated to equalize CpG variances. A Likelihood
Ratio Test was used with a defined one-way ANOVA model for pairwise tests.

| | | FDR adj | | | |
|---|---|---|---|---|---|
| CpG Site | IogFC | P-val | Response | Gene | Description |
| chr9.0101559153 | −1.26 | 2.38e−12 | 0.713 | ENSG00000165138 | — |
| chr2.0113240609 | −2.18 | 2.19e−06 | −0.42 | TTL | tubulin tyrosine |
| | | | | | ligase |
| chr12.0127210936 | 1.47 | 8.13e−05 | −0.32 | ENSG00000189238 | NP.001273148 |
| chr11.0067173421 | 1.18 | 0.000 | 0.111 | F5H037 | — |
| chr11.0065143966 | −0.76 | 0.000 | 0.729 | ENSG00000162241 | NP.001265180 |
| chr6.0010695540 | 1.65 | 0.000 | −0.42 | PAK1IP1 | PAK1 interacting |
| | | | | | protein 1 |
| chr3.0184302159 | 1.57 | 0.000 | −0.58 | H7C2X0 | |
| chr22.0039813308 | −1.04 | 0.001 | 0.669 | ENSG00000100324 | NP.705717 |
| chr3.0181428462 | −1.89 | 0.001 | 0.556 | ENSG00000242808 | NP.001177162 |
| chr5.0141852077 | −1.16 | 0.002 | 0.630 | ENSG00000231185 | |
| chr17.0014203257 | −2.60 | 0.002 | −0.82 | ENSG00000125430 | NP.006032 |
| chr18.0077960570 | 1.40 | 0.002 | −0.30 | K7ELH1 | |
| chr12.0031738230 | 1.02 | 0.002 | 0.638 | ENSG00000170456 | XP.005253385 |
| chr3.0063987349 | −0.68 | 0.002 | 0.775 | ENSG00000163635 | XP.005265422 |
| chr10.0056713225 | 0.761 | 0.002 | 0.693 | E7EM53 | |
| chr16.0032675959 | −1.52 | 0.004 | 0.581 | ENSG00000260311 | XP.005255564 |
| chr15.0089922792 | 1.37 | 0.004 | −0.27 | ENSG00000255571 | |
| chr2.0000729785 | −1.76 | 0.005 | 0.027 | ENSG00000227713 | — |
| chr14.0087905699 | −0.96 | 0.005 | 0.704 | ENSG00000258859 | — |
| chr20.0000642737 | 1.18 | 0.005 | −0.14 | S4R3F8 | — |
| chr1.0094703313 | −0.64 | 0.006 | 0.844 | ENSG00000137962 | — |
| chr2.0038724184 | −0.94 | 0.006 | 0.652 | ENSG00000231367 | — |
| chr8.0075917053 | 1.39 | 0.007 | 0.425 | ENSG00000121005 | NP.001273706 |
| chr14.0068286569 | −2.95 | 0.007 | 0.299 | A0A0A0MS18 | |
| chr17.0075087755 | −1.26 | 0.007 | 0.102 | ENSG00000234912 | — |
| chr15.0059785306 | 1.66 | 0.008 | 0.457 | A0A0U1RVI4 | — |
| chr6.0003738075 | −0.77 | 0.008 | 0.665 | PXDC1 | PX domain |
| | | | | | containing 1 |
| chr10.0104182130 | −1.25 | 0.009 | 0.639 | FBXL15 | F-box and leucine |
| | | | | | rich repeat |
| chr7.0097857306 | 0.614 | 0.013 | 0.718 | ENSG00000205356 | XP.005250311 |
| chr7.0150732834 | −0.61 | 0.013 | 0.728 | ENSG00000197150 | |
| chr4.0057366782 | −1.19 | 0.015 | 0.582 | D6RDY6 | — |
| chr15.0082762246 | −1.11 | 0.015 | 0.591 | ENSG00000188384 | NP.001157937 |
| chr16.0015680579 | −0.66 | 0.015 | 0.704 | ENSG00000166780 | |
| chr16.0002345736 | −0.56 | 0.018 | 0.761 | ENSG00000167972 | — |
| chr19.0010597219 | −0.64 | 0.019 | 0.717 | K7EJ49 | — |
| chr3.0146250349 | −0.69 | 0.019 | 0.727 | ENSG00000188313 | — |
| chr12.0021958070 | 1.21 | 0.020 | 0.527 | ENSG00000069431 | — |
| chr14.0023276073 | 1.07 | 0.020 | 0.526 | G3V5A1 | — |
| chr22.0021922517 | 1.64 | 0.021 | 0.434 | ENSG00000185651 | NP.001243285 |
| chr15.0075401874 | 1.68 | 0.024 | −0.71 | H3BU63 | — |

logFC = log[2] of fold change;
Response = up or down in Grp2 relative to Grpl.

Rankings of Sites for the Control Vs. DCIS Analysis

TABLE 9
Top 40 CpG Sites.
Rank ordered listing of CpG sites contributing the
most to the ordinate discrimination in the NMDS analysis.

chr14.0068286569
chr11.0057407074
chr22.0042678985
chr5.0141488834
chr18.0076481748
chr3.0181428462
chr15.0020733814
chr8.0086728497
chr2.0000729785
chr22.0021922517
chr15.0034635088
chr15.0059785306
chr20.0037899756
chr11.0093277585
chr17.0027912415
chr16.0032675959
chr22.0037428734
chr12.0069036581
chr11.0027384364
chr8.0075917053
chr2.0113240609
chr3.0057552599
chr16.0030366829
chr6.0163837639
chr11.0064514192
chr11.0075111078
chr14.0073330009
chr2.0220665390
chr17.0014666952
chr12.0067782441
chr12.0127210936
chr12.0057856280
chr11.0079339302
chr6.0008613853
chr12.0053719703
chr13.0028056134
chr20.0023474877
chr2.0204975304
chr7.0136556018
chr19.0016197423
chr17.0072921703
chr11.0065774760
chr16.0033297106
chr14.0023057582
chr5.0140782367
chr11.0063331532
chr8.0086556290
chr14.0019893202
chr2.0110873784
chr18.0040499812
chr9.0035603644
chr15.0102480783
chr9.0101559153
chr3.0184302159
chr16.0000766221
chr21.0015131488
chr3.0032549768
chr2.0096810269
chr16.0001393584
chr4.0057366782
chr2.0070315802
chr17.0080660575
chr6.0010695540
chr16.0012667415
chr5.0001211402
chr11.0067173421
chr15.0065823539
chr17.0036286187
chr12.0114476980
chr12.0021958070
chr6.0027794496
chr20.0004223373
chr21.0038068930
chr16.0089544877
chr3.0038081164
chr13.0028550394
chr3.0197382843
chr20.0040247763
chr11.0106894778
chr17.0050236261
chr11.0134525508
chr16.0049891504
chr15.0023931943
chr2.0177053201
chr16.0021512858
chr3.0039425054
chr3.0165780428
chr6.0080411829
chr15.0085872107
chr14.0090798926
chr8.0001978959
chr14.0055903656
chr17.0075087755
chr13.0114515221
chr2.0103775611
chrX.0118820440
chr17.0036280378
chr5.0174027285
chr2.0095966379
chr16.0089831979
chr17.0076136964
chr2.0074056574
chr19.0045458249
chr17.0014203257
chr6.0026157100
chr12.0094361472
chr15.0047477411
chr2.0233370968
chr12.0089918328
chr20.0031034124
chr11.0075989290
chr12.0065672210
chr11.0067045324
chr16.0089516632
chr11.0009853771
chr6.0036089235
chr5.0141852077
chr11.0009635545
chr22.0044280192
chr11.0134152806
chr15.0059157852
chr13.0029647211
chr17.0043176656
chr3.0062365451
chr18.0077960570
chr12.0006641855
chr16.0089554400
chr15.0082762246
chr15.0090456975
chr2.0007837366
chr14.0080887882
chr5.0142574738
chr2.0220384873
chr22.0019584506
chr22.0018371747
chr3.0180319542
chr2.0065141315
chr14.0100005173
chr20.0047082324
chr18.0010122170
chr22.0049764110
chr14.0037054111
chrX.0152973993
chr16.0005006250
chr12.0119054118
chr11.0036237395
chr2.0006503608
chr9.0133309320
chr18.0056589607
chr5.0077591726
chr5.0177002918
chr2.0073518897
chr3.0039891004
chr18.0001303942
chr13.0053530320
chr14.0023276073
chr6.0034192386
chr14.0061115424
chr8.0145743983
chr22.0021615439
chr22.0048020255
chr11.0069323971
chr2.0220340677
chr20.0062778083
chr11.0111460964
chr18.0035433091
chr16.0079802096
chr14.0024799383
chr19.0004882313
chr20.0035243876
chr2.0202126440
chr20.0041844969
chr12.0049958026
chr22.0044728672
chr13.0051569584
chr12.0075785138
chr15.0089922792
chr15.0098961407
chr3.0011212205
chr6.0028864916
chr2.0027775928
chr11.0049705897
chr6.0108486845
chr19.0004184748
chr17.0012693967
chr12.0031738230
chr16.0089894315
chr3.0126214661
chr19.0036037634
chr22.0039813308
chr11.0125249614
chr5.0151151794
chr11.0001913079
chr15.0092935856
chr6.0160220177
chr11.0130701567
chr13.0101091883
chr14.0020799205
chr11.0133835251
chr14.0097378133
chr3.0182881438
chr14.0037410282
chr15.0042652379
chr6.0159572466
chr12.0045271067
chr16.0000766588
chr6.0021597123
chr16.0086514423
chr6.0168665883
chr15.0065702632
chr9.0037578967
chr3.0195837439
chr3.0172166209
chr14.0074967635
chr3.0185619255
chr16.0089510331
chr4.0190991714
chr6.0136481416
chr14.0102929042
chr20.0060296084
chr17.0043663767
chr3.0108512761
chr17.0034491053
chr12.0032908207
chr12.0030704058
chr9.0099540553
chr2.0216947053
chr10.0003514879
chr18.0067634462
chr2.0086677764
chr14.0087905699
chr20.0058856822
chr3.0095072859
chr17.0070270313
chr13.0111107585
chr2.0241905384
chr14.0095008319
chr5.0141140612
chr15.0075495213
chr2.0175595489
chr17.0030556864
chr13.0022709340
chr8.0121857592
chr3.0071354612
chr15.0040212792
chr2.0000875249
chr18.0024586780
chr14.0084289630
chr14.0058593798
chr20.0000642737
chr16.0082712158
chr11.0038893798
chr5.0141048865
chr16.0011006670
chr20.0052164352
chr2.0229393549
chr3.0013924461
chr20.0055721756
chr5.0126114228
chr12.0133186738
chr22.0044729868
chr14.0099707257
chr12.0123424716
chr11.0044525297
chr11.0045280326
chr11.0126226385
chr11.0129510558
chr6.0149324892
chr3.0103361507
chr18.0068157667
chr22.0032044166
chr6.0079793340
chr5.0157965751
chr20.0056431925
chr14.0070327521
chr12.0120827122
chr2.0086609477
chr9.0102586890
chr22.0018834639
chr12.0014107327
chr12.0066279288
chr13.0065785631
chr8.0015398239
chr2.0241640727
chr12.0054379122
chr11.0118033212
chr7.0157964668
chr13.0048391192
chr20.0036799918
chr13.0111766619
chr3.0082619041
chr16.0006822158
chr5.0099954918
chr16.0011875269
chr20.0020837304
chr6.0163614535
chr12.0042190273
chr15.0075401874
chr11.0103295615
chr19.0046993480
chr12.0130155427
chr2.0042185584
chr18.0040051779
chr16.0089541352
chr11.0061451702
chr13.0028549550
chr17.0016916346
chr16.0002205666
chr18.0038227187
chr6.0031829093
chr2.0025018934
chr3.0181438281
chr13.0066518415
chr6.0139349539
chr12.0067254364
chr3.0013899697
chr22.0039627689
chr11.0119674018
chr14.0054457460
chr6.0035467808
chr17.0026810215
chr15.0045423374
chr3.0061788180
chr11.0001949013
chr22.0025490554
chr3.0075704892
chr3.0016536787
chr18.0024553398
chr5.0025749975
chr14.0023652825
chr2.0124783828
chr16.0000774754
chr11.0083236802
chr20.0033683440
chr16.0084043373
chr5.0061944346
chr12.0115112086
chr17.0070370553
chr18.0029523502
chr2.0038724184
chr20.0057075821
chr20.0031330621
chr22.0021612968
chr21.0037382085
chr6.0000401429
chr12.0093494772
chr13.0088862433
chr18.0046477561
chr20.0002827499
chr5.0141937225
chr13.0040770336
chr3.0171322486
chr17.0080193825
chr5.0091190508
chr15.0045428732
chr17.0018996904
chr18.0046303523
chr2.0118111021
chr5.0169624611
chr2.0058468651

The CpG list has been filtered for functional pathway assignments.
CpGs in hypothetical genes or unknown gene domains with unannotated functions were not included in this hard-copy listing, but all CpGs and their scores are retained in the database and are available for further analyses.

TABLE 10
Top 40 Hypermethylated Genes in Grpl.
Genes are presented with PFAM and RefSeq name designations as well as KEGG pathway associations.

| MethyLoad | UniProt | HUGO | Reactome |
|---|---|---|---|
| 3421.6 | C9JJ H3 | unk | Ub-specific.processing.proteases |
| 3025.2 | Q8NGC1 | OR11G2 | Olfactory.Signaling.Pathway |
| 2637.2 | Q8NGA5 | OR10H4 | Olfactory.Signaling.Pathway |
| 2619.1 | Q8NH85 | OR5R1 | Olfactory.Signaling.Pathway |
| 2601.9 | P10412 | HIST1H1E | Formation.of.Senescence-Associated.Heterochromatin.Foci. |
| 2436.4 | 095222 | OR6A2 | Olfactory.Signaling.Pathway |
| 2131.5 | Q96RD0 | OR8B2 | Olfactory.Signaling.Pathway |
| 2131.5 | Q8NHB1 | OR2V1 | Olfactory.Signaling.Pathway |
| 1883.5 | Q8NGK5 | OR52M1 | Olfactory.Signaling.Pathway |
| 1529.8 | Q8NGM9 | OR8D4 | Olfactory.Signaling.Pathway |
| 1352.6 | Q8NGP3 | OR5M9 | Olfactory.Signaling.Pathway |
| 1306.6 | P01563 | IFNA2 | TRAF6.mediated.IRF7.activation |
| 1305.8 | Q969M2 | GJA10 | Gap.junction.assembly |
| 1276.3 | P11021 | HSPA5 | Antigen.Presentation:.Folding,.assembly.and.peptide.load |
| 1240.9 | Q7L3B6 | CDC37L1 | Platelet.degranulation. |
| 1169.8 | Q8NGR5 | OR1L4 | Olfactory.Signaling.Pathway |
| 1130.5 | Q15615 | OR4D1 | Olfactory.Signaling.Pathway |
| 1125.8 | Q8NGS6 | OR13C3 | Olfactory.Signaling.Pathway |
| 1101.7 | Q8NH73 | OR4S2 | Olfactory.Signaling.Pathway |
| 1097.5 | 095006 | OR2F2 | Olfactory.Signaling.Pathway |
| 1061.5 | Q8NGY2 | OR6K2 | Olfactory.Signaling.Pathway |
| 1054.6 | Q8NH18 | OR5J2 | Olfactory.Signaling.Pathway |
| 1004.5 | Q3LFD5 | USP41 | ISG15.antiviral.mechanism |
| 990.4 | Q8NGD1 | OR4N2 | Olfactory.Signaling.Pathway |
| 976.1 | Q8NG41 | NPB | Peptide.ligand-binding.receptors |
| 968.0 | Q8NGT7 | OR2Al2 | Olfactory.Signaling.Pathway |
| 945.5 | Q15617 | OR8G1 | Olfactory.Signaling.Pathway |
| 928.1 | P59535 | TAS2R40 | G.alpha.(i).signalling.events |
| 897.1 | Q8NGA6 | OR1OH5 | Olfactory.Signaling.Pathway |
| 890.1 | P02533 | KRT14 | Type. 1.hemidesmosome.assembly |
| 888.9 | Q8NGVO | OR2Y1 | Olfactory.Signaling.Pathway |
| 870.4 | Q13145 | BAMBI | Downregulation.of.TGF-beta.receptor.signaling |
| 860.5 | P01567 | IFNA7 | TRAF6.mediated.IRF7.activation |
| 846.2 | Q8NGX0 | OR11L1 | Olfactory.Signaling.Pathway |
| 837.6 | Q5H8A3 | NMS | G.alpha.(q).signalling.events |
| 837.3 | P14652 | HOXB2 | Activation.of.anterior.HOX.genes.in.hindbrain.developmen |
| 833.9 | 002539 | HIST1H1A | Formation.of.Senescence-Associated.Heterochromatin.Foci. |
| 800.7 | Q99626 | CDX2 | Synthesis,.secretion,.and.inactivation.of.Glucagon-like. |
| 786.3 | Q86XQ3 | CATSPER3 | Sperm.Motility.And.Taxes |
| 785.4 | Q969N4 | TAAR8 | G.alpha.(s).signalling.events |

The gene list has been filtered for functional pathway assignments.
Hypothetical genes or genes with unannotated functions were not included in this hard-copy listing, but all genes and their scores are retained in the database and are available for further analyses.

TABLE 11
Top 40 Hypermethylated Genes in Grp2.
Genes are presented with PFAM and RefSeq name designations as well as KEGG pathway associations.

| MethyLoad | UniProt | HUGO | Reactome |
|---|---|---|---|
| −3592.3 | Q96RI9 | TAAR9 | G.alpha.(s).signalling.events |
| −2957.8 | Q8NGS3 | OR1J1 | Olfactory.Signaling.Pathway |
| −2601.6 | 060404 | OR10H3 | Olfactory.Signaling.Pathway |
| −2327.9 | Q8NGE9 | OR9Q2 | Olfactory.Signaling.Pathway |
| −2279.6 | Q9H1M4 | DEFB127 | Defensins |
| −2024.2 | Q9NV35 | NUDT15 | Phosphate.bond.hydrolysis.by.NUDT.proteins |
| −1869.5 | Q9NZPO | OR6C3 | Olfactory.Signaling.Pathway |
| −1864.1 | Q6IF42 | OR2A2 | Olfactory.Signaling.Pathway |
| −1795.5 | Q96L21 | RPL1OL | Nonsense.Mediated.Decay.(NMD).independent.of.the.Exon.Ju |
| −1748.8 | Q58HT5 | AWAT1 | Wax.biosynthesis |
| −1734.5 | Q9H2C8 | OR51V1 | Olfactory.Signaling.Pathway |
| −1594.0 | A6NL26 | OR5B21 | Olfactory.Signaling.Pathway |
| −1523.5 | Q8IXM3 | MRPL41 | Mitochondrial.tmnslation.initiation |
| −1491.8 | P09681 | GIP | G.alpha.(s).signalling.events |
| −1436.6 | Q6PGQ7 | BORA | Regulation.of.PLK1.Activity.at.G2/M.Transition |
| −1435.1 | Q8NH60 | OR52J3 | Olfactory.Signaling.Pathway. |
| −1415.5 | Q96PE6 | ZIM3 | Generic.Transcription.Pathway |
| −1378.4 | Q9H DDO | HRASLS | Acyl.chain.remodelling.of.PE |
| −1370.2 | P12104 | FABP2 | Hormone-sensitive.lipase.(HSL)-mediated.triacylglycerol. |
| −1355.3 | P00326 | ADH1C | Ethanol.oxidation |
| −1271.6 | P04118 | CLPS | Retinoid.metabolism.and.transport |
| −1267.8 | A6NP11 | ZNF716 | Generic.Transcription.Pathway |
| −1235.0 | Q96G91 | P2RY11 | G.alpha.(q).signalling.events |
| −1222.0 | Q8NGH7 | OR52L1 | Olfactory.Signaling.Pathway |
| −1218.0 | Q6VVB1 | NHLRC1 | Myoclonic.epilepsy.of.Lafora |
| −1162.0 | 076100 | OR7A10 | Olfactory.Signaling.Pathway |
| −1090.5 | Q8NGE5 | OR10A7 | Olfactory.Signaling.Pathway |
| −1078.8 | Q6ZYL4 | GTF2H5 | Dual.incision.in.TC-NER |
| −1073.1 | P62310 | LSM3 | mRNA.Splicing.-.Major.Pathway |
| −1053.5 | Q8NG99 | OR7G2 | Olfactory.Signaling.Pathway |
| −1003.7 | Q9BXT5 | TEX15 | Meiotic.recombination |
| −985.9 | Q6UXV4 | APOOL | Platelet.degranulation. |
| −981.8 | P22680 | CYP7A1 | Synthesis.of.bile.acids.and.bile.salts.via.27-hydroxycho |
| −966.7 | P30939 | HTR1F | G.alpha.(i).signalling.events |
| −954.9 | P81277 | PRLH | Peptide.ligand-binding.receptors |
| −950.8 | Q8NGS1 | OR1J4 | Olfactory.Signaling.Pathway |
| −950.5 | 030KP8 | DEFB136 | Defensins |
| −899.2 | Q9NYY3 | PLK2 | TP53.regulates.transcription.of.additional.cell.cycle.ge |
| −894.5 | Q6ZNIO | GCNT7 | O-linked.glycosylation.of.mucins |
| −867.5 | P61024 | CKS1B | Cyclin.D.associated.events.in.G1 |

The gene list has been filtered for functional pathway assignments.
Hypothetical genes or genes with unannotated functions were not included in this hard-copy listing, but all genes and their scores are retained in the database and are available for further analyses.

TABLE 12
Top 30 Hypermethylated Pathways in Grp1.
Pathways are rank ordered by methylation load score (ΔML) and
include a list of the top 10 scoring genes contributing to the
pathways load value. The gene list is organized
in decreasing numerical order of the individual gene ΔML scores.

| MethyLoad | Pathway | Genes |
|---|---|---|
| 759.0 | Rheumatoid.arthritis | Q16552 |
| 662.9 | B.cell.receptor.signaling.path | Q9UN19 |
| 660.5 | Tryptophan.metabolism | unk |
| 550.7 | Streptomycin.biosynthesis | 095455 |
| 518.8 | Arrhythmogenic.right.ventricul | P17302 |
| 410.6 | Basal.cell.carcinoma | P12643 |
| 397.3 | Pantothenate.CoA.biosynthesis | 095497 |
| 372.9 | Autoimmune.thyroid.disease | A0A0R4J2F0 |
| 363.9 | Metabolism.xenobiotics.by.cyto | 095154 |
| 360.3 | Chloroalkane.chloroalkene.degr | P30837 |
| 315.9 | Neurotrophin.signaling.pathway | unk |
| 287.3 | Aldosteroneregulated.sodium.re | unk |
| 271.9 | Bisphenol.degradation | unk |
| 268.9 | Phagosome | Q5KU26 |
| 267.1 | Proximal.tubule.bicarbonate.re | P49448 |
| 262.8 | Intestinal.immune.network.for. | P01833 |
| 234.6 | Tight.junction | Q15334, P51153, |
| | | P20337 |
| 221.3 | Fatty.acid.elongation | Q9GZR5, Q9HB03 |
| 218.0 | Amyotrophic.lateral.sclerosis. | E7ESP9, P04040, |
| | | P12036 |
| 216.7 | Reninangiotensin.system | unk |
| 202.1 | Circadian.entrainment | P48039, B7ZA25 |
| 199.4 | Glutathione.metabolism | Q96SL4 |
| 197.5 | Vasopressinregulated.water.rea | unk |
| 194.8 | Leishmaniasis | P01583, P12314, |
| | | P49006, P23458 |
| 189.4 | Collecting.duct.acid.secretion | P51164, P02730 |
| 178.6 | Melanogenesis | P14679, 001726 |
| 175.3 | Chemokine.signaling.pathway | P10720, Q9Y4X3, |
| | | O00626, P22362, |
| | | O43927, P47992, |
| | | P02776, P80075, |
| 174.8 | Pancreatic.secretion | P32238 |
| 169.6 | Phenylpropanoid.biosynthesis | P30041 |
| 167.9 | Novobiocin biosynthesis | P17735 |

Gene order within each pathway is determined by decreasing methylation load scores (ΔML).
The first gene in the list contributes the most to the pathway methylation score.

TABLE 13
Top 30 Hypermethylated Pathways in Grp2. Pathways are rank ordered by
methylation load score (Δ M L) and include a list of the top 10
scoring genes contributing to the pathways load value. The gene list
is organized in decreasing numerical order of the individual gene Δ M L scores.

| MethyLoad | Pathway | Genes |
|---|---|---|
| −619.0 | Malaria | P07996, Q12918 |
| −574.4 | Cell.cycle.yeast | unk |
| −546.0 | Meiosis.yeast | 014566 |
| −518.3 | Inositol.phosphate.metabolism | Q8NFU5 |
| −470.1 | Twocomponent.system | unk |
| −458.9 | Synaptic.vesicle.cycle | Q7Z7G2 |
| −444.7 | Taurine.hypotaurine.metabolism | 016878, Q96SZ5 |
| −442.6 | Antigen.processing.presentatio | unk |
| −432.0 | JakSTAT.signaling.pathway | unk |
| −392.5 | Atrazine.degradation | unk |
| −370.3 | Thyroid.cancer | 016204 |
| −353.1 | Nonhomologous.endjoining | unk |
| −350.9 | Valine.leucine.isoleucine.degr | unk |
| −338.4 | Toluene.degradation | Q96DG6 |
| −333.1 | Apoptosis | Q14249 |
| −326.6 | Lipoic.acid.metabolism | unk |
| −291.4 | Natural.killer.cell.mediated.c | Q9BZM5, Q9BZM6 |
| −289.4 | Base.excision.repair | unk |
| −278.8 | Phototransduction.fly | unk |
| −277.8 | Glycosphingolipid.biosynthesis | 043505, P19526, Q9Y231, Q8NDV1 |
| −269.7 | Glycosylphosphatidylinositolan | unk |
| −214.7 | Viral.carcinogenesis | A0A0A0MSJ9, 014839, P51679 |
| −212.2 | African.trypanosomiasis | Q6ZQW0, P55085, P22301 |
| −204.2 | Insulin.secretion | P09681, P43220, Q8TDV5, 014842 |
| −196.2 | Peroxisome | Q567V2, Q9BY49, P47989, P56589, P21549, |
| | | 095822, Q9UBJ2, A8MXV4, Q15126, Q9UJM8 |
| −182.5 | Mismatch.repair | unk |
| −182.1 | Sulfur.relay.system | Q7Z7A3, 095396 |
| −181.6 | Methane.metabolism | P05062 |
| −177.6 | Terpenoid.backbone.biosynthesi | 075844, Q9BXS1 |
| −170.4 | Shigellosis | P60673 |

Gene order within each pathway is determined by decreasing methylation load scores (Δ M L). The first gene in the list contributes the most to the pathway methylation score.

TABLE 14
Top 40 CpG Sites by P-value. Rank ordered listing of CpG sites in known UniProt genes are sorted by P-values
adjusted for false discovery rate (FDR) threshold. Site-specific dispersion was estimated to equalize
CpG variances. A Likelihood Ratio Test was used with a defined one-way ANOVA model for pairwise tests.

| CpG Site | logFC | FDR adj-P-val | Response | Gene | Description |
|---|---|---|---|---|---|
| chr14.0068286569 | −2.95 | 5.51e−20 | down | A0A0A0MS18 | — |
| chr11.0057407074 | 2.06 | 7.29e−13 | up | ENSG00000254602 | — |
| chr22.0042678985 | −2.12 | 2.20e−11 | down | ENSG00000100207 | — |
| chr5.0141488834 | −1.96 | 1.19e−09 | down | NDFIP1 | Nedd4 family interacting prote |
| chr3.0181428462 | −1.89 | 4.54e−09 | down | ENSG00000242808 | NP.001177162 |
| chr2.0000729785 | −1.76 | 6.93e−08 | down | ENSG00000227713 | — |
| chr22.0021922517 | 1.64 | 8.49e−08 | up | ENSG00000185651 | NP.001243285 |
| chr15.0034635088 | 1.58 | 6.02e−07 | up | H0YM60 | — |
| chr15.0059785306 | 1.66 | 6.33e−07 | up | A0A0U1RVI4 | — |
| chr20.0037899756 | 1.58 | 6.43e−07 | up | ENSG00000211534 | — |
| chr17.0027912415 | −1.56 | 7.48e−06 | down | ENSG00000264031 | NP.937790 |
| chr16.0032675959 | −1.52 | 9.69e−06 | down | ENSG00000260311 | XP.005255564 |
| chr12.0069036581 | 1.69 | 2.66e−05 | up | F5H7Y6 | — |
| chr11.0027384364 | 2.29 | 5.44e−05 | up | ENSG00000109881 | NP.542385 |
| chr8.0075917053 | 1.39 | 7.34e−05 | up | ENSG00000121005 | NP.001273706 |
| chr2.0113240609 | −2.18 | 7.34e−05 | down | TTL | tubulin tyrosine ligase |
| chr16.0030366829 | 1.85 | 0.000 | up | ENSG00000260219 | NP.001230575 |
| chr6.0163837639 | 1.73 | 0.000 | up | H0YGD6 | — |
| chr11.0064514192 | 1.34 | 0.000 | up | ENSG00000068976 | NP.005600 |
| chr11.0075111078 | 2.19 | 0.000 | up | H0YF32 | — |
| chr14.0073330009 | 1.33 | 0.000 | up | ENSG00000205683 | NP.036206 |
| chr12.0127210936 | 1.47 | 0.000 | up | ENSG00000189238 | NP.001273148 |
| chr12.0057856280 | −2.05 | 0.000 | down | ENSG00000111087 | NP.001153517 |
| chr12.0053719703 | 2.22 | 0.000 | up | F8VV67 | — |
| chr20.0023474877 | 1.29 | 0.001 | up | CST8 | cystatin 8 |
| chr7.0136556018 | 1.25 | 0.001 | up | ENSG00000234352 | — |
| chr14.0023057582 | −3.16 | 0.001 | down | F5GXX5 | — |
| chr19.0016197423 | −1.39 | 0.001 | down | ENSG00000167460 | — |
| chr17.0072921703 | −1.43 | 0.001 | down | ENSG00000183034 | NP.835454 |
| chr16.0033297106 | −1.30 | 0.001 | down | ENSG00000262090 | XP.005255564 |
| chr8.0086556290 | −8.37 | 0.001 | down | ENSG00000270971 | — |
| chr5.0140782367 | −1.94 | 0.001 | down | ENSG00000204956 | NP.114382 |
| chr14.0019893202 | 2.02 | 0.001 | up | ENSG00000244306 | NP.001005356 |
| chr2.0110873784 | −1.26 | 0.001 | down | F8WE57 | — |
| chr18.0040499812 | −1.28 | 0.001 | down | ENSG00000152214 | — |
| chr9.0035603644 | 2.03 | 0.001 | up | ENSG00000107140 | NP.006276 |
| chr9.0101559153 | −1.26 | 0.002 | down | ENSG00000165138 | — |
| chr16.0000766221 | 1.45 | 0.002 | up | H3BUM1 | — |
| chr3.0184302159 | 1.57 | 0.002 | up | H7C2X0- | |
| chr3.0032549768 | 1.69 | 0.002 | up | ENSG00000213849 | NP.060271 |

logFC = log[2] of fold change; Response = up or down in Grp2 relative to Grp1.

Rankings of Sites for the Control Vs. Invasive Breast Cancer Analysis

TABLE 15
Top 40 CpG Sites. Rank ordered listing of CpG sites contributing
the most to the ordinate discrimination in the NMDS analysis.

chr2.0113240609
chr11.0031846870
chr12.0050297405
chr17.0073268308
chr9.0037578967
chr19.0042069923
chr3.0042911177
chr4.0094751025
chr11.0007695679
chr14.0056407135
chr2.0086609477
chr21.0045561163
chr14.0065689243
chr3.0033482718
chr15.0074630994
chr17.0014203257
chr17.0042734551
chr10.0003514879
chr15.0035449759
chr11.0017553236
chr11.0134341441
chr3.0105652482
chr14.0023057582
chr2.0183731397
chr20.0000565588
chr12.0118518389
chr11.0043580678
chr15.0057967982
chr13.0088862433
chr3.0071081288
chr2.0008833634
chr11.0083251309
chr10.0056713225
chr13.0112720793
chr17.0077216743
chr3.0070322314
chr20.0002584692
chr11.0125011371
chr15.0020733814
chr3.0155945555
chr22.0042564578
chr19.0000925223
chr2.0101706746
chr20.0049747671
chr15.0075362233
chr9.0127119764
chr15.0093634307
chr3.0036074151
chr6.0085777767
chr11.0093707469
chr12.0074415833
chr12.0005258607
chr6.0052456152
chr12.0002930852
chr20.0014745674
chr14.0091072652
chr16.0085461847
chr13.0076387384
chr3.0028035684
chr18.0008659584
chr5.0015758651
chr12.0050506409
chr18.0044774814
chr20.0009496892
chr3.0138656356
chr16.0055539404
chr11.0068152468
chr5.0003103410
chr13.0068862091
chr20.0046305398
chr3.0125872130
chr17.0050236261
chr11.0036057669
chr11.0074125328
chr6.0073330966
chr12.0113313505
chr19.0033752620
chr17.0017654366
chr8.0080740825
chr6.0079793340
chr16.0088963738
chr16.0028935921
chr13.0093953330
chr22.0049828000
chr5.0176889569
chr15.0043975270
chr2.0000729785
chr15.0090638902
chr17.0021023063
chr8.0142485152
chr3.0132377906
chr6.0079911924
chr3.0018465177
chr22.0023248614
chr5.0076928892
chr5.0159604108
chr2.0073152645
chr14.0076953170
chr13.0113636156
chr6.0038683255
chr12.0132671272
chr20.0055721756
chr2.0155639299
chr16.0023763882
chr16.0027257120
chr17.0036280378
chr15.0066462172
chr11.0075051113
chr11.0070512616
chr2.0220665390
chr15.0058173088
chr2.0125572919
chr2.0020336517
chr13.0100075263
chr10.0116061880
chr6.0021807995
chr14.0074967635
chr7.0102182417
chr11.0055979234
chr22.0030973469
chr20.0042136963
chr20.0062486416
chr2.0071651284
chr3.0000065214
chr20.0002827499
chr11.0009636058
chr7.0121048611
chr16.0049888100
chr17.0007239160
chr5.0038246603
chr22.0044858743
chr3.0054161016
chr13.0032377061
chr11.0127547963
chr20.0023017832
chr3.0009113874
chr17.0007465780
chr2.0003743921
chr3.0064018099
chr15.0101300081
chr17.0035447333
chr11.0044340428
chr12.0096632313
chr3.0015674946
chr17.0078942509
chr18.0019747084
chr5.0121414025
chr9.0129386447
chr22.0040316505
chr13.0022763192
chr19.0031744952
chr12.0047401732
chr14.0101144066
chr13.0100648209
chr14.0061834869
chr11.0111101254
chr11.0066816866
chr3.0126259929
chr9.0140657192
chr20.0061554910
chr22.0018811858
chr17.0041322124
chr20.0010982737
chr20.0057227608
chr20.0033683440
chr11.0134746717
chr7.0097857306
chr6.0134210997
chr19.0045073719
chr12.0031738230
chr5.0007414317
chr15.0042357486
chr2.0045029060
chr3.0134125941
chr4.0009215375
chr20.0020742515
chr19.0039889239
chr19.0034397576
chr17.0079402697
chr16.0065259721
chr2.0059252357
chr20.0044171320
chr12.0131694264
chr17.0072174025
chr12.0013319824
chr20.0031261476
chr15.0058158361
chr14.0035026525
chr2.0011767102
chr13.0082545364
chr5.0141309248
chr11.0076370946
chr20.0061045252
chr14.0106416958
chr17.0066196740
chr19.0041248943
chr12.0106631508
chr18.0076481748
chr2.0170276122
chr3.0111632475
chr6.0003563820
chr18.0011284750
chr14.0070497701
chr6.0112535805
chr8.0075917053
chr11.0001227691
chr5.0068950088
chr11.0046564148
chr15.0075983228
chr18.0027861099
chr15.0048151460
chr2.0068179208
chr12.0067143488
chr17.0062294803
chr12.0052961748
chr11.0001945540
chr15.0069195045
chr16.0052458588
chr22.0032583409
chr15.0071076473
chr22.0022296520
chr9.0116344785
chrX.0125349552
chr14.0035099669
chr17.0045858885
chr13.0027455420
chr9.0090256959
chr16.0086609680
chr20.0060909671
chr17.0016267297
chr14.0102929042
chr11.0120384679
chr10.0060305797
chr9.0122756999
chr2.0016081660
chr11.0134147815
chr11.0014384377
chr12.0089768127
chr11.0124669378
chr14.0097592491
chr20.0062778083
chr12.0021958070
chr5.0148574900
chr20.0055927974
chr1.0181104403
chr18.0052876612
chr11.0045202699
chr12.0095162637
chr9.0100109657
chr16.0057337873
chr2.0029419721
chr19.0011750942
chr20.0023032166
chr11.0044067218
chr13.0033591525
chr7.0101939610
chr2.0236404318
chr11.0091598917
chr10.0119493401
chr15.0059785306
chr14.0093527696
chr11.0009853771
chr2.0039665281
chr22.0050455644
chr12.0072332759
chr12.0129282241
chr7.0098979512
chr13.0022450119
chr17.0001954986
The CpG list has been filtered for functional pathway assignments. CpGs in hypothetical genes or unknown gene domains with unannotated functions were not included in this hard-copy listing, but all CpGs and their scores are retained in the database and are available for further analyses.

TABLE 16
Top 40 Hypermethylated Genes in Grp1. Genes are presented with PFAM
and RefSeq name designations as well as KEGG pathway associations.

| MethyLoad | UniProt | HUGO | Reactome |
|---|---|---|---|
| 3040.1 | Q9NV35 | NUDT15 | Phosphate.bond.hydrolysis.by.NUDT.proteins |
| 2505.3 | Q8NGC1 | OR11G2 | Olfactory.Signaling.Pathway |
| 2200.4 | Q8NH73 | OR4S2 | Olfactory.Signaling.Pathway |
| 2104.4 | C9JJH3 | unk | Ub-specific.processing.proteases |
| 2098.6 | Q8NGD5 | OR4K14 | Olfactory.Signaling.Pathway |
| 2097.5 | P10412 | HIST1H1E | Formation.of.Senescence-Associated.Heterochromatin.Foci. |
| 2067.1 | Q8NGR9 | OR1N2 | Olfactory.Signaling.Pathway |
| 1835.0 | Q9P032 | NDUFAF4 | Complex.I.biogenesis |
| 1821.3 | Q8NGP3 | OR5M9 | Olfactory.Signaling.Pathway |
| 1651.7 | P08620 | FGF4 | FGFR3c.ligand.binding.and.activation |
| 1592.7 | Q8NH18 | OR5J2 | Olfactory.Signaling.Pathway |
| 1477.7 | Q6IFN5 | OR7E24 | Olfactory.Signaling.Pathway |
| 1444.0 | Q969N4 | TAAR8 | G.alpha.(s).signalling.events |
| 1298.9 | Q96RD0 | OR8B2 | Olfactory.Signaling.Pathway |
| 1175.7 | Q8N146 | OR8H3 | Olfactory.Signaling.Pathway |
| 1162.2 | Q7L2Z9 | CENPQ | Deposition.of.new.CENPA-containing.nucleosomes.at.the.ce |
| 1130.3 | O95222 | OR6A2 | Olfactory.Signaling.Pathway |
| 1095.9 | Q9ULW2 | FZD10 | Class.B/2.(Secretin.family.receptors) |
| 1074.7 | Q7RTS3 | PTF1A | Regulation.of.gene.expression.in.early.pancreatic.precur |
| 1035.1 | Q8NHB1 | OR2V1 | Olfactory.Signaling.Pathway |
| 1026.0 | Q8NGA5 | OR10H4 | Olfactory.Signaling.Pathway |
| 1006.4 | Q96RI8 | TAAR6 | G.alpha.(s).signalling.events |
| 993.9 | Q8NGR5 | OR1L4 | Olfactory.Signaling.Pathway |
| 992.2 | Q53H54 | TRMT12 | Synthesis.of.wybutosine.at.G37.of.tRNA(Phe) |
| 987.2 | Q8NGE3 | OR10P1 | Olfactory.Signaling.Pathway |
| 984.1 | Q9H208 | OR10A2 | Olfactory.Signaling.Pathway |
| 982.3 | Q6P1L8 | MRPL14 | Mitochondrial.translation.initiation |
| 958.5 | Q8NGJ4 | OR52E2 | Olfactory.Signaling.Pathway |
| 916.6 | Q8NGH3 | OR2D3 | Olfactory.Signaling.Pathway |
| 910.4 | P47985 | UQCRFS1 | Respiratory.electron.transport |
| 897.3 | Q8NGW1 | OR6B3 | Olfactory.Signaling.Pathway |
| 886.0 | Q8NGK4 | OR52K1 | Olfactory.Signaling.Pathway |
| 877.4 | Q96RJ0 | TAAR1 | G.alpha.(s).signalling.events |
| 875.1 | P52961 | ART1 | Alpha-defensins |
| 867.3 | Q8NGS4 | OR13F1 | Olfactory.Signaling.Pathway |
| 860.2 | Q8NGY2 | OR6K2 | Olfactory.Signaling.Pathway |
| 852.4 | Q8NGT7 | OR2A12 | Olfactory.Signaling.Pathway |
| 849.3 | D6R901 | unk | Ub-specific.processing.proteases |
| 817.1 | Q14973 | SLC10A1 | Recycling.of.bile.acids.and.salts |
| 813.0 | Q9UBS3 | DNAJB9 | XBP1(S).activates.chaperone.genes |

The gene list has been filtered for functional pathway assignments. Hypothetical genes or genes with unannotated functions were not included in this hard-copy listing, but all genes and their scores are retained in the database and are available for further analyses.

TABLE 17
Top 40 Hypermethylated Genes in Grp2. Genes are presented with PFAM
and RefSeq name designations as well as KEGG pathway associations.

| MethyLoad | UniProt | HUGO | Reactome |
|---|---|---|---|
| −2613.2 | Q8NGH7 | OR52L1 | Olfactory.Signaling.Pathway |
| −2458.3 | Q8NGE9 | OR9Q2 | Olfactory.Signaling.Pathway |
| −2314.7 | Q58HT5 | AWAT1 | Wax.biosynthesis |
| −2272.4 | Q8NH69 | OR5W2 | Olfactory.Signaling.Pathway |
| −2132.1 | Q8NGT0 | OR13C9 | Olfactory.Signaling.Pathway |
| −2014.5 | Q96RI9 | TAAR9 | G.alpha.(s).signalling.events |
| −1869.9 | Q96RA2 | OR7D2 | Olfactory.Signaling.Pathway |
| −1843.8 | Q12918 | KLRB1 | Immunoregulatory.interactions.between.a.Lymphoid.and.a.n |
| −1809.7 | Q30KQ1 | DEFB133 | Defensins |
| −1784.1 | Q96PL1 | SCGB3A2 | Scavenging.by.Class.A.Receptors |
| −1758.9 | Q9Y2Y1 | POLR3K | RNA.Polymerase.III.Chain.Elongation |
| −1664.5 | P82930 | MRPS34 | Mitochondrial.translation.initiation |
| −1519.6 | Q9HDD0 | HRASLS | Acyl.chain.remodelling.of.PE |
| −1515.1 | Q8IXM3 | MRPL41 | Mitochondrial.translation.initiation |
| −1355.5 | O76100 | OR7A10 | Olfactory.Signaling.Pathway |
| −1342.8 | Q9GZK3 | OR2B2 | Olfactory.Signaling.Pathway |
| −1331.8 | Q6IF42 | OR2A2 | Olfactory.Signaling.Pathway |
| −1323.8 | Q96L33 | RHOV | Rho.GTPase.cycle |
| −1306.8 | Q30KQ7 | DEFB113 | Defensins |
| −1267.0 | Q6ZYL4 | GTF2H5 | Dual.incision.in.TC-NER |
| −1263.4 | Q8NGS3 | OR1J1 | Olfactory.Signaling.Pathway |
| −1215.0 | Q6IF63 | OR52W1 | Olfactory.Signaling.Pathway |
| −1190.3 | Q8NGC3 | OR10G2 | Olfactory.Signaling.Pathway |
| −1178.5 | Q96L21 | RPL10L | Nonsense.Mediated.Decay.(NMD).independent.of.the.Exon.Ju |
| −1163.4 | Q8NGD4 | OR4K1 | Olfactory.Signaling.Pathway |
| −1155.0 | Q8NG99 | OR7G2 | Olfactory.Signaling.Pathway |
| −1138.9 | Q96A08 | HIST1H2BA | Recruitment.and.ATM-mediated.phosphorylation.of.repair.a |
| −1100.6 | Q8NGQ4 | OR10Q1 | Olfactory.Signaling.Pathway |
| −1082.4 | A6NHG9 | OR5H14 | Olfactory.Signaling.Pathway |
| −1074.7 | Q9GZQ4 | NMUR2 | G.alpha.(q).signalling.events |
| −1074.0 | Q9BYW3 | DEFB126 | Defensins |
| −1004.4 | Q9NUP1 | BLOC1S4 | Golgi.Associated.Vesicle.Biogenesis |
| −998.9 | P62310 | LSM3 | mRNA.Splicing.-.Major.Pathway |
| −987.6 | Q8NGP6 | OR5M8 | Olfactory.Signaling.Pathway |
| −974.2 | Q96FJ2 | DYNLL2 | Intraflagellar.transport |
| −928.1 | 095221 | OR5F1 | Olfactory.Signaling.Pathway |
| −926.7 | Q8NGE5 | OR10A7 | Olfactory.Signaling.Pathway |
| −914.4 | Q8WZ84 | OR8D1 | Olfactory.Signaling.Pathway |
| −885.5 | Q08ER8 | ZNF543 | Generic.Transcription.Pathway |
| −853.4 | 095813 | CER1 | Signaling.by.NODAL |

The gene list has been filtered for functional pathway assignments. Hypothetical genes or genes with unannotated functions were not included in this hard-copy listing, but all genes and their scores are retained in the database and are available for further analyses.

TABLE 18
Top 30 Hypermethylated Pathways in Grp1. Pathways are rank ordered by
methylation load score (Δ M L) and include a list of the top 10
scoring genes contributing to the pathways load value. The gene list
is organized in decreasing numerical order of the individual gene Δ M L scores.

| MethyLoad | Pathway | Genes |
|---|---|---|
| 497.5 | RNA.polymerase | Q3B726 |
| 471.4 | Fructose.mannose.metabolism | Q9NQ88 |
| 443.9 | Aldosteroneregulated.sodium.re | unk |
| 433.9 | Prion.diseases | P11021, P10643 |
| 420.0 | Tryptophan.metabolism | unk |
| 372.4 | Novobiocin.biosynthesis | P17735 |
| 320.6 | Prostate.cancer | Q12778 |
| 320.1 | Chloroalkane.chloroalkene.degr | P30837 |
| 319.0 | Homologous.recombination | O43543 |
| 299.6 | Leishmaniasis | P49006, P01583, P23458, P12314 |
| 297.6 | Primary.bile.acid.biosynthesis | O95992 |
| 291.2 | Neurotrophin.signaling.pathway | unk |
| 280.0 | Streptomycin.biosynthesis | O95455 |
| 258.2 | ABC.transporters | Q9NRK6 |
| 255.2 | Circadian.rhythm | O14503, P20393, Q9C0J9 |
| 244.9 | Adipocytokine.signaling.pathwa | P41159, Q86V24 |
| 236.5 | Biosynthesis.ansamycins | Q9H0I9 |
| 234.6 | Pantothenate.CoA.biosynthesis | O95497 |
| 214.3 | Lysine.degradation | A0A0C4DFR3, A0A0A0MQV9 |
| 213.4 | Basal.cell.carcinoma | P12643 |
| 213.3 | Lipoic.acid.metabolism | unk |
| 211.7 | Carbohydrate.digestion.absorpt | unk |
| 210.4 | Gap.junction | Q9UKL4 |
| 208.8 | Arrhythmogenic.right.ventricul | P17302 |
| 208.6 | Intestinal.immune.network.for. | P01833 |
| 207.5 | MAPK.signaling.pathway | Q05923, Q16690 |
| 204.8 | Circadian.entrainment | P48039, B7ZA25 |
| 198.0 | Progesteronemediated.oocyte.ma | unk |
| 174.5 | Amino.sugar.nucleotide.sugar.m | Q8TBE9 |
| 173.3 | Glutathione.metabolism | Q96SL4 |

Gene order within each pathway is determined by decreasing methylation load scores. The first gene in the list contributes the most to the pathway methylation score.

TABLE 19
Top 30 Hypermethylated Pathways in Grp2. Pathways are rank ordered by
methylation load score (Δ M L) and include a list of the top 10
scoring genes contributing to the pathways load value. The gene list
is organized in decreasing numerical order of the individual gene Δ M L scores.

| MethyLoad | Pathway | Genes |
|---|---|---|
| −764.8 | Starch.sucrose.metabolism | Q6PCE3 |
| −445.7 | Butanoate.metabolism | P0C7M7 |
| −427.1 | Antigen.processing.presentatio | unk |
| −423.1 | Malaria | QI2918, P07996 |
| −418.1 | Shigellosis | P60673 |
| −407.7 | Endocrine.other.factorregulate | unk |
| −399.6 | Taurine.hypotaurine.metabolism | Q16878, Q96SZ5 |
| −367.2 | Toluene.degradation | Q96DG6 |
| −366.8 | Glyeine.serine.threonine.metab | unk |
| −366.5 | Valine.leucine.isoleucine.degr | unk |
| −328.9 | Pancreatic.cancer | unk |
| −326.9 | Oocyte.meiosis | Q9UQE7 |
| −317.4 | Glycosphingolipid.biosynthesis | O43505, Q9Y231, P19526, Q8NDV1 |
| −314.6 | Osteoclast.differentiation | Q9HBY0 |
| −288.8 | Nonhomologous.endjoining | unk |
| −282.5 | Sulfur.relay.system | Q7Z7A3, O95396 |
| −281.2 | Other.glycan.degradation | Q9Y3R4, P04066 |
| −250.2 | Reninangiotensin.system | unk |
| −240.5 | Cell.cycle.yeast | unk |
| −226.2 | Thyroid.cancer | Q16204 |
| −223.6 | Complement.coagulation.cascade | P07204, P05160, P00748, P01008 |
| −216.9 | Inositol.phosphate.metabolism | Q8NFU5 |
| −214.6 | Small.cell.lung.cancer | P33552, P61024 |
| −198.3 | Calcium.signaling.pathway | unk |
| −194.6 | Glycerolipid.metabolism | Q17RR3, Q96AD5, O60218 |
| −194.0 | African.trypanosomiasis | Q6ZQW0, P55085, P22301 |
| −193.8 | Natural.killer.cell.mediated.c | Q9BZM5, Q9BZM6 |
| −189.9 | Protein.export | P61009 |
| −185.2 | Synaptic.vesicle.cycle | Q7Z7G2 |
| −183.8 | Glutamatergic.synapse | unk |

Gene order within each pathway is determined by decreasing methylation load scores (Δ M L). The first gene in the list contributes the most to the pathway methylation score.

TABLE 20
Top 40 CpG Sites by P-value. Rank ordered listing of CpG sites in known UniProt genes are sorted by P-values
adjusted for false discovery rate (FDR) threshold. Site-specific dispersion was estimated to equalize
CpG variances. A Likelihood Ratio Test was used with a defined one-way ANOVA model for pairwise tests.

| CpG Site | logFC | FDR adj pP-val | Response | Gene | Description |
|---|---|---|---|---|---|
| chr2.0113240609 | −2.18 | 2.59e−11 | down | TTL | tubulin tyrosine ligase |
| chr12.0050297405 | −1.30 | 2.27e−09 | down | F8VV65 | — |
| chr11.0031846870 | −1.75 | 3.55e−09 | down | H0YDA4 | — |
| chr17.0073268308 | −1.21 | 7.79e−07 | down | ENSG00000263843 | — |
| chr4.0094751025 | 0.991 | 4.85e−06 | up | ATOH1 | atonal bHLH transcription fact |
| chr9.0037578967 | 1.49 | 4.97e−06 | up | ENSG00000147912 | — |
| chr19.0042069923 | −1.39 | 4.97e−06 | down | ENSG00000007129 | XP.005259429 |
| chr11.0007695679 | −1.35 | 4.97e−06 | down | ENSG00000166394 | — |
| chr21.0045561163 | 1.05 | 1.20e−05 | up | H7C2G3 | — |
| chr15.0074630994 | 0.997 | 1.20e−05 | up | ENSG00000140459 | — |
| chr17.0042734551 | −1.42 | 2.27e−05 | down | ENSG00000180336 | — |
| chr17.0014203257 | −2.23 | 5.20e−05 | down | ENSG00000125430 | NP.006032 |
| chr12.0118518389 | 0.994 | 6.60e−05 | up | F5H724 | — |
| chr11.0134341441 | −1.33 | 6.89e−05 | down | ENSG00000255545 | NP.061114 |
| chr11.0017553236 | −1.28 | 8.55e−05 | down | E9PNW1 | — |
| chr2.0183731397 | 1.83 | 0.000 | up | FRZB | frizzled-related protein |
| chr3.0071081288 | −1.05 | 0.000 | down | ENSG00000114861 | NP.001012523 |
| chr10.0056713225 | 0.818 | 0.000 | up | E7EM53 | — |
| chr13.0088862433 | 1.23 | 0.000 | up | ENSG00000231019 | — |
| chr13.0112720793 | −2.18 | 0.000 | down | SOX1 | SRY-box 1 |
| chr11.0043580678 | −1.55 | 0.000 | down | ENSG00000149084 | — |
| chr20.0002584692 | 1.18 | 0.000 | up | TMC2 | transmembrane channel like 2 |
| chr3.0155945555 | −1.40 | 0.000 | down | H7C4S9 | — |
| chr3.0033482718 | −2.32 | 0.000 | down | C9JWL3 | — |
| chr20.0014745674 | 0.900 | 0.001 | up | ENSG00000172264 | — |
| chr14.0091072652 | 1.09 | 0.001 | up | ENSG00000165914 | — |
| chr13.0076387384 | 0.828 | 0.001 | up | F8WD26 | — |
| chr12.0002930852 | 1.31 | 0.001 | up | ENSG00000111203 | XP.005253766 |
| chr5.0015758651 | 0.966 | 0.001 | up | J3KNM9 | — |
| chr20.0046305398 | 0.777 | 0.001 | up | ENSG00000196562 | XP.005260516 |
| chr12.0050506409 | −1.57 | 0.001 | down | ENSG00000178449 | XP.005269256 |
| chr20.0049747671 | −1.00 | 0.001 | down | ENSG00000228820 | — |
| chr20.0009496892 | −1.49 | 0.002 | down | ENSG00000125869 | NP.036393 |
| chr6.0073330966 | −1.09 | 0.002 | down | A0A0A0MT07 | — |
| chr18.0044774814 | 2.04 | 0.002 | up | ENSG00000215474 | NP.001032891 |
| chr3.0138656356 | −1.87 | 0.002 | down | ENSG00000244578 | — |
| chr11.0036057669 | 1.18 | 0.002 | up | ENSG00000179241 | NP.777562 |
| chr19.0000925223 | −1.08 | 0.002 | down | K7EJ04 | — |
| chr2.0000729785 | −1.45 | 0.003 | down | ENSG00000227713 | — |
| chr17.0050236261 | 1.72 | 0.003 | up | ENSG00000154975 | — |

logFC = log[2] of fold change; Response = up or down in Grp2 relative to Grp1.

Rankings of Sites for the DCIS Vs. Invasive Analysis

TABLE 21
Top 40 CpG Sites. Rank ordered listing of CpG sites contributing
the most to the ordinate discrimination in the NMDS analysis.

| CHR | POS | DOMAIN | UniProt | GO.terms |
|---|---|---|---|---|
| chr3 | 120626543 | upstr | C9JQS3 | negative.regulation.; exocytosis; syntaxin.binding; regulate |
| chr6 | 139349539 | upstr | Q5SZC9 | unk |
| chr12 | 81330338 | upstr | H0YI92 | L27.domain.binding; inner.ear.developmen; exocytosis; synapt |
| chr11 | 61451702 | intron | F5GY58 | endocannabinoid.sign; diacylglycerol.catab; neurotransmitter |
| chr22 | 37680206 | Na | H0Y724 | regulation.of.ARF.pr; ARF.guanyl-nucleotid; regulation.of.ce |
| chr16 | 23653028 | intron | I3L0U8 | antigen.processing.a; |
| chr17 | 38716331 | intron | J3KTN5 | regulation.of.dendri; lymphocyte.migration; response.to.nitr |
| chr14 | 38071393 | intron | Q3SY87 | protein.binding; |
| chr3 | 184302159 | intron | H7C2X0 | binding; positive.regulation.; translational.initia; astrocy |
| chr6 | 10695540 | intron | Q9NWT1 | negative.regulation.; protein.binding |
| chr20 | 48730218 | intron | A0A0A0MSL3 | regulation.of.DNA.re; nucleotide-binding.o; nucleotide-bindi |
| chr3 | 33482718 | intron | C9JWL3 | viral.genome.replica; regulation.of.transc; angiogenesis; ne |
| chr20 | 48553776 | intron | Q9Y508 | spermatogenesis; metal.ion.binding; multicellular.organi; ce |
| chr13 | 48612261 | intron | Q9NV35 | 8-oxo-7; nucleobase-containin; nucleobase-containin; GTP.cat |
| chr15 | 60297395 | exon | Q99853 | cell.migration.in.di; mammary.gland.lobule; mammillothalamic |
| chr11 | 64684954 | upstr | E9PQN5 | binding; protein.phosphatase.; signal.transduction; activati |
| chr18 | 77960570 | exon | K7ELH1 | cell.junction.assemb; cell-cell.junction.o; tight.junction.a |
| chr11 | 41259310 | intron | E9PLP4 | regulation.of.axonog; protein.binding |
| chr12 | 50616434 | 5utr | F8VVQ7 | zinc.ion.binding; actin.monomer.bindin; negative.regulation. |
| chr3 | 155945555 | exon | H7C4S9 | potassium.channel.re; synaptic.transmissio; voltage-gated.po |
| chr11 | 130319714 | exon | Q8TE58 | zinc.ion.binding; proteolysis; metalloendopeptidase |
| chr20 | 43513977 | upstr | Q4VY20 | phosphoserine.bindin; negative.regulation.; cytoplasmic.sequ |
| chr14 | 29235148 | 5utr | P55316 | axon.midline.choice.; central.nervous.syst; forebrain.develo |
| chr3 | 149689478 | exon | C9JQ45 | regulation.of.synapt; negative.regulation.; positive.regulat |
| chr12 | 55247863 | intron | Q96DR8 | post-translational.p; cellular.protein.met; O-glycan.process |
| chr11 | 31846870 | intron | H0YDA4 | in.utero.embryonic.d; camera-type.eye.deve; calcium.ion.bind |
| chr14 | 68286569 | 5utr | A0A0A0MS18 | DNA-dependent.ATPase; DNA.metabolic.proces; DNA.binding; ATP |
| chr15 | 75401874 | intron | H3BU63 | phosphopantothenoyle; coenzyme.biosyntheti; pantothenate.met |
| chr18 | 29523502 | exon | J3QR00 | ER.to.Golgi.vesicle-mediated.transport |
| chr2 | 230421733 | intron | Q8NFT8 | glial.cell.different; Notch.receptor.proce; synapse.assembly |
| chr20 | 58515736 | intron | A2A2M7 | unk |
| chr2 | 133426687 | intron | F8WD77 | unk |
| chr20 | 2821334 | upstr | X6RFT7 | unk |
| chr18 | 19284903 | upstr | Q86YT6 | heart.looping; positive.regulation.; blood.vessel.develop; n |
| chr12 | 96253081 | intron | F8W0W6 | histone.mRNA.metabol; termination.of.RNA.p; ncRNA.metabolic. |
| chr15 | 48625023 | intron | H0YMP1 | dUTP.metabolic.proce; dUTP.diphosphatase.a; hydrolase.activi |
| chr11 | 46938767 | intron | E9PNJ5 | synaptic.growth.at.n; receptor.tyrosine.ki; receptor.cluster |
| chr14 | 72980894 | intron | A0A0A0MRA9 | positive.regulation.; regulation.of.G-prot; termination.of.G |
| chr2 | 176947655 | intron | Q03828 | limb.morphogenesis; sequence-specific.DN; regulation.of.tran |
| chr14 | 78227825 | intron | F8WAD5 | unk |

The CpG list has been filtered for functional pathway assignments. CpGs in hypothetical genes or unknown gene domains with unannotated functions were not included in this hard-copy listing, but all CpGs and their scores are retained in the database and are available for further analyses.

TABLE 22
Top 40 Hypermethylated Genes in Grp1. Genes are presented with PFAM
and RefSeq name designations as well as KEGG pathway associations.

| MethyLoad | UniProt | HUGO | Reactome |
|---|---|---|---|
| 5064.4 | Q9NV35 | NUDT15 | Phosphate.bond.hydrolysis.by.NUDT.proteins |
| 2938.2 | O60404 | OR10H3 | Olfactory.Signaling.Pathway |
| 2024.4 | Q8N146 | OR8H3 | Olfactory.Signaling.Pathway |
| 1803.2 | A6NL26 | OR5B21 | Olfactory.Signaling.Pathway |
| 1694.4 | Q8NGS3 | OR1J1 | Olfactory.Signaling.Pathway |
| 1577.8 | Q96RI9 | TAAR9 | G.alpha.(s).signalling.events |
| 1577.7 | Q9H1M4 | DEFB127 | Defensins |
| 1432.6 | Q8NGW1 | OR6B3 | Olfactory.Signaling.Pathway |
| 1402.9 | P12104 | FABP2 | Hormone-sensitive.lipase.(HSL)-mediated.triacylglycerol. |
| 1397.0 | Q8NG97 | OR2Z1 | Olfactory.Signaling.Pathway |
| 1341.2 | Q8NGE3 | OR10P1 | Olfactory.Signaling.Pathway |
| 1333.1 | Q8NGR9 | OR1N2 | Olfactory.Signaling.Pathway |
| 1319.0 | Q6ZNI0 | GCNT7 | O-linked.glycosylation.of.mucins |
| 1254.8 | P61457 | PCBD1 | Phenylalanine.and.tyrosine.catabolism |
| 1227.2 | P00326 | ADH1C | Ethanol.oxidation |
| 1213.1 | P61024 | CKS1B | Cyclin.D.associated.events.in.G1 |
| 1206.8 | Q6P1L8 | MRPL14 | Mitochondrial.translation.initiation |
| 1189.8 | Q8NH60 | OR52J3 | Olfactory.Signaling.Pathway |
| 1178.8 | Q6UXV4 | APOOL | Platelet.degranulation. |
| 1174.1 | P09681 | GIP | G.alpha.(s).signalling.events |
| 1163.4 | Q9NZP0 | OR6C3 | Olfactory.Signaling.Pathway |
| 1161.7 | Q9NPF7 | IL23A | Signaling.by.Interleukins |
| 1156.2 | Q8NGG8 | OR8B3 | Olfactory.Signaling.Pathway |
| 1150.0 | Q6PGQ7 | BORA | Regulation.of.PLK1.Activity.at.G2/M.Transition |
| 1132.0 | Q96FX8 | PERP | TP53.regulates.transcription.of.several.additional.cell. |
| 1098.8 | Q8NH73 | OR4S2 | Olfactory.Signaling.Pathway |
| 1083.2 | Q8NGX9 | OR6P1 | Olfactory.Signaling.Pathway |
| 1064.6 | Q7RTS3 | PTF1A | Regulation.of.gene.expression.in.early.pancreatic.precur |
| 1063.8 | D6R901 | unk | Ub-specific.processing.proteases |
| 1054.5 | Q8NGD5 | OR4K14 | Olfactory.Signaling.Pathway |
| 1045.9 | Q9H2C8 | OR51V1 | Olfactory.Signaling.Pathway |
| 1041.8 | Q9UHA7 | IL36A | Signaling.by.Interleukins |
| 1033.9 | Q7Z7M8 | B3GNT8 | O-linked.glycosylation.of.mucins |
| 1001.9 | P08620 | FGF4 | FGFR3c.ligand.binding.and.activation |
| 977.2 | Q9ULW2 | FZD10 | Class.B/2.(Secretin.family.receptors) |
| 973.6 | Q8NGI7 | OR10V1 | Olfactory.Signaling.Pathway |
| 948.2 | Q96PE6 | ZIM3 | Generic.Transcription.Pathway |
| 940.5 | Q8N688 | DEFB123 | Defensins |
| 911.9 | A6NP11 | ZNF716 | Generic.Transcription.Pathway |
| 902.5 | Q8NGH3 | OR2D3 | Olfactory.Signaling.Pathway |

The gene list has been filtered for functional pathway assignments. Hypothetical genes or genes with unannotated functions were not included in this hard-copy listing, but all genes and their scores are retained in the database and are available for further analyses.

TABLE 23
Top 40 Hypermethylated Genes in Grp2. Genes are presented with PFAM
and RefSeq name designations as well as KEGG pathway associations.

| MethyLoad | UniProt | HUGO | Reactome |
|---|---|---|---|
| −3253.1 | Q8NGC3 | OR10G2 | Olfactory.Signaling.Pathway |
| −3197.0 | Q8NH85 | OR5R1 | Olfactory.Signaling.Pathway |
| −2289.2 | Q30KQ1 | DEFB133 | Defensins |
| −2275.2 | Q8NGK5 | OR52M1 | Olfactory.Signaling.Pathway |
| −2108.9 | Q8NGT0 | OR13C9 | Olfactory.Signaling.Pathway |
| −1778.5 | Q8NGD4 | OR4K1 | Olfactory.Signaling.Pathway |
| −1744.1 | Q8NH69 | OR5W2 | Olfactory.Signaling.Pathway |
| −1698.9 | Q12918 | KLRB1 | Immunoregulatory.interactions.between.a.Lymphoid.and.a.n |
| −1672.6 | Q969M2 | GJA10 | Gap.junction.assembly |
| −1625.6 | O95221 | OR5F1 | Olfactory.Signaling.Pathway |
| −1611.2 | Q8NGA5 | OR10H4 | Olfactory.Signaling.Pathway |
| −1391.2 | Q8NGH7 | OR52L1 | Olfactory.Signaling.Pathway |
| −1359.7 | Q8NG94 | OR11H1 | Olfactory.Signaling.Pathway |
| −1317.2 | C9JJH3 | unk | Ub-specific.processing.proteases |
| −1306.1 | O95222 | OR6A2 | Olfactory.Signaling. Pathway |
| −1213.5 | Q86XQ3 | CATSPER3 | Sperm.Motility.And.Taxes |
| −1211.3 | Q9NYW4 | TAS2R5 | G.alpha.(i).signalling.events |
| −1192.3 | Q969Q0 | RPL36AL | Nonsense.Mediated.Decay.(NMD).independent.of.the.Exon.Ju |
| −1189.8 | Q5TF39 | MFSD4B | Na+-dependent.glucose.transporters |
| −1183.1 | Q9GZK3 | OR2B2 | Olfactory.Signaling.Pathway |
| −1176.3 | Q9BYW3 | DEFB126 | Defensins |
| −1175.2 | Q96PL1 | SCGB3A2 | Scavenging.by.Class.A.Receptors |
| −1096.4 | Q8NHB1 | OR2V1 | Olfactory.Signaling.Pathway |
| −1092.4 | Q96RA2 | OR7D2 | Olfactory.Signaling.Pathway |
| −1082.1 | Q30KQ7 | DEFB113 | Defensins |
| −1080.4 | Q8NGD1 | OR4N2 | Olfactory.Signaling.Pathway |
| −1075.4 | Q9Y6L6 | SLCO1B1 | Defective.SLCO1B1.causes.hyperbilimbinemia,.Rotor.type. |
| −1070.2 | Q96FJ2 | DYNLL2 | Intraflagellar.transport |
| −1046.3 | Q9Y2Y1 | POLR3K | RNA.Polymerase.III.Chain.Elongation |
| −1045.2 | Q8NGM9 | OR8D4 | Olfactory.Signaling.Pathway |
| −1023.9 | Q8NGQ4 | OR10Q1 | Olfactory.Signaling.Pathway |
| −982.8 | P14652 | HOXB2 | Activation.of.anterior.HOX.genes.in.hindbrain.developmen |
| −969.4 | Q8NGP6 | OR5M8 | Olfactory.Signaling.Pathway |
| −957.6 | Q9UQK1 | PPP1R3C | Myoclonic.epilepsy.of.Lafora |
| −951.1 | P01563 | IFNA2 | TRAF6.mediated.IRF7.activation |
| −919.7 | Q7L3B6 | CDC37L1 | Platelet.degranulation. |
| −912.2 | Q9H082 | RAB33B | Intra-Golgi.traffic |
| −900.2 | A6NL08 | OR6C75 | Olfactory.Signaling.Pathway |
| −888.4 | P61952 | GNG11 | G.alpha.(q).signalling.events |
| −875.8 | Q16552 | IL17A | Interleukin-17.signaling |

The gene list has been filtered for functional pathway assignments. Hypothetical genes or genes with unannotated functions were not included in this hard-copy listing, but all genes and their scores are retained in the database and are available for further analyses.

TABLE 24
Top 30 Hypermethylated Pathways in Grp1. Pathways are rank ordered by
methylation load score (ΔML) and include a list of the top 10 scoring
genes contributing to the pathways load value. The gene list is organized
in decreasing numerical order of the individual gene ΔML scores.

| MethyLoad | Pathway | Genes |
|---|---|---|
| 830.3 | Fructose.mannose.metabolism | Q9NQ88 |
| 704.7 | Lipoic.acid.metabolism | Unk |
| 586.9 | Meiosis.yeast | Q14566 |
| 518.1 | Twocomponent.system | Unk |
| 441.8 | Base.excision.repair | unk |
| 406.8 | Biosynthesis.ansamycins | Q9H0I9 |
| 394.1 | Atrazine.degradation | unk |
| 386.1 | Apoptosis | Q14249 |
| 381.7 | Phototransduction.fly | unk |
| 333.9 | Cell.cycle.yeast | unk |
| 324.1 | Inositol.phosphate.metabolism | Q8NFU5 |
| 308.5 | Primary.bile.acid.biosynthesis | O95992 |
| 307.2 | JakSTAT.signaling.pathway | unk |
| 298.4 | Glycerophospholipid.metabolism | Q9Y259 |
| 274.0 | Amino.sugar.nucleotide.sugar.m | Q8TBE9 |
| 260.2 | Synaptic.vesicle.cycle | Q7Z7G2 |
| 239.4 | Prostate.cancer | Q12778 |
| 236.6 | Prion.diseases | P10643, P11021 |
| 232.8 | Pyrimidine.metabolism | P56597, Q02127 |
| 230.3 | Nucleotide.excision.repair | R4GMW8, P41208, Q6ZYL4 |
| 229.5 | mTOR.signaling.pathway | Q7L523, Q9NX09 |
| 224.0 | Mismatch.repair | unk |
| 213.2 | Glycosylphosphatidylinositolan | unk |
| 211.9 | Terpenoid.backbone.biosynthesi | O75844, Q9BXS1 |
| 204.5 | Novobiocin.biosynthesis | P17735 |
| 203.1 | Viral.carcinogenesis | Q14839, A0A0A0MSJ9, P51679 |
| 198.5 | Selenocompound.metabolism | O43252 |
| 192.2 | Malaria | P07996, Q12918 |
| 180.1 | Sphingolipid.metabolism | Q8TDN7, Q16739, Q9BX95, Q8IWX5 |
| 179.2 | Pathogenic.Escherichia.coli.in | Q9H4B7, P68371, Q9BVA1, Q6PEY2, Q13885 |

Gene order within each pathway is determined by decreasing methylation load scores (_ML). The first gene in the list contributes the most to the pathway methylation score.

TABLE 25
Top 30 Hypermethylated Pathways in Grp2. Pathways are rank ordered by
methylation load score (ΔML) and include a list of the top 10 scoring
genes contributing to the pathways load value. The gene list is organized
in decreasing numerical order of the individual gene ΔML scores.

| MethyLoad | Pathway | Genes |
|---|---|---|
| −875.8 | Rheumatoid.arthritis | Q16552 |
| −748.0 | Starch.sucrose.metabolism | Q6PCE3 |
| −490.8 | B.cell.receptor.signaling.path | Q9UN19 |
| −466.9 | Reninangiotensin.system | unk |
| −444.6 | Tryptophan.metabolism | unk |
| −362.4 | Other.glycan.degradation | Q9Y3R4, P04066 |
| −345.9 | Endocrine.other.factorregulate | unk |
| −342.1 | Autoimmune.thyroid.disease | A0A0R4J2F0 |
| −339.8 | Bisphenol.degradation | Unk |
| −338.0 | Oocyte.meiosis | Q9UQE7 |
| −320.4 | Melanogenesis | P14679, Q01726 |
| −309.9 | Arrhythmogenic.right.ventricul | P17302 |
| −308.2 | Phagosome | Q5KU26 |
| −290.1 | Osteoclast.differentiation | Q9HBY0 |
| −277.7 | Butanoate.metabolism | P0C7M7 |
| −276.7 | Complement.coagulation.cascade | P07204, P00748, P01008, P05160 |
| −270.7 | Streptomycin.biosynthesis | O95455 |
| −267.2 | RNA.polymerase | Q3B726 |
| −263.6 | Metabolism.xenobiotics.by.cyto | O95154 |
| −256.1 | Pancreatic.cancer | unk |
| −247.7 | Shigellosis | P60673 |
| −204.8 | Basal.cell.carcinoma | P12643 |
| −203.9 | Hepatitis.B | P14780, Q13233, O43889, P60484 |
| −201.8 | Small.cell.lung.cancer | P33552, P61024 |
| −197.8 | Chemokine.signaling.pathway | O00626, P10720, Q9Y4X3, O43927, |
| | | P80075, P22362, P47992, P02776 |
| −197.8 | Glycine.serine.threonine.metab | unk |
| −192.9 | Folate.biosynthesis | P35270, Q92820 |
| −192.6 | Hematopoietic.cell.lineage | P06126, P07359, P01588, P15813, |
| | | P29016, P14770 |
| −186.1 | Pantothenate.CoA.biosynthesis | O95497 |
| −184.6 | Gastric.acid.secretion | Q6AI14, P78508, Q9Y6J6, P61278 |

Gene order within each pathway is determined by decreasing methylation load scores (_ML). The first gene in the list contributes the most to the pathway methylation score.

TABLE 26
Top 40 CpG Sites by P-value. Rank ordered listing of CpG sites in known UniProt
genes are sorted by P-values adjusted for false discovery rate (FDR) threshold.
Site-specific dispersion was estimated to equalize CpG variances. A Likelihood
Ratio Test was used with a defined one-way ANOVA model for pairwise tests.

| CpG Site | logFC | FDR adj P-val | Response | Gene | Description |
|---|---|---|---|---|---|
| chr20.0062282831 | −1.77 | 4.24e−19 | down | ENSG00000197457 | NP.001263239 |
| chr3.0119422009 | 1.90 | 1.71e−16 | up | H7C5C8 | — |
| chr10.0104182130 | 1.46 | 4.72e−10 | up | FBXL15 | F-box and leucine rich |
| | | | | | repeat |
| chr16.0031227255 | −2.11 | 5.42e−10 | down | ENSG00000177238 | NP.001008275 |
| chr9.0101559153 | 1.27 | 2.19e−08 | up | ENSG00000165138 | — |
| chr12.0002862275 | −2.02 | 4.79e−08 | down | ENSG00000256150 | — |
| chr16.0029460831 | 1.15 | 8.03e−08 | up | ENSG00000198106 | — |
| chr11.0118306730 | −1.55 | 1.07e−07 | down | ENSG00000118058 | — |
| chr12.0113573398 | −1.74 | 1.70e−07 | down | ENSG00000111344 | — |
| chr14.0069261542 | −2.31 | 3.94e−07 | down | ENSG00000185650 | — |
| chr15.0101300081 | −1.66 | 7.51e−07 | down | ENSG00000259579 | NP.078984 |
| chr3.0184302159 | −1.67 | 1.01e−06 | down | H7C2X0 | — |
| chr20.0041428080 | 1.10 | 1.26e−06 | up | B1AJS0 | — |
| chr12.0005885813 | 1.05 | 1.84e−06 | up | F5GXT3 | — |
| chr2.0177014555 | −2.09 | 2.66e−06 | down | ENSG00000128652 | NP.008829 |
| chr13.0048612261 | −1.57 | 2.71e−06 | down | NUDT15 | nudix hydrolase 15 |
| chr12.0127210936 | −1.40 | 6.91e−06 | down | ENSG00000189238 | NP.001273148 |
| chr6.0010695540 | −1.61 | 7.28e−06 | down | PAK1IP1 | PAK1 interacting protein 1 |
| chr3.0065583873 | 1.57 | 8.97e−06 | up | ENSG00000151276 | — |
| chr20.0004155798 | 0.990 | 9.09e−06 | up | Q5TE25 | — |
| chr18.0077960570 | −1.50 | 9.32e−06 | down | K7ELH1 | — |
| chr11.0060971100 | 1.20 | 1.18e−05 | up | ENSG00000229859 | — |
| chr11.0067173421 | −1.17 | 2.82e−05 | down | F5H037 | — |
| chr22.0037680206 | −1.90 | 4.01e−05 | down | H0Y724 | — |
| chr2.0225266367 | 2.00 | 9.37e−05 | up | ENSG00000124019 | — |
| chr3.0107308392 | 1.80 | 9.37e−05 | up | H7C1Q0 | — |
| chr22.0028542468 | 1.05 | 9.37e−05 | up | B0QYP4 | — |
| chr2.0189618980 | 0.963 | 9.55e−05 | up | ENSG00000223523 | — |
| chr20.0031592073 | 0.850 | 9.55e−05 | up | Q5TDX8 | — |
| chr16.0001877823 | −1.53 | 0.000 | down | ENSG00000180185 | NP.112485 |
| chr12.0050279079 | −1.13 | 0.000 | down | F8VV65 | — |
| chr1.0015251350 | 0.672 | 0.000 | up | ENSG00000189337 | NP.001018001 |
| chr2.0242004042 | 1.39 | 0.000 | up | B5MEF5 | — |
| chr6.0126661739 | −2.22 | 0.000 | down | ENSG00000203760 | — |
| chr17.0080708405 | −1.09 | 0.000 | down | FN3K | fructosamine 3 kinase |
| chr3.0120626543 | −2.11 | 0.000 | down | C9JQS3 | — |
| chr16.0056671862 | 1.40 | 0.000 | up | ENSG00000205362 | NP.005937 |
| chr19.0020387066 | 1.28 | 0.000 | up | ENSG00000267383 | NP.009069 |
| chr21.0045721062 | −1.46 | 0.000 | down | ENSG00000141959 | — |
| chr3.0124976593 | 1.28 | 0.000 | up | ENSG00000221955 | — |

logFC = log[2] of fold change; Response = up or down in Grp2 relative to Grp1.

TABLE 27
Top 1000 CpG sites used used for prediction of risk
of invasiveness in blind study and ranked by p-value.

| CHRPOS | logFC | PValue |
|---|---|---|
| chr20.0005100198 | 1.42179995 | 2.24E−13 |
| chr3.0116170128 | −1.1460506 | 5.32E−13 |
| chr2.0136583332 | 0.89282221 | 5.50E−12 |
| chr18.0003771925 | −2.0448811 | 1.39E−10 |
| chr14.0024615469 | −2.2607198 | 8.23E−10 |
| chr19.0005075863 | −1.1244165 | 8.82E−10 |
| chr5.0056352628 | 0.84421582 | 2.32E−09 |
| chr7.0131268894 | −1.0905434 | 3.02E−09 |
| chrX.0047343019 | 0.74231529 | 4.86E−09 |
| chr3.0139086222 | −0.7729874 | 9.29E−09 |
| chr9.0111238265 | −0.7120328 | 1.51E−08 |
| chr7.0028605794 | −1.0972812 | 1.81E−08 |
| chr12.0133443239 | −1.1540247 | 2.19E−08 |
| chr13.0079551173 | 0.82890047 | 2.38E−08 |
| chr22.0046305438 | 0.89464641 | 2.54E−08 |
| chr15.0097151927 | 1.1072284 | 3.53E−08 |
| chr11.0048200842 | 0.73716749 | 4.48E−08 |
| chr2.0009898288 | 0.99226365 | 4.50E−08 |
| chr2.0121118677 | 0.63220566 | 4.70E−08 |
| chr17.0070760064 | 0.65880467 | 5.57E−08 |
| chr20.0007041339 | 0.96258622 | 6.08E−08 |
| chr2.0229878578 | 0.70667337 | 6.17E−08 |
| chr6.0167651348 | −1.5712132 | 7.21E−08 |
| chr3.0159375789 | 0.80887734 | 7.61E−08 |
| chr16.0021610035 | 2.4050392 | 7.85E−08 |
| chr13.0058888451 | 0.7715333 | 8.75E−08 |
| chr22.0041761752 | 0.91449814 | 8.83E−08 |
| chr16.0074460552 | 0.9557071 | 1.01E−07 |
| chr8.0000224441 | −0.9939628 | 1.04E−07 |
| chr11.0045197654 | 0.73057185 | 1.50E−07 |
| chr7.0004020430 | 0.73295998 | 1.55E−07 |
| chr11.0044338492 | −1.1433836 | 1.66E−07 |
| chr6.0008173236 | 0.70371449 | 1.86E−07 |
| chr17.0006605234 | −1.06738 | 2.21E−07 |
| chr6.0127166748 | 0.71074302 | 3.33E−07 |
| chr5.0107735329 | 0.77293713 | 3.35E−07 |
| chr12.0098725299 | 0.94872166 | 3.63E−07 |
| chrX.0145243680 | 0.55365716 | 3.97E−07 |
| chr14.0103403232 | 0.66924657 | 4.58E−07 |
| chr16.0004816546 | 1.2271846 | 4.59E−07 |
| chr5.0000082900 | −0.7396516 | 4.65E−07 |
| chr2.0048943971 | 0.57157878 | 4.66E−07 |
| chr12.0081923492 | 0.7861611 | 5.47E−07 |
| chr19.0055836171 | −0.7943521 | 5.60E−07 |
| chr17.0077835872 | 0.87262222 | 6.81E−07 |
| chr1.0156416942 | 0.49841039 | 7.47E−07 |
| chr3.0183771836 | 0.70394904 | 7.58E−07 |
| chr5.0004939235 | −0.7432739 | 8.15E−07 |
| chr5.0137610361 | −1.0255363 | 8.49E−07 |
| chr2.0237602634 | −0.7307519 | 8.50E−07 |
| chr9.0137961816 | −0.7393457 | 8.64E−07 |
| chr16.0075145905 | 1.418913 | 9.57E−07 |
| chr18.0055627613 | 0.72690684 | 9.78E−07 |
| chr6.0164722394 | 0.73010958 | 1.00E−06 |
| chr20.0036299083 | 0.7096724 | 1.01E−06 |
| chr11.0122900154 | 0.69933001 | 1.03E−06 |
| chr6.0046295219 | 0.79927637 | 1.08E−06 |
| chr16.0058529353 | −1.2677448 | 1.09E−06 |
| chr12.0122230944 | −1.2872705 | 1.14E−06 |
| chr5.0142142127 | −0.8478472 | 1.14E−06 |
| chr3.0061703710 | −0.6220134 | 1.15E−06 |
| chr14.0094286835 | 0.71267469 | 1.17E−06 |
| chr19.0007936720 | 1.29180551 | 1.31E−06 |
| chr20.0037591461 | −1.6278807 | 1.39E−06 |
| chr11.0062767705 | −0.7265815 | 1.44E−06 |
| chr2.0233308839 | 0.66255665 | 1.54E−06 |
| chr4.0045155506 | 0.54310837 | 1.57E−06 |
| chr9.0000159680 | −1.2515395 | 1.63E−06 |
| chr13.0048449705 | −0.7572854 | 1.78E−06 |
| chr20.0059026704 | −1.1675921 | 1.84E−06 |
| chr15.0059817268 | −0.8535793 | 1.89E−06 |
| chr13.0087178246 | 0.89266193 | 1.89E−06 |
| chr3.0126257511 | −0.6324197 | 1.96E−06 |
| chr14.0102783036 | −1.7689019 | 1.96E−06 |
| chr2.0027579337 | −1.1682393 | 1.97E−06 |
| chr7.0116273352 | 0.81598333 | 2.00E−06 |
| chr2.0128663833 | −0.6209 | 2.07E−06 |
| chr5.0100843674 | 0.68437251 | 2.07E−06 |
| chr11.0119724615 | 0.69053413 | 2.08E−06 |
| chr3.0045133805 | 0.66174842 | 2.22E−06 |
| chr16.0076936044 | 0.7279669 | 2.29E−06 |
| chr5.0140870213 | −0.6006419 | 2.30E−06 |
| chr5.0159804189 | 0.7318523 | 2.32E−06 |
| chr11.0125754851 | 0.71063912 | 2.40E−06 |
| chr11.0115624765 | 0.77906024 | 2.44E−06 |
| chr18.0033675411 | 0.74760036 | 2.49E−06 |
| chr16.0029868044 | 0.66920749 | 2.57E−06 |
| chr9.0012612775 | 0.73600706 | 2.68E−06 |
| chr18.0047057931 | 0.67571035 | 2.80E−06 |
| chr7.0042432323 | 0.67178647 | 2.85E−06 |
| chr5.0099220017 | 0.87458057 | 2.96E−06 |
| chr11.0077185588 | 1.19357809 | 3.00E−06 |
| chr7.0130133579 | −1.0839047 | 3.04E−06 |
| chr8.0144976814 | −1.059382 | 3.16E−06 |
| chr11.0065238870 | 0.70757985 | 3.16E−06 |
| chr18.0037258709 | 0.85653168 | 3.27E−06 |
| chr17.0002439051 | −0.7574154 | 3.39E−06 |
| chr9.0115848647 | −1.5003321 | 3.41E−06 |
| chr6.0004647548 | −0.913113 | 3.41E−06 |
| chr11.0038245360 | 0.7180336 | 3.41E−06 |
| chr10.0132675670 | 0.64545599 | 3.41E−06 |
| chr2.0218661279 | 0.64103416 | 3.45E−06 |
| chr2.0036165222 | 0.77704591 | 3.58E−06 |
| chr5.0053096496 | 0.67029349 | 3.60E−06 |
| chr3.0131485356 | 0.85503524 | 3.72E−06 |
| chr20.0051202616 | 0.82505759 | 3.78E−06 |
| chr8.0061326266 | −0.8922994 | 3.79E−06 |
| chr22.0026176223 | 0.68985137 | 3.79E−06 |
| chr20.0048452918 | −0.7392151 | 3.87E−06 |
| chr17.0031989964 | −0.8847264 | 3.93E−06 |
| chr10.0034801856 | 0.61469321 | 4.27E−06 |
| chr8.0032312701 | 0.72032287 | 4.41E−06 |
| chr11.0026638613 | 0.63297 | 4.42E−06 |
| chr15.0079827069 | 0.78895554 | 4.47E−06 |
| chr18.0010781190 | 0.84285991 | 4.48E−06 |
| chr13.0089743682 | 0.67122942 | 4.52E−06 |
| chr10.0089168004 | 0.93459378 | 4.53E−06 |
| chr13.0045652971 | −0.9397673 | 4.76E−06 |
| chr20.0045811892 | −0.7738618 | 4.82E−06 |
| chr5.0090438852 | 0.68125096 | 4.83E−06 |
| chr16.0065543654 | 0.76701261 | 4.85E−06 |
| chr12.0070329407 | 0.71880582 | 4.86E−06 |
| chr15.0096206901 | 0.7755052 | 4.87E−06 |
| chr14.0060388658 | −0.6959306 | 4.94E−06 |
| chr20.0061030211 | −1.5240984 | 5.02E−06 |
| chr18.0022686352 | 0.88560794 | 5.19E−06 |
| chr15.0041075917 | 0.73011358 | 5.24E−06 |
| chr14.0024641797 | 0.8965562 | 5.27E−06 |
| chr3.0100054571 | −0.7327839 | 5.39E−06 |
| chr5.0084824603 | 0.76840845 | 5.53E−06 |
| chr6.0167335873 | 0.70793696 | 5.53E−06 |
| chr10.0099283204 | 0.57109819 | 5.61E−06 |
| chr2.0177258508 | 0.60913373 | 5.72E−06 |
| chr12.0062860430 | 1.20186348 | 5.80E−06 |
| chr5.0172785376 | 0.72865225 | 5.83E−06 |
| chr5.0157435312 | 0.72818783 | 5.95E−06 |
| chr18.0040240768 | 0.67018018 | 5.99E−06 |
| chr12.0123194517 | 0.63960015 | 6.00E−06 |
| chr7.0026838131 | 0.72219973 | 6.01E−06 |
| chr6.0032096599 | 0.94496626 | 6.05E−06 |
| chr9.0139561883 | −0.686308 | 6.29E−06 |
| chr3.0090052788 | 0.68308633 | 6.31E−06 |
| chr10.0124234752 | −0.7283645 | 6.36E−06 |
| chr19.0012271970 | 0.61821339 | 6.47E−06 |
| chr10.0132926074 | −0.8299413 | 6.53E−06 |
| chr10.0087833424 | 0.66297996 | 6.80E−06 |
| chr18.0019760779 | −0.9542326 | 6.82E−06 |
| chr20.0026221421 | 0.74389756 | 6.96E−06 |
| chr5.0113816235 | 0.7586996 | 6.98E−06 |
| chr15.0053965908 | 0.64101162 | 6.99E−06 |
| chr19.0010767724 | −0.8440964 | 7.25E−06 |
| chr3.0023652434 | 0.61929288 | 7.52E−06 |
| chr11.0035110487 | 0.70515095 | 7.59E−06 |
| chr22.0037200902 | 0.70733823 | 7.75E−06 |
| chr11.0030394645 | 0.61008756 | 7.77E−06 |
| chr18.0037578424 | −0.8420757 | 7.90E−06 |
| chr15.0026276337 | 0.64924394 | 8.06E−06 |
| chr19.0050877556 | 0.60873423 | 8.09E−06 |
| chr3.0045349563 | 0.61674908 | 8.15E−06 |
| chr3.0179270960 | 0.58476677 | 8.17E−06 |
| chr12.0113853884 | 0.66782419 | 8.41E−06 |
| chr15.0039286447 | 0.79591802 | 8.52E−06 |
| chr17.0058090391 | −0.5847732 | 8.73E−06 |
| chr1.0154910059 | −0.5000733 | 8.82E−06 |
| chr20.0019006992 | 0.78415689 | 9.20E−06 |
| chr5.0003741039 | −0.6492828 | 9.71E−06 |
| chrX.0095583828 | 0.51462811 | 9.74E−06 |
| chr3.0031900140 | 0.97626505 | 9.82E−06 |
| chr7.0150544980 | 0.62818195 | 9.92E−06 |
| chr9.0133887499 | −1.0632639 | 9.94E−06 |
| chr14.0024713193 | 0.76724873 | 1.00E−05 |
| chr3.0013836751 | 0.700824 | 1.02E−05 |
| chr6.0015480087 | 0.68530161 | 1.03E−05 |
| chr2.0117820185 | 0.5477872 | 1.05E−05 |
| chr12.0072332759 | −1.1227437 | 1.06E−05 |
| chr2.0010092513 | −0.6775732 | 1.08E−05 |
| chr16.0004948620 | 0.76410136 | 1.14E−05 |
| chr18.0010344859 | 0.65708802 | 1.14E−05 |
| chr14.0061909846 | 0.65127534 | 1.15E−05 |
| chr5.0167798272 | 0.72353008 | 1.18E−05 |
| chr2.0129452164 | 0.63085607 | 1.20E−05 |
| chr19.0032122322 | −0.6052487 | 1.20E−05 |
| chr10.0135055821 | 0.59828613 | 1.21E−05 |
| chr13.0032990677 | −0.6604829 | 1.21E−05 |
| chr18.0000318921 | 0.6420835 | 1.23E−05 |
| chr17.0056584557 | 0.65517354 | 1.24E−05 |
| chr18.0044554788 | 1.72193398 | 1.26E−05 |
| chr11.0078870298 | 0.70383423 | 1.28E−05 |
| chr5.0095160390 | −1.423951 | 1.29E−05 |
| chr3.0197676776 | 2.0746083 | 1.32E−05 |
| chr18.0010285057 | 1.31928837 | 1.33E−05 |
| chr9.0113644978 | −1.126327 | 1.33E−05 |
| chr11.0087430751 | −0.7455805 | 1.34E−05 |
| chr2.0237937088 | 0.6817732 | 1.39E−05 |
| chr8.0121030538 | 0.65272437 | 1.39E−05 |
| chr19.0050463970 | −0.6664938 | 1.39E−05 |
| chrX.0038605033 | 0.42236218 | 1.39E−05 |
| chr3.0186079108 | 0.83584855 | 1.42E−05 |
| chr14.0051490944 | 0.7637686 | 1.42E−05 |
| chr10.0115370456 | −0.6105011 | 1.43E−05 |
| chr5.0153190231 | 0.74823284 | 1.44E−05 |
| chr5.0009469414 | 0.7091629 | 1.47E−05 |
| chr22.0030513658 | 0.64434943 | 1.47E−05 |
| chr11.0064141075 | 1.21208203 | 1.50E−05 |
| chr6.0125445349 | −0.5197668 | 1.50E−05 |
| chr17.0036401720 | 0.69457483 | 1.55E−05 |
| chr12.0054217430 | −0.7282837 | 1.55E−05 |
| chr5.0168360471 | 0.56447978 | 1.58E−05 |
| chr22.0040175178 | 0.72636107 | 1.59E−05 |
| chr18.0008153192 | 0.79448612 | 1.60E−05 |
| chr14.0104558980 | −0.4920784 | 1.64E−05 |
| chr2.0045131050 | 0.61850263 | 1.69E−05 |
| chr2.0031137180 | 0.59195585 | 1.70E−05 |
| chr14.0023051593 | −0.5755742 | 1.77E−05 |
| chr8.0070251074 | −0.5959126 | 1.77E−05 |
| chr20.0055924300 | 0.77477989 | 1.77E−05 |
| chr12.0132869926 | 0.66299989 | 1.78E−05 |
| chr6.0033974720 | 0.75180829 | 1.81E−05 |
| chrX.0118491949 | 0.65517163 | 1.85E−05 |
| chr7.0119364870 | −0.5821766 | 1.87E−05 |
| chr11.0079218833 | −0.8889979 | 1.87E−05 |
| chr20.0038615252 | 1.16961941 | 1.89E−05 |
| chr6.0022316628 | 0.672077 | 1.94E−05 |
| chr3.0167446192 | 0.72409551 | 1.94E−05 |
| chr13.0025744474 | −0.6265788 | 1.98E−05 |
| chr18.0008967618 | 0.64674662 | 1.99E−05 |
| chr12.0057608666 | 1.18712866 | 2.02E−05 |
| chrX.0063901420 | 0.53883304 | 2.02E−05 |
| chr22.0044894647 | 0.79731823 | 2.03E−05 |
| chr18.0073417261 | 0.84527471 | 2.05E−05 |
| chr7.0084956359 | 0.66869985 | 2.05E−05 |
| chr18.0024532397 | 0.72142809 | 2.05E−05 |
| chr16.0085377991 | 0.52470179 | 2.06E−05 |
| chr19.0032252343 | −0.6132411 | 2.07E−05 |
| chr18.0037333341 | 0.72332161 | 2.10E−05 |
| chr15.0062364524 | 0.81978089 | 2.10E−05 |
| chr10.0122349007 | 0.5975229 | 2.14E−05 |
| chr19.0044273158 | −0.6421758 | 2.16E−05 |
| chr6.0162260569 | 0.7387621 | 2.16E−05 |
| chr20.0001813479 | −0.7895873 | 2.16E−05 |
| chr6.0044598826 | 0.74785716 | 2.16E−05 |
| chr12.0053690492 | 0.73301751 | 2.18E−05 |
| chr7.0114558533 | 0.66642435 | 2.19E−05 |
| chr10.0027663158 | 0.50367803 | 2.21E−05 |
| chr13.0113393004 | 0.57722642 | 2.25E−05 |
| chr12.0088812781 | 0.73396966 | 2.26E−05 |
| chr15.0029873995 | 0.84952863 | 2.27E−05 |
| chr3.0077544810 | 0.7291718 | 2.28E−05 |
| chr13.0057745722 | 0.83463591 | 2.29E−05 |
| chr13.0094478408 | 0.61091781 | 2.33E−05 |
| chr13.0110921029 | 0.73210605 | 2.35E−05 |
| chr17.0050903002 | 0.55589211 | 2.39E−05 |
| chr7.0118399916 | 0.56107487 | 2.41E−05 |
| chr20.0043423337 | 0.66700441 | 2.42E−05 |
| chr12.0060613750 | 0.70141486 | 2.42E−05 |
| chr11.0004233481 | 0.98622828 | 2.42E−05 |
| chr18.0065707474 | 0.67163249 | 2.43E−05 |
| chr17.0042435592 | −0.5371504 | 2.44E−05 |
| chr12.0117663292 | 0.67995955 | 2.46E−05 |
| chr5.0108056803 | 0.69909423 | 2.47E−05 |
| chrX.0047639461 | 0.4805158 | 2.47E−05 |
| chr13.0086470118 | 0.97624718 | 2.48E−05 |
| chr13.0059951220 | 0.62593416 | 2.52E−05 |
| chr3.0013182915 | 0.48696429 | 2.52E−05 |
| chr16.0065779623 | 0.66463129 | 2.57E−05 |
| chr8.0123692428 | −0.6304996 | 2.57E−05 |
| chr2.0083749925 | −0.621556 | 2.60E−05 |
| chr2.0031912745 | 0.5429896 | 2.64E−05 |
| chr5.0146747249 | 0.8371562 | 2.66E−05 |
| chr5.0034850751 | 0.65267297 | 2.68E−05 |
| chr12.0113881154 | 0.65084345 | 2.69E−05 |
| chr20.0031337087 | −0.6450485 | 2.73E−05 |
| chr19.0051345820 | 0.66315546 | 2.74E−05 |
| chr16.0022628906 | 1.06032211 | 2.74E−05 |
| chr14.0034874938 | 0.86787504 | 2.82E−05 |
| chr16.0022162322 | 0.50718153 | 2.82E−05 |
| chr20.0024545582 | 0.6483386 | 2.83E−05 |
| chr16.0051239999 | 0.76967706 | 2.83E−05 |
| chr14.0086837080 | 0.72790847 | 2.87E−05 |
| chr3.0045730624 | −1.1348577 | 2.87E−05 |
| chr8.0011113648 | 0.54785681 | 2.89E−05 |
| chr5.0017582586 | 2.13401852 | 2.91E−05 |
| chr4.0125269456 | 0.4601591 | 2.94E−05 |
| chr22.0050166610 | −1.4274981 | 3.00E−05 |
| chr3.0191599485 | 0.9379011 | 3.02E−05 |
| chr12.0013480692 | 0.63874404 | 3.03E−05 |
| chr20.0002645380 | 1.37839817 | 3.03E−05 |
| chr4.0154572473 | −0.762115 | 3.04E−05 |
| chr14.0096735677 | 0.79552884 | 3.06E−05 |
| chr20.0003081508 | 0.63818167 | 3.11E−05 |
| chr11.0093475384 | −1.0538671 | 3.18E−05 |
| chr2.0127944138 | −0.7316796 | 3.20E−05 |
| chr16.0077290798 | 0.71394838 | 3.22E−05 |
| chr5.0111943483 | 0.66157324 | 3.31E−05 |
| chr14.0105673580 | 0.60292478 | 3.33E−05 |
| chr3.0004536474 | 1.18245978 | 3.36E−05 |
| chr17.0008534871 | 0.84096157 | 3.37E−05 |
| chr7.0073319413 | 0.52199307 | 3.37E−05 |
| chrX.0102955147 | 0.41763407 | 3.37E−05 |
| chr18.0019538212 | 0.72580881 | 3.39E−05 |
| chr6.0052050745 | 0.62611075 | 3.42E−05 |
| chr7.0118529664 | 0.484139 | 3.43E−05 |
| chr6.0074871011 | 0.79821896 | 3.44E−05 |
| chr20.0049638010 | −1.179746 | 3.52E−05 |
| chr5.0011125653 | 0.56518526 | 3.52E−05 |
| chr5.0062476876 | 0.81475558 | 3.57E−05 |
| chr2.0142741433 | −0.4886771 | 3.64E−05 |
| chr5.0180857365 | 0.58771246 | 3.65E−05 |
| chr18.0020681043 | 0.55257366 | 3.66E−05 |
| chr6.0147480762 | −0.6741294 | 3.72E−05 |
| chr5.0151997255 | 0.59437445 | 3.73E−05 |
| chr2.0209090040 | 0.53083583 | 3.78E−05 |
| chr13.0036140625 | 0.8078307 | 3.78E−05 |
| chr15.0066332769 | 0.683103 | 3.80E−05 |
| chr3.0189770054 | 0.62026038 | 3.82E−05 |
| chr12.0020573645 | 0.62428502 | 3.83E−05 |
| chr16.0011480658 | −0.6851073 | 3.84E−05 |
| chr20.0047542258 | 0.86499833 | 3.86E−05 |
| chr4.0083822472 | −0.9056179 | 3.90E−05 |
| chr20.0022843630 | −0.7686303 | 3.95E−05 |
| chr5.0148540678 | −0.6035131 | 4.00E−05 |
| chr19.0051551164 | −0.7927634 | 4.10E−05 |
| chr22.0036674237 | −0.8048337 | 4.11E−05 |
| chr5.0089575203 | 0.65230132 | 4.15E−05 |
| chr21.0022014524 | 0.46317946 | 4.20E−05 |
| chr3.0042650476 | −0.7421869 | 4.22E−05 |
| chr19.0007922405 | 0.51191565 | 4.23E−05 |
| chr9.0102164104 | 0.70823466 | 4.25E−05 |
| chr3.0054003756 | 0.74048684 | 4.25E−05 |
| chr8.0025546618 | 0.87526386 | 4.27E−05 |
| chr12.0000856233 | 0.72763442 | 4.27E−05 |
| chr17.0044984463 | −0.5763985 | 4.29E−05 |
| chr5.0032042842 | 0.64083331 | 4.31E−05 |
| chr8.0033609677 | 0.61856466 | 4.32E−05 |
| chr4.0148459836 | 0.43460444 | 4.35E−05 |
| chr6.0020323387 | 0.65007173 | 4.36E−05 |
| chr19.0000664734 | 0.68205759 | 4.37E−05 |
| chr6.0029040308 | 0.6661952 | 4.38E−05 |
| chr2.0233623357 | 0.34146213 | 4.40E−05 |
| chr6.0026021290 | −0.9270936 | 4.41E−05 |
| chr13.0063032156 | −0.7541626 | 4.41E−05 |
| chr12.0069480663 | 0.62097894 | 4.41E−05 |
| chr17.0021320889 | 0.50800652 | 4.44E−05 |
| chr8.0095024600 | 0.61617718 | 4.46E−05 |
| chr21.0028306298 | −0.4355619 | 4.53E−05 |
| chr20.0061048321 | −0.7294245 | 4.58E−05 |
| chr17.0008482325 | 0.62234019 | 4.59E−05 |
| chr20.0004519443 | 0.65699312 | 4.59E−05 |
| chr5.0063380472 | −0.5993912 | 4.60E−05 |
| chr3.0125843470 | 0.69531381 | 4.64E−05 |
| chr20.0044576281 | 0.6310868 | 4.65E−05 |
| chr8.0118993090 | −0.8425844 | 4.65E−05 |
| chr12.0000248545 | −1.0664604 | 4.70E−05 |
| chr14.0069475762 | 0.51841343 | 4.70E−05 |
| chr6.0077620916 | 0.62696648 | 4.70E−05 |
| chr11.0083398409 | 0.66369636 | 4.73E−05 |
| chr7.0102248375 | 0.70170087 | 4.73E−05 |
| chr18.0043151423 | 0.67330864 | 4.74E−05 |
| chr7.0135468282 | 0.86163115 | 4.75E−05 |
| chr5.0043514696 | 0.97435005 | 4.76E−05 |
| chr10.0024505612 | −0.5968404 | 4.79E−05 |
| chr20.0036859008 | 0.73075239 | 4.81E−05 |
| chr14.0039778372 | −0.5612771 | 4.83E−05 |
| chr12.0131267493 | 0.58660077 | 4.84E−05 |
| chr3.0169377024 | −1.5196662 | 4.89E−05 |
| chr16.0087289062 | −0.9665907 | 4.90E−05 |
| chr3.0040158531 | 0.61605351 | 4.91E−05 |
| chr18.0075565158 | 0.62751826 | 4.99E−05 |
| chr20.0018370899 | 0.77886302 | 5.02E−05 |
| chr9.0114583135 | 0.51193754 | 5.05E−05 |
| chr15.0032962495 | −0.6329209 | 5.10E−05 |
| chr16.0066766098 | 0.64376193 | 5.11E−05 |
| chr9.0029005710 | 0.77629009 | 5.16E−05 |
| chr2.0030607119 | 0.56524627 | 5.19E−05 |
| chr18.0051546395 | 0.58550457 | 5.22E−05 |
| chr10.0091816436 | 0.66674605 | 5.24E−05 |
| chr10.0071224785 | 0.6145159 | 5.29E−05 |
| chr12.0015307234 | −0.7875691 | 5.38E−05 |
| chr20.0022548751 | −1.8334542 | 5.39E−05 |
| chr10.0104962224 | −1.0513033 | 5.43E−05 |
| chr3.0053381711 | −0.9623727 | 5.44E−05 |
| chr7.0149322908 | −0.8173846 | 5.44E−05 |
| chr7.0135515803 | 0.67817196 | 5.48E−05 |
| chr5.0178699789 | 0.61678996 | 5.49E−05 |
| chr20.0035598854 | 0.57694962 | 5.50E−05 |
| chr8.0109649690 | −0.5482201 | 5.51E−05 |
| chr13.0058906703 | 0.62306459 | 5.53E−05 |
| chr16.0090079951 | 0.74900089 | 5.53E−05 |
| chr11.0002054231 | 0.46873414 | 5.55E−05 |
| chr2.0070521984 | 0.57683822 | 5.55E−05 |
| chr6.0030883001 | 0.76963492 | 5.59E−05 |
| chr2.0036319353 | 0.52718918 | 5.59E−05 |
| chr18.0066419421 | 0.57663911 | 5.61E−05 |
| chr9.0100565156 | 0.62464443 | 5.64E−05 |
| chr14.0085459858 | −0.6667667 | 5.65E−05 |
| chr11.0032605655 | −0.8578852 | 5.71E−05 |
| chr3.0140189640 | −0.6444057 | 5.71E−05 |
| chr15.0095252350 | 0.61120513 | 5.71E−05 |
| chr15.0083349842 | −0.7505995 | 5.73E−05 |
| chr1.0026848449 | 0.29497712 | 5.73E−05 |
| chr13.0028341528 | 0.73577173 | 5.74E−05 |
| chr22.0039513323 | 1.13234519 | 5.75E−05 |
| chr13.0112113122 | −0.7945438 | 5.77E−05 |
| chr8.0119356020 | 0.59807431 | 5.79E−05 |
| chr5.0022033556 | 0.66966543 | 5.80E−05 |
| chr10.0098833234 | 0.68809424 | 5.80E−05 |
| chr11.0035683582 | −1.2733195 | 5.82E−05 |
| chr16.0027958387 | 0.66843552 | 5.83E−05 |
| chr13.0054257876 | 0.72895813 | 5.89E−05 |
| chr20.0002191475 | 0.88490961 | 5.89E−05 |
| chr12.0002891673 | 0.65080261 | 5.91E−05 |
| chr6.0123196131 | 0.50227076 | 5.92E−05 |
| chr19.0048408137 | −1.950993 | 5.92E−05 |
| chr14.0101184774 | 0.61475611 | 5.95E−05 |
| chr17.0058506619 | 0.49810419 | 5.95E−05 |
| chr12.0083143812 | 0.59243512 | 5.96E−05 |
| chr5.0083424175 | 0.75275646 | 5.97E−05 |
| chr12.0131119880 | 0.73884797 | 6.02E−05 |
| chr2.0167233080 | 0.85326733 | 6.02E−05 |
| chr20.0034317445 | 0.68393495 | 6.03E−05 |
| chr13.0045562411 | 0.59892565 | 6.07E−05 |
| chr8.0028516393 | 0.59693948 | 6.09E−05 |
| chr5.0172177982 | 0.486652 | 6.11E−05 |
| chr8.0030267885 | 0.62907153 | 6.12E−05 |
| chr12.0122199533 | −0.7293376 | 6.12E−05 |
| chr10.0101769028 | 0.81829251 | 6.22E−05 |
| chr2.0123651160 | 0.66300834 | 6.24E−05 |
| chr2.0212549100 | 0.60329173 | 6.27E−05 |
| chr5.0001782719 | 0.64118876 | 6.28E−05 |
| chr5.0142150994 | −1.1780579 | 6.30E−05 |
| chr20.0055975261 | 0.57551146 | 6.30E−05 |
| chr2.0184699793 | 0.62526934 | 6.31E−05 |
| chrX.0108896337 | −0.3890609 | 6.35E−05 |
| chr17.0070088109 | 0.66066439 | 6.36E−05 |
| chr19.0002028589 | 0.46705916 | 6.37E−05 |
| chrY.0002515925 | 0.78045788 | 6.37E−05 |
| chr11.0129600966 | 0.74529121 | 6.40E−05 |
| chr12.0059438359 | 0.68692028 | 6.40E−05 |
| chr8.0033329063 | −0.6145708 | 6.42E−05 |
| chr14.0089258276 | −0.7550806 | 6.42E−05 |
| chr17.0067844563 | 0.64665144 | 6.42E−05 |
| chr19.0042117746 | 0.75255846 | 6.43E−05 |
| chr14.0021505537 | 0.69475892 | 6.47E−05 |
| chr2.0236761479 | 0.72919097 | 6.49E−05 |
| chr2.0152416723 | −0.5044885 | 6.51E−05 |
| chr20.0055449579 | −0.6668461 | 6.51E−05 |
| chr12.0071315264 | −0.6989507 | 6.53E−05 |
| chr16.0009707569 | −0.7693812 | 6.55E−05 |
| chr4.0109112025 | 0.41397112 | 6.57E−05 |
| chr11.0127352640 | 0.72051896 | 6.59E−05 |
| chr18.0050420110 | 0.78336499 | 6.62E−05 |
| chr7.0031275537 | 0.63375644 | 6.68E−05 |
| chr5.0135913015 | 0.56396908 | 6.69E−05 |
| chr17.0005538078 | 0.66893536 | 6.72E−05 |
| chr2.0135469656 | −0.6690426 | 6.74E−05 |
| chr9.0140381964 | 0.57367712 | 6.74E−05 |
| chr15.0075100435 | 0.69133801 | 6.77E−05 |
| chr5.0170415953 | 0.67214714 | 6.78E−05 |
| chr15.0094562476 | 0.52029173 | 6.81E−05 |
| chr11.0049873423 | 0.53746295 | 6.84E−05 |
| chr15.0099422149 | 0.50589662 | 6.85E−05 |
| chr4.0009367688 | 1.05573304 | 6.91E−05 |
| chr12.0110588072 | 0.43987747 | 6.92E−05 |
| chr2.0006212409 | −0.5581524 | 6.95E−05 |
| chr5.0134404584 | −0.5438434 | 6.95E−05 |
| chr11.0045095500 | −0.7366137 | 7.00E−05 |
| chr12.0059254299 | 0.71040681 | 7.01E−05 |
| chr5.0126148583 | −0.4940995 | 7.03E−05 |
| chr20.0058089204 | 0.65205833 | 7.06E−05 |
| chr7.0155278976 | 0.52904555 | 7.08E−05 |
| chr18.0042768021 | 0.61547275 | 7.08E−05 |
| chr4.0042486279 | 0.59461629 | 7.16E−05 |
| chr17.0008796141 | 0.56129207 | 7.18E−05 |
| chr18.0020707348 | −0.7100423 | 7.18E−05 |
| chr18.0035045388 | 0.65097702 | 7.19E−05 |
| chr15.0025114024 | 0.55911212 | 7.20E−05 |
| chr15.0023616491 | 0.79448497 | 7.20E−05 |
| chr9.0015037405 | 0.5739414 | 7.22E−05 |
| chr5.0149351479 | 0.6450143 | 7.24E−05 |
| chr4.0153282974 | 0.48668714 | 7.25E−05 |
| chr17.0033475325 | −0.7254451 | 7.25E−05 |
| chr5.0039657149 | 0.66373036 | 7.30E−05 |
| chr3.0053764689 | 0.68278748 | 7.31E−05 |
| chr7.0042051909 | 0.66090356 | 7.33E−05 |
| chr20.0009371692 | 0.80024066 | 7.33E−05 |
| chr12.0090457772 | −0.627399 | 7.37E−05 |
| chr13.0043565901 | 1.19516302 | 7.37E−05 |
| chr16.0054678468 | −0.6381258 | 7.40E−05 |
| chr12.0115160945 | 0.65975924 | 7.41E−05 |
| chr12.0091617026 | 0.60733283 | 7.47E−05 |
| chr15.0037276969 | 0.68729523 | 7.49E−05 |
| chr2.0233541132 | 0.52181393 | 7.52E−05 |
| chr18.0000850358 | 0.70112892 | 7.59E−05 |
| chr8.0048669534 | 0.52877611 | 7.61E−05 |
| chr14.0038930138 | −0.799294 | 7.61E−05 |
| chr2.0075497488 | 0.62682253 | 7.63E−05 |
| chr22.0016954689 | 0.65801998 | 7.65E−05 |
| chr16.0088019305 | −0.5879556 | 7.67E−05 |
| chr13.0069590196 | 0.64140308 | 7.68E−05 |
| chr14.0035870676 | 1.15099062 | 7.70E−05 |
| chr10.0038155813 | 0.6140806 | 7.72E−05 |
| chr2.0106013724 | 0.62535969 | 7.72E−05 |
| chr20.0039043011 | 0.64374059 | 7.72E−05 |
| chr5.0098654376 | 0.64387508 | 7.75E−05 |
| chr12.0115620946 | 0.78795928 | 7.77E−05 |
| chr10.0065589698 | 0.77291632 | 7.78E−05 |
| chr9.0041986627 | 0.83421783 | 7.83E−05 |
| chr19.0004125024 | −0.8022955 | 7.84E−05 |
| chr5.0055893027 | −0.7063398 | 7.86E−05 |
| chr1.0226796571 | −0.5446267 | 7.86E−05 |
| chr14.0039424782 | −0.5222755 | 7.88E−05 |
| chr14.0100251061 | 0.66804197 | 7.91E−05 |
| chr14.0078559726 | 0.71221956 | 7.92E−05 |
| chr17.0065322618 | 0.59502513 | 7.98E−05 |
| chr7.0039275080 | 0.68675165 | 8.03E−05 |
| chr19.0050452406 | 0.5454122 | 8.03E−05 |
| chrX.0079830455 | −0.4329939 | 8.07E−05 |
| chr6.0004020198 | −0.5764275 | 8.08E−05 |
| chr1.0104147243 | 0.33113906 | 8.09E−05 |
| chr20.0049454910 | 0.57191571 | 8.11E−05 |
| chr6.0007835627 | 0.63236399 | 8.11E−05 |
| chr18.0043697516 | 0.68070516 | 8.11E−05 |
| chr17.0033218831 | 0.60394456 | 8.12E−05 |
| chr10.0075754955 | 0.54145842 | 8.13E−05 |
| chr18.0060314213 | 0.66306457 | 8.15E−05 |
| chr11.0062904067 | 0.68687927 | 8.16E−05 |
| chr2.0008793681 | 0.59946059 | 8.17E−05 |
| chr7.0110733759 | 0.63885347 | 8.17E−05 |
| chr5.0081084345 | 0.59591137 | 8.18E−05 |
| chr3.0187848777 | 0.6512515 | 8.18E−05 |
| chr20.0034859230 | −0.6855537 | 8.19E−05 |
| chr18.0046473590 | 0.66069171 | 8.27E−05 |
| chr8.0103819829 | −0.8991484 | 8.28E−05 |
| chr9.0138035748 | 0.5933991 | 8.30E−05 |
| chr7.0028399657 | 0.53335498 | 8.33E−05 |
| chr22.0031766358 | 0.67143522 | 8.35E−05 |
| chr8.0061909850 | 0.58395448 | 8.36E−05 |
| chr4.0036324115 | 0.57774857 | 8.36E−05 |
| chr15.0098936810 | −0.651508 | 8.39E−05 |
| chr7.0037809913 | 0.57936561 | 8.43E−05 |
| chr5.0142134978 | 0.56357222 | 8.48E−05 |
| chr9.0118808657 | 0.67937379 | 8.51E−05 |
| chr11.0064579326 | 0.54819959 | 8.52E−05 |
| chr11.0004624604 | −0.5799899 | 8.53E−05 |
| chr20.0059665861 | −1.0120943 | 8.56E−05 |
| chr14.0102341669 | −0.8881467 | 8.62E−05 |
| chr13.0101852185 | 0.70190538 | 8.63E−05 |
| chr11.0047448895 | 0.64272966 | 8.63E−05 |
| chr9.0113635818 | −0.8020933 | 8.64E−05 |
| chr12.0122059235 | −0.4530727 | 8.65E−05 |
| chr19.0043174131 | 0.56959004 | 8.70E−05 |
| chr5.0055649454 | 0.51574574 | 8.71E−05 |
| chr5.0149516102 | 0.53143141 | 8.75E−05 |
| chr3.0147367230 | 0.61624879 | 8.76E−05 |
| chrY.0059011243 | −1.3848427 | 8.77E−05 |
| chr13.0054953014 | −0.4875467 | 8.78E−05 |
| chr22.0030179859 | 0.57848354 | 8.82E−05 |
| chr12.0123988785 | 0.56303354 | 8.85E−05 |
| chr3.0045779502 | 0.64187396 | 8.86E−05 |
| chr12.0056281966 | 0.58702132 | 8.92E−05 |
| chr9.0029152845 | 0.6556145 | 8.94E−05 |
| chr21.0011059841 | 0.57923866 | 8.95E−05 |
| chr17.0034905342 | 0.67033712 | 8.96E−05 |
| chr6.0036571931 | 0.65081736 | 8.98E−05 |
| chr5.0167589692 | −0.5050013 | 9.06E−05 |
| chr22.0028130252 | −0.5963267 | 9.06E−05 |
| chr2.0237612447 | −0.5853852 | 9.07E−05 |
| chr2.0076804932 | −0.5067826 | 9.09E−05 |
| chr12.0054380350 | 0.78792075 | 9.09E−05 |
| chr19.0051224527 | −0.9224439 | 9.11E−05 |
| chr3.0142163374 | −0.6590653 | 9.11E−05 |
| chr2.0013812505 | 0.61095335 | 9.12E−05 |
| chr5.0044902893 | 0.63612183 | 9.17E−05 |
| chr12.0002444470 | −1.0181507 | 9.17E−05 |
| chrX.0046982420 | 0.45045936 | 9.17E−05 |
| chr7.0097988180 | 0.5606052 | 9.20E−05 |
| chr17.0001159396 | 0.63421331 | 9.21E−05 |
| chr6.0119109856 | 0.64560805 | 9.21E−05 |
| chr16.0005573968 | −1.1641581 | 9.21E−05 |
| chr2.0154839425 | 0.49219521 | 9.24E−05 |
| chr11.0045780767 | 0.4583319 | 9.26E−05 |
| chr19.0033785244 | 0.53777841 | 9.29E−05 |
| chr4.0011193230 | −0.5672935 | 9.36E−05 |
| chr6.0017087114 | 0.68578808 | 9.40E−05 |
| chr6.0159864708 | −0.8378025 | 9.41E−05 |
| chr1.0068164587 | 0.28385524 | 9.45E−05 |
| chrX.0041105767 | −0.4382789 | 9.45E−05 |
| chr3.0072648316 | 0.61943138 | 9.46E−05 |
| chr2.0108882933 | 0.56689005 | 9.47E−05 |
| chr7.0000812922 | 0.5020507 | 9.51E−05 |
| chr17.0071955191 | 0.55357976 | 9.54E−05 |
| chr13.0033652848 | 0.56502063 | 9.59E−05 |
| chr20.0041047388 | 0.74592846 | 9.61E−05 |
| chr8.0144977342 | −0.7478489 | 9.64E−05 |
| chr12.0107895401 | 0.60987928 | 9.67E−05 |
| chr7.0055632695 | 0.47794788 | 9.68E−05 |
| chr16.0016465180 | 0.68189572 | 9.70E−05 |
| chr7.0063031682 | −0.6188045 | 9.71E−05 |
| chr5.0017515446 | −0.5968351 | 9.72E−05 |
| chr13.0084070780 | 0.65562271 | 9.75E−05 |
| chr11.0038974484 | 0.62980791 | 9.83E−05 |
| chr8.0058911722 | −0.9556826 | 9.86E−05 |
| chr20.0002078305 | −0.5341574 | 9.93E−05 |
| chr13.0110817281 | 0.63474386 | 9.93E−05 |
| chr11.0007782193 | 0.69779861 | 9.93E−05 |
| chr17.0000751538 | −0.6227767 | 9.97E−05 |
| chr2.0023901765 | 0.480467 | 0.00010105 |
| chr6.0009469987 | 0.59233009 | 0.00010105 |
| chr4.0008877517 | 0.97584417 | 0.0001017 |
| chr9.0067622317 | −0.695423 | 0.00010246 |
| chr3.0096068414 | −0.5138859 | 0.0001026 |
| chr13.0071999222 | 0.58406872 | 0.00010295 |
| chr15.0083222696 | 0.6183663 | 0.00010366 |
| chr16.0028062653 | 0.62028283 | 0.00010418 |
| chr19.0048409220 | −0.8388879 | 0.00010447 |
| chr13.0049030680 | −0.4345108 | 0.00010457 |
| chr18.0068103824 | −0.7154778 | 0.0001047 |
| chr7.0005330924 | 0.54118864 | 0.00010501 |
| chr3.0170583667 | 0.690587 | 0.00010525 |
| chrX.0128267707 | 0.4689614 | 0.00010564 |
| chr7.0078710364 | 0.50497275 | 0.00010566 |
| chr17.0066676963 | 0.55883737 | 0.00010571 |
| chr13.0047980770 | 0.59133291 | 0.00010767 |
| chr20.0014734886 | 0.66867799 | 0.00010786 |
| chr6.0010317273 | −0.6907944 | 0.00010874 |
| chr7.0016535085 | 0.61010717 | 0.00010897 |
| chr5.0173765047 | 0.82871849 | 0.00010898 |
| chr6.0055808343 | 0.54733713 | 0.00010904 |
| chr3.0119772040 | 0.65516197 | 0.0001096 |
| chr6.0011484137 | 0.55909608 | 0.00010971 |
| chr11.0121835399 | 0.68833914 | 0.00011086 |
| chr10.0098370978 | −0.6034927 | 0.00011104 |
| chr16.0070474071 | 0.69746261 | 0.0001115 |
| chr20.0054277631 | 0.67369977 | 0.00011169 |
| chr5.0006249356 | −0.6053046 | 0.0001118 |
| chr16.0009523016 | −0.6344105 | 0.00011195 |
| chr7.0101575579 | 0.5411699 | 0.00011214 |
| chr22.0049741007 | 0.67772953 | 0.00011228 |
| chr17.0005370938 | −0.6502286 | 0.00011244 |
| chr9.0080986992 | −1.1403954 | 0.00011248 |
| chr15.0061006191 | 0.5975277 | 0.00011342 |
| chr5.0083700789 | 0.60016101 | 0.00011351 |
| chr6.0069073378 | 0.51930587 | 0.00011366 |
| chr16.0009820336 | −0.8193436 | 0.00011394 |
| chr3.0079708180 | 0.7706252 | 0.00011426 |
| chr5.0155367004 | 0.66921546 | 0.00011439 |
| chr17.0002446121 | −0.613246 | 0.00011506 |
| chr3.0141090474 | 0.49362458 | 0.00011517 |
| chr5.0109507022 | 0.6634853 | 0.00011525 |
| chr2.0099238844 | 0.60620476 | 0.00011533 |
| chr15.0086412794 | 0.60177692 | 0.00011568 |
| chr6.0157050206 | 0.63240138 | 0.00011596 |
| chr5.0056054557 | 0.64960701 | 0.00011609 |
| chr6.0117693607 | −0.8723993 | 0.00011665 |
| chr7.0147029810 | 0.60518433 | 0.00011681 |
| chrX.0035108412 | 0.42372003 | 0.00011686 |
| chr10.0135026776 | −0.5914297 | 0.0001174 |
| chr2.0239799598 | 0.51901779 | 0.00011746 |
| chr17.0046115430 | 0.75338073 | 0.00011776 |
| chr9.0018040487 | −0.5619297 | 0.0001185 |
| chr4.0002807713 | 0.54046499 | 0.00011863 |
| chr3.0015178538 | −0.6661274 | 0.00011893 |
| chr4.0143720070 | −0.7470219 | 0.00011905 |
| chr12.0122444432 | 0.58300005 | 0.00011939 |
| chr20.0002004019 | −1.3899023 | 0.0001195 |
| chr7.0133317060 | 0.7241805 | 0.00011992 |
| chr3.0182287894 | 0.75842933 | 0.00012029 |
| chr17.0005114675 | 0.62360116 | 0.00012033 |
| chr14.0034056778 | 0.4916125 | 0.00012059 |
| chr2.0214338606 | 0.55806362 | 0.00012063 |
| chr14.0021409713 | −0.6257232 | 0.0001207 |
| chr10.0047683393 | −0.5689799 | 0.00012071 |
| chr10.0075174846 | 0.74274529 | 0.00012084 |
| chr14.0075637877 | −0.5280271 | 0.00012089 |
| chr2.0001990242 | 0.59183402 | 0.00012092 |
| chr7.0126787341 | 0.61481934 | 0.00012121 |
| chr10.0013294841 | −0.5491947 | 0.00012122 |
| chr2.0065456814 | −0.5206551 | 0.00012142 |
| chr5.0028769509 | 0.62715393 | 0.00012165 |
| chr6.0155652245 | 0.44235243 | 0.00012237 |
| chr17.0075818713 | −0.5360731 | 0.00012273 |
| chr14.0034139300 | 0.62600911 | 0.00012294 |
| chr5.0023517751 | 0.60046928 | 0.00012308 |
| chr18.0036715997 | 0.62303635 | 0.00012339 |
| chr5.0118688297 | 0.66245605 | 0.00012339 |
| chr3.0052758173 | 0.59771772 | 0.00012391 |
| chr18.0056709616 | −0.6472826 | 0.00012406 |
| chr19.0000677860 | −0.5460962 | 0.00012425 |
| chr5.0046374787 | 0.58672441 | 0.00012444 |
| chr1.0203302184 | −0.3286749 | 0.00012559 |
| chr10.0082189279 | −0.4191589 | 0.00012629 |
| chr5.0024572294 | 0.62758039 | 0.00012656 |
| chr10.0091378308 | 0.44614534 | 0.00012678 |
| chr13.0031552310 | 0.50576834 | 0.00012705 |
| chr18.0072838054 | −0.606763 | 0.00012719 |
| chr6.0019727622 | 0.60075024 | 0.0001274 |
| chr18.0007771148 | 1.04348992 | 0.00012744 |
| chr4.0017767338 | 0.70486517 | 0.00012766 |
| chr22.0047515249 | 0.45593574 | 0.0001281 |
| chr17.0021214479 | −0.7303715 | 0.0001289 |
| chr2.0008019516 | 0.59953683 | 0.00012916 |
| chr22.0038350894 | 0.51557308 | 0.00012942 |
| chr17.0039668625 | −0.6680851 | 0.00012943 |
| chr15.0081868244 | 0.66127938 | 0.00012953 |
| chr18.0066099378 | 0.76537907 | 0.00012997 |
| chr2.0052234373 | 0.59860518 | 0.0001303 |
| chr3.0008596627 | −0.4921313 | 0.00013199 |
| chr2.0014703212 | 0.56959956 | 0.0001321 |
| chr17.0003094227 | 0.51203222 | 0.00013253 |
| chr2.0197922806 | 0.55879648 | 0.00013289 |
| chr8.0034939531 | −0.8905808 | 0.00013323 |
| chr15.0022699380 | 1.11253072 | 0.00013334 |
| chr18.0028620847 | −0.8646754 | 0.00013395 |
| chr5.0132461033 | 0.64861771 | 0.00013448 |
| chr21.0046714358 | 0.4767568 | 0.0001347 |
| chr8.0097611414 | 0.54007996 | 0.00013476 |
| chr12.0111419833 | 0.72480151 | 0.00013557 |
| chr7.0131266774 | 0.52367964 | 0.00013672 |
| chr7.0046699333 | −0.57638 | 0.00013709 |
| chr2.0194087232 | 0.50806509 | 0.00013718 |
| chr6.0029641397 | −0.5596024 | 0.00013725 |
| chr15.0067383802 | −1.0220357 | 0.00013733 |
| chr9.0021979074 | −0.7314048 | 0.00013772 |
| chr7.0115948932 | 0.58082301 | 0.00013783 |
| chr15.0061359896 | 0.65097307 | 0.00013788 |
| chr3.0126062999 | −0.6017227 | 0.00013819 |
| chr11.0121059993 | 0.580827 | 0.00013819 |
| chr20.0000425678 | 0.4691693 | 0.00013846 |
| chr11.0115355388 | 0.56837567 | 0.00013852 |
| chr8.0037774386 | 0.60769172 | 0.00013911 |
| chr11.0019533311 | 0.65982074 | 0.00013969 |
| chr17.0002466352 | −0.5785097 | 0.00013979 |
| chr2.0095569158 | −0.5407132 | 0.00013986 |
| chr6.0075908403 | 0.64532498 | 0.00013991 |
| chr12.0066778222 | 0.68545661 | 0.00013996 |
| chr1.0183338941 | 0.27739549 | 0.00014024 |
| chr22.0037580271 | 0.51095735 | 0.00014056 |
| chr19.0056653987 | −0.6469762 | 0.00014057 |
| chr7.0029678648 | 0.57150229 | 0.00014074 |
| chr16.0055358884 | 1.27864251 | 0.00014099 |
| chr12.0114474417 | 0.66877851 | 0.00014115 |
| chr22.0023487027 | 0.77170662 | 0.00014126 |
| chr7.0119590709 | 0.41770923 | 0.00014148 |
| chr18.0064583273 | 0.74292032 | 0.00014198 |
| chr6.0138169749 | 0.56500756 | 0.00014205 |
| chr5.0133973278 | 0.54263779 | 0.00014244 |
| chr7.0004022328 | 0.53209705 | 0.00014259 |
| chr6.0044470127 | 0.70808418 | 0.00014268 |
| chr6.0112713200 | −0.4989216 | 0.00014293 |
| chr13.0112741875 | 0.73839234 | 0.00014306 |
| chr2.0074834852 | 0.43415387 | 0.00014352 |
| chr14.0086214299 | 0.63414978 | 0.00014357 |
| chr11.0069462011 | 0.50654813 | 0.00014395 |
| chr17.0041958160 | 0.59957907 | 0.00014461 |
| chr12.0080931744 | −0.6735792 | 0.00014476 |
| chr6.0157104914 | −0.6934027 | 0.00014486 |
| chr17.0037322231 | 0.45892477 | 0.00014493 |
| chrX.0018708000 | 0.42536814 | 0.00014551 |
| chr7.0029290225 | −0.7236172 | 0.00014595 |
| chr2.0121111699 | 0.51164371 | 0.00014596 |
| chr5.0158049548 | 0.50019252 | 0.00014613 |
| chr7.0142204584 | −0.4901525 | 0.0001463 |
| chr6.0067353131 | 0.62572572 | 0.00014719 |
| chr12.0080067144 | 0.67537517 | 0.00014762 |
| chr11.0073310453 | −0.9153121 | 0.0001478 |
| chr15.0077997691 | −0.5531458 | 0.00014784 |
| chr9.0090239907 | 0.55339041 | 0.00014841 |
| chr6.0002624309 | 0.55622293 | 0.00014842 |
| chr11.0065683397 | −0.5965533 | 0.00014847 |
| chr6.0070174243 | −0.6834289 | 0.00014899 |
| chr18.0059698875 | 0.67816505 | 0.00014933 |
| chr6.0021674454 | 0.59634186 | 0.0001496 |
| chr12.0122669300 | −0.9043944 | 0.00014974 |
| chr16.0081180855 | 0.55324359 | 0.00014978 |
| chr16.0006468255 | −0.7961549 | 0.00015055 |
| chr5.0006298466 | 0.59822059 | 0.00015082 |
| chr3.0177546014 | 0.69258312 | 0.00015086 |
| chr11.0047671296 | 0.54152725 | 0.00015087 |
| chr7.0064744254 | 0.61895348 | 0.00015177 |
| chr3.0115665168 | 0.65074196 | 0.00015215 |
| chr5.0059190048 | 0.95640023 | 0.00015232 |
| chr12.0095796283 | 0.5769386 | 0.00015306 |
| chr16.0055774628 | 0.6543349 | 0.00015342 |
| chr12.0051348365 | −0.4908943 | 0.0001535 |
| chr17.0019685950 | 0.60619744 | 0.00015407 |
| chr19.0030035155 | −0.6470044 | 0.00015421 |
| chr6.0033168503 | 0.72630682 | 0.0001544 |
| chr9.0129549161 | 0.59414994 | 0.00015543 |
| chr5.0032629973 | 0.60987866 | 0.0001555 |
| chr2.0068907984 | 0.64968464 | 0.00015566 |
| chr19.0058325397 | −0.6517402 | 0.0001562 |
| chr13.0040615593 | −0.4772434 | 0.00015626 |
| chr5.0161866888 | 0.73236164 | 0.00015672 |
| chr12.0068825805 | 0.63700973 | 0.00015704 |
| chr2.0102641100 | 0.60467166 | 0.0001571 |
| chr2.0014079711 | 0.65639074 | 0.00015764 |
| chr6.0041217606 | 0.76470019 | 0.00015797 |
| chr3.0131320510 | 0.7077745 | 0.00015869 |
| chr3.0179547392 | 0.57633484 | 0.00015893 |
| chr6.0115266016 | 0.58897275 | 0.00015954 |
| chr10.0034835167 | 0.57424062 | 0.00016028 |
| chr11.0100788141 | 0.68655548 | 0.00016042 |
| chr2.0133140530 | 0.61465758 | 0.00016047 |
| chrX.0005226303 | 0.39640373 | 0.00016102 |
| chr3.0061863085 | −1.1664816 | 0.00016105 |
| chr14.0043162153 | 0.67497253 | 0.0001614 |
| chr18.0067902914 | 0.54659541 | 0.00016169 |
| chr3.0013111050 | −0.7981349 | 0.00016173 |
| chr9.0138113565 | −0.581437 | 0.00016175 |
| chr11.0024437985 | 0.59941097 | 0.00016214 |
| chr3.0098450309 | 0.39225312 | 0.00016222 |
| chr5.0081957209 | 0.59910397 | 0.00016235 |
| chr20.0017949564 | 1.35185953 | 0.00016252 |
| chr13.0095354282 | 1.22985023 | 0.00016265 |
| chr2.0171472668 | 0.507337 | 0.00016314 |
| chr14.0022056085 | 0.57301431 | 0.00016325 |
| chr11.0097792653 | 0.55137723 | 0.00016332 |
| chrX.0016879018 | −0.3911215 | 0.00016343 |
| chr6.0085228031 | 0.72524734 | 0.00016374 |
| chr6.0151937976 | 0.63036434 | 0.0001643 |
| chr4.0053883341 | −0.5797999 | 0.00016448 |
| chr9.0003194000 | −0.5700591 | 0.00016449 |
| chr9.0006024424 | −0.8906099 | 0.0001645 |
| chr6.0030634509 | −0.5620374 | 0.00016577 |
| chrX.0025064702 | −0.7051305 | 0.00016616 |
| chr13.0101367757 | 0.61040628 | 0.00016667 |
| chrX.0035572400 | −0.4808322 | 0.00016673 |
| chr18.0044920319 | 0.73951496 | 0.00016677 |
| chr10.0045131036 | 0.46990457 | 0.00016687 |
| chr18.0000824412 | 0.70156778 | 0.00016695 |
| chr2.0108539911 | 0.51520061 | 0.00016713 |
| chr12.0132994498 | 0.46675385 | 0.00016722 |
| chr14.0033043580 | 0.61943159 | 0.00016728 |
| chr3.0121485705 | 0.57496441 | 0.00016728 |
| chr2.0239956142 | 0.59862864 | 0.00016736 |
| chr21.0027889156 | 0.39768413 | 0.00016821 |
| chr13.0085237138 | −0.5646227 | 0.0001698 |
| chr7.0090189111 | 0.58708484 | 0.00016993 |
| chr4.0145589394 | 0.44412779 | 0.00016998 |
| chr16.0031423074 | −0.5706156 | 0.00017025 |
| chr3.0094403080 | 0.65430347 | 0.00017065 |
| chr19.0011553559 | −0.5624252 | 0.00017123 |
| chr20.0025057894 | 0.87030682 | 0.00017147 |
| chr7.0053787891 | 0.53006227 | 0.00017181 |
| chr20.0030405575 | 0.60422255 | 0.00017218 |
| chr11.0070456804 | −0.5647362 | 0.000173 |
| chr20.0039498397 | 0.49044292 | 0.00017324 |
| chr3.0178640990 | −0.5933867 | 0.00017425 |
| chr9.0122690954 | 0.55917209 | 0.00017457 |
| chr5.0070308421 | 0.64139621 | 0.00017512 |
| chr1.0016884648 | −0.3320293 | 0.00017578 |
| chr3.0110943669 | 0.65978581 | 0.00017593 |
| chr5.0004244902 | 0.76235004 | 0.00017661 |
| chr14.0093503539 | −0.6243695 | 0.00017694 |
| chr20.0061389865 | −0.7867361 | 0.00017733 |
| chr7.0140366526 | −0.478156 | 0.00017801 |
| chr12.0099759902 | 0.57317295 | 0.00017804 |
| chr2.0230616416 | 0.55038433 | 0.0001783 |
| chr5.0024572507 | 0.53208715 | 0.0001784 |
| chr5.0036023612 | 0.61009604 | 0.00017852 |
| chrX.0029658568 | 0.4634939 | 0.00017888 |
| chr15.0094421404 | 0.67335338 | 0.0001792 |
| chr5.0034075463 | 0.48555991 | 0.00017966 |
| chr14.0053459119 | −0.6265418 | 0.00018015 |
| chr5.0167404963 | 0.56670961 | 0.00018048 |
| chr14.0041241499 | 0.63797894 | 0.00018057 |
| chr16.0004013578 | 1.22715219 | 0.00018111 |
| chr8.0039641339 | −0.5322916 | 0.00018146 |
| chr11.0040924202 | 0.67646219 | 0.00018169 |
| chr19.0022574787 | −0.4360497 | 0.00018255 |
| chr10.0013137686 | 0.56924946 | 0.00018269 |
| chr19.0006661025 | −0.5948254 | 0.00018272 |
| chr12.0081367901 | 0.59007781 | 0.00018316 |
| chr3.0028557558 | 0.62561062 | 0.00018326 |
| chr1.0061482151 | −0.2700545 | 0.00018337 |
| chr22.0019155480 | −0.7454938 | 0.00018418 |
| chr17.0074440931 | −0.5675222 | 0.0001847 |
| chr4.0128064321 | 0.53385701 | 0.00018537 |
| chr20.0059782975 | 0.57510393 | 0.00018568 |
| chr10.0072053420 | 0.55895582 | 0.000186 |
| chr9.0011056308 | 0.6209761 | 0.00018616 |
| chr15.0031382498 | 0.65596291 | 0.00018645 |
| chr8.0048939769 | −0.4431561 | 0.00018714 |
| chr17.0048953585 | 0.66412045 | 0.00018822 |
| chr16.0054421513 | −0.5761907 | 0.00018837 |
| chr17.0067863192 | 0.44592861 | 0.00018866 |
| chr9.0090022041 | 0.60673564 | 0.00018884 |
| chr3.0100567671 | −0.5530397 | 0.00018898 |
| chr3.0120500103 | 0.69732751 | 0.00018905 |
| chr2.0127265940 | 0.41652887 | 0.00019007 |
| chr4.0056074366 | 0.44374799 | 0.00019014 |
| chr15.0099465151 | 0.49474931 | 0.0001911 |
| chr18.0033856062 | 0.71951488 | 0.00019122 |
| chr2.0044465132 | 0.58157873 | 0.00019152 |
| chr9.0023220200 | −0.6991691 | 0.00019157 |
| chr3.0048574848 | 0.59436895 | 0.00019184 |
| chr18.0058752488 | 0.66187068 | 0.00019225 |
| chr10.0123992661 | 0.56481638 | 0.00019352 |
| chr2.0198613561 | 0.46198016 | 0.00019384 |
| chr8.0036421076 | 0.6717421 | 0.0001939 |
| chr13.0027457057 | 0.48030573 | 0.00019407 |
| chr22.0050688843 | −0.5854609 | 0.00019501 |
| chr7.0133932344 | 0.61828397 | 0.00019538 |
| chr2.0097423379 | 0.52673604 | 0.0001977 |
| chr2.0049176835 | −0.6684947 | 0.0001978 |
| chr5.0165352291 | −0.6281675 | 0.00019828 |
| chr16.0066290265 | 0.59846957 | 0.0001984 |
| chr15.0088957178 | 0.73053305 | 0.00019875 |
| chr2.0096727954 | 0.56323859 | 0.00019875 |
| chr12.0126066470 | 0.62172689 | 0.00019904 |
| chrX.0074280722 | −0.3399894 | 0.00019931 |
| chr6.0037912821 | 0.55042421 | 0.00019961 |
| chr5.0173915088 | 0.47950895 | 0.00019993 |
| chrX.0108392212 | 0.42724731 | 0.00019994 |
| chr3.0126200659 | 0.52272201 | 0.00020051 |
| chr13.0103047839 | 0.67806796 | 0.00020078 |
| chr19.0041104393 | −1.1530403 | 0.000201 |
| chr6.0009417142 | 0.61422993 | 0.0002016 |
| chrX.0040916103 | −0.3560982 | 0.00020165 |
| chr20.0004925421 | 0.75695767 | 0.00020189 |
| chr16.0018058778 | 0.52449001 | 0.00020235 |
| chr19.0030985709 | −0.5410119 | 0.00020238 |
| chr9.0043545142 | 0.59129482 | 0.00020247 |
| chr7.0047873608 | 0.563916 | 0.0002025 |
| chr9.0140373789 | 0.47265915 | 0.00020261 |
| chr16.0018066408 | −0.5966407 | 0.00020335 |
| chr6.0036082115 | 0.60899649 | 0.00020345 |
| chr14.0086040376 | 0.61556417 | 0.0002037 |
| chr20.0001375701 | −1.1915254 | 0.00020411 |
| chr6.0047312268 | 0.56575294 | 0.0002043 |
| chr19.0043111029 | −0.9650931 | 0.0002055 |
| chr18.0024101773 | −1.1234776 | 0.00020572 |
| chr2.0237200683 | 0.50578563 | 0.00020583 |
| chr7.0090870611 | 0.57310893 | 0.00020617 |
| chr2.0027973063 | 0.53148012 | 0.00020641 |
| chr7.0003978134 | −0.3257868 | 0.00020655 |
| chr12.0121542540 | −0.496468 | 0.00020695 |
| chr18.0040644942 | 0.63316759 | 0.00020762 |
| chr3.0196689326 | 0.58679416 | 0.00020822 |
| chr5.0007540955 | −0.7307535 | 0.00020875 |
| chr5.0010790189 | 0.6154188 | 0.00020949 |
| chrX.0025037945 | 0.58203454 | 0.00020965 |
| chr12.0011949206 | 0.72891399 | 0.00021006 |
| chr20.0036829301 | 0.51890604 | 0.00021038 |
| chr5.0139759321 | 0.47316663 | 0.00021044 |
| chr13.0030715790 | −0.6149365 | 0.00021046 |
| chr10.0125263803 | 0.53444811 | 0.00021078 |
| chr10.0118568670 | 0.55204039 | 0.0002112 |
| chr22.0021240107 | 0.82410164 | 0.00021126 |
| chr15.0041474566 | 0.52762412 | 0.00021141 |
| chr11.0119176896 | −0.5705926 | 0.00021148 |
| chr22.0037326211 | 0.60020709 | 0.00021148 |
| chr6.0055533119 | 0.67213428 | 0.00021208 |
| chr17.0068449338 | 0.52555591 | 0.00021248 |
| chr6.0027604585 | 0.55128626 | 0.00021336 |
| chr7.0120298951 | 0.57567464 | 0.00021359 |
| chr7.0025529818 | 0.55193882 | 0.00021383 |
| chr7.0000394875 | 0.41695454 | 0.00021395 |
| chr3.0153128830 | −0.6152852 | 0.00021421 |
| chr15.0040886106 | 2.58724071 | 0.00021445 |
| chr6.0114343352 | 0.58135111 | 0.00021449 |
| chr4.0036305326 | −0.6590556 | 0.00021555 |
| chr17.0080982156 | −0.5362566 | 0.00021601 |
| chr11.0016626851 | 1.0726951 | 0.00021714 |
| chr13.0034909982 | 0.48392215 | 0.00021735 |
| chr18.0068627287 | 0.64784639 | 0.00021739 |
| chr12.0038828107 | 0.65516979 | 0.00021782 |
| chr6.0134954034 | −0.8511063 | 0.00021789 |
| chr11.0074008137 | −0.8292176 | 0.00021858 |
| chr5.0010855455 | 0.52900211 | 0.00021906 |
| chr13.0083219887 | −0.6996308 | 0.00021946 |
| chr5.0050914746 | 0.64959081 | 0.00021996 |
| chr2.0086815266 | −0.4119607 | 0.0002202 |
| chr4.0110793399 | 0.50955689 | 0.00022044 |
| chr15.0022432684 | 0.58874983 | 0.00022078 |
| chr18.0054461835 | −0.4588131 | 0.00022155 |
| chr1.0017959453 | −0.2692006 | 0.00022226 |
| chrX.0100852797 | −0.3773613 | 0.00022261 |
| chr1.0205504480 | 0.27076622 | 0.00022335 |
| chr17.0059652469 | −0.7746947 | 0.00022349 |
| chr13.0095358801 | −0.9388267 | 0.00022389 |
| chr20.0042889263 | 0.53694202 | 0.00022416 |
| chr6.0071596636 | 0.63977168 | 0.00022447 |
| chr7.0070974791 | −0.4885714 | 0.00022454 |
| chr16.0024200887 | 0.57255346 | 0.00022459 |
| chr2.0085813555 | −0.494364 | 0.00022479 |
| chr15.0084561206 | 0.54782685 | 0.00022561 |
| chr4.0124988349 | 0.49001159 | 0.00022636 |
| chr7.0034938658 | 0.55001794 | 0.00022639 |
| chr14.0104850057 | −0.5819994 | 0.00022641 |
| chr8.0142747656 | 0.65789324 | 0.00022678 |
| chr2.0229922587 | 0.56930728 | 0.00022755 |
| chr2.0045233435 | −0.8705792 | 0.00022826 |
| chr14.0040851604 | 0.55257115 | 0.00022855 |
| chr3.0063661912 | −0.6515679 | 0.0002295 |
| chr13.0112314434 | −0.5619942 | 0.00022963 |
| chr5.0139150800 | 0.69664672 | 0.00023008 |
| chr2.0019438569 | −0.4881268 | 0.00023035 |
| chr1.0041952802 | 0.33272278 | 0.00023045 |
| chr20.0045717508 | 0.59317294 | 0.0002305 |

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of determining breast cancer status of a subject, the method comprising:
determining a methylation state for each of a plurality of cytosine-guanine dinucleotide (CpG) sites in a sample obtained from the subject,
calculating a cancer presence differential methylation level and an invasiveness differential methylation level based on the methylation states of the plurality of CpG sites, and
comparing the cancer presence differential methylation level and the invasiveness differential methylation level to a predetermined cancer status reference level and a predetermined invasiveness reference level,
wherein when the cancer presence differential methylation level deviates from the predetermined cancer status reference level, the presence of breast cancer is indicated in the subject, and
when the invasiveness differential methylation level deviates from the predetermined invasiveness reference level, the presence of invasive breast cancer is indicated in the subject.

2. A method of detecting breast cancer in a subject, the method comprising:
determining a methylation state for each of a plurality of cytosine-guanine dinucleotide (CpG) sites in a sample obtained from the subject,
calculating a cancer status differential methylation level based on the methylation states of the plurality of CpG sites, and
comparing the cancer status reference differential methylation level to a predetermined reference level,
wherein when the cancer status differential methylation level deviates from the predetermined reference level, the presence of breast cancer is indicated in the subject.

3. A method of determining if breast cancer in a subject is invasive, or non-invasive, the method comprising:
determining a methylation state for each of a plurality of cytosine-guanine dinucleotide (CpG) sites in a sample obtained from the subject,
calculating an invasiveness differential methylation level based on the methylation states of the plurality of CpG sites, and
comparing the invasiveness differential methylation level to a predetermined reference level,
wherein when the differential methylation level deviates from the predetermined reference level, the breast cancer in the subject is invasive.

4. The method according to claim 1, wherein the plurality of CpG sites comprises at least one selected from the CpG sites listed in Table 3 or Table 15.

5. The method according to claim 3, wherein the plurality of CpG sites comprises at least five selected from the CpG sites listed in Table 21.

6. The method according to claim 1, wherein the plurality of CpG sites comprises at least ten selected from the CpG sites listed in Table 3 or Table 15.

7. The method according to claim 3, wherein the plurality of CpG sites comprises at least ten selected from the CpG sites listed in Table 21.

8. The method according to claim 1, wherein the plurality of CpG sites comprises at least m % selected from the top n most predictive CpG sites listed in Table 3 and/or Table 15, wherein:
m is selected from the group consisting of: 50, 60, 70, 80, 90, 95, and 99; and
n is selected from the group consisting of 25, 50, 100, 500 and 1,000.

9. The method according to claim 3, wherein the plurality of CpG sites comprises at least m % selected from the top n most predictive CpG sites listed in Table 21, wherein:
m is selected from the group consisting of: 50, 60, 70, 80, 90, 95, and 99; and
n is selected from the group consisting of 25, 50, 100, 500 and 1,000.

10. The method according to claim 1, further comprising:
providing treatment for breast cancer to the subject when breast cancer is indicated.

11. The method of claim 8 wherein treatment for breast cancer comprises the administration of medication, radiation or surgery.

12. The method according to claim 1, wherein calculating a differential methylation level comprises adding in a linear weighted summation values based on the methylation states of the plurality of CpG sites.

13. The method according to claim 1, wherein the sample is a blood sample.

14. The method according to claim 1, wherein the sample is tumor tissue.

15. The method according to claim 1, wherein the subject has or is suspected to have ductal cell in situ carcinoma.

16. The method according to claim 1, wherein the subject has or is suspected to have triple-negative breast cancer.

17. The method according to claim 1, wherein the subject has or is suspected to have hormone receptor positive (ER⁺PR⁺) breast cancer.

18. The method according to claim 1, wherein the subject has or is suspected to have HER2⁺ breast cancer.

19. The method according to claim 1, wherein the subject is being monitored for the local or systemic recurrence of breast cancer.

20. The method of claim 3, wherein the plurality of CpG sites comprises at least one selected from the CpG sites listed in Table 27.
